(19) United States
(12) Patent Application Publication
(10) Pub. No.: US 2008/0221070 A1
(43) Pub. Date: Sep. 11, 2008

(54) METHODS AND COMPOUNDS FOR THE TARGETED DELIVERY OF AGENTS TO BONE FOR INTERACTION THEREWITH

(76) Inventors: William M. Pierce, Louisville, KY (US); K. Grant Taylor, Louisville, KY (US); Leonard C. Waite, Corydon, IN (US)

Correspondence Address: STITES & HARBISON PLLC, 401 COMMERCE STREET, SUITE 800, NASHVILLE, TN, 37219, US

(21) Appl. No.: 12/043,904

(22) Filed: Mar. 6, 2008

Related U.S. Application Data
(60) Provisional application No. 60/893,366, filed on Mar. 6, 2007.

(51) Int. Cl. A61K 31/665; C07J 41/00; A61K 31/661; A61P 19/10; A61P 19/08; C07F 9/08

(52) U.S. Cl. 514/100; 552/506; 514/119; 549/220

(57) ABSTRACT

Bone targeted compounds and methods are provided. Compounds can include a Bone Targeting Portion (R_{T}), having an affinity for bone; a Bone Active Portion (R_{A}) for interacting with and affecting bone; a Linking Portion (R_{L}) connecting the Bone Targeting Portion and the Bone Active Portion. The Bone Active Portion is derived from an estrogenic agent. Compounds can also include a Blocking Group (R_{P}) that reduces or eliminates the estrogenic activity of the Bone Active Portion.

### RELATED APPLICATIONS

This application claims priority from U.S. Provisional Application Ser. No. 60/893,366 filed Mar. 6, 2007, the entire disclosure of which is incorporated herein by this reference.

### TECHNICAL FIELD

The presently-disclosed subject matter relates to treatment of bone disorders and conditions, and, more particularly, to the targeted delivery of prophylactic and therapeutic agents to bone.

### BACKGROUND

Bone is a dynamic tissue, consisting of cells in a protein matrix, upon which is superimposed a crystalline structure of various calcium salts. Because bone is the primary structural support system for the body of an animal, bone disorders can create substantial problems. Bone disorders include, for example, fractures, suboptimal mechanical competence, suboptimal bone blood perfusion, suboptimal bone healing ability, cancerous transformation (primary bone cancer and metastasis of cancer to bone), and infection.

Bone disorders can occur in a variety of manners. For example, bone disorders can result from excessive forces being exerted onto the bone, primary bone conditions, and secondary bone conditions associated with other conditions. Bone conditions include, for example, metabolic bone diseases (MBDs). MBDs are conditions characterized by weakening of bones, which weakening is associated with suboptimal mechanical competence and an increased likelihood of fracturing. Osteoporosis is an example of a MBD. Osteoporosis is characterized by bone degeneration caused by a relative excess of bone resorption. Clinical osteoporosis is found in approximately 25% of postmenopausal women, and subclinical osteoporosis, which is responsible for untold numbers of bone fractures, is far more widespread. Other examples of MBDs include, but are not limited to: Paget's disease, which is characterized by an abnormal growth of bone such that the bone is larger and weaker than normal bone; and osteogenesis imperfecta, which is characterized by bones that are abnormally brittle.

In addition to serving as a rigid support for the body of an animal, bone is an organ that responds to various agents. To the extent that bone has the ability to interact with and respond to certain agents, disorders associated with bone conditions can be prevented, diagnosed, or treated using appropriate agents having the ability to interact with and affect a desired response in bone. For example, with regard to osteoporosis, there are certain agents, which are thought to interact with bone and are currently available for the treatment or prevention of the condition. Such agents include: bisphosphonates (e.g., alendronate, risedronate); calcitonin; selective estrogen receptor modulators (SERMs) (e.g., raloxifene); selective androgen receptor modulators (SARMs); growth factors; cytokines; agents used for estrogen or hormone replacement therapy (ET/HRT); and parathyroid hormone (PTH) (e.g., teriparatide).

There are a variety of disadvantages associated with treatment using these known agents. For example, although PTH has some anabolic activity, biphosphonates, calcitonin, SERMs, and ET/RHT are primarily anti-catabolic, operating to limit bone resorption. In this regard, the anti-catabolic compounds only treat osteoporosis in so much as they attempt to keep bone density from further decreasing. There are also various side effects associated with such agents; for example, bisphosphonate treatment is associated with gastrointestinal and esophageal erosion, and has been implicated in osteonecrosis of the jaw; SERM treatment has been associated with deep vein thrombosis and hot flashes; ET/HRT has been implicated in increased risk of breast cancer and cardiovascular disease; and PTH therapy has been suggested to potentially increase the risk of osteosarcoma (osteogenic sarcoma), a type of cancer that develops in bone and is characterized by formation of a bone matrix having decreased strength relative to normal non-malignant bone matrix, and which can metastasize to other bones and other organs. See e.g., Bilezikian J P (2006) N Engl J Med 355:2278-2281; Cranney A, Adachi J D (2005) Drug Saf 28:721-730; Marshall J K (2002) Expert Opin Drug Saf 1:71-78; Rossouw J E, et al., Writing Group for the Women's Health Initiative Investigators (2002) Risks and benefits of estrogen plus progestin in healthy postmenopausal women: principal results From the Women's Health Initiative randomized controlled trial. JAMA 288:321-333; Vahle J L, et al. (2002) Toxicol Pathol 30:312-321, which are incorporated herein by this reference. There are also various drawbacks associated with the delivery of such known agents to an animal, for example, bisphosphonates demonstrate poor oral bioavailability, calcitonin is not orally deliverable, and PTH must be injected. Additionally, some known agents have a limited capacity to affect bone because they lack a specific affinity for bone. That is to say that when some of the known agents are delivered to an animal, they are not specifically directed to the bone. In this regard, when some of the known agents are delivered to an animal, they are delivered to non-specific locations in the body of the animal, such that they fail to interact with the bone or require a large dose to affect a response in bone. Also in this regard, when such agents are delivered to an animal, they can be directed to undesirable locations in the body of the animal, resulting in undesirable side effects.

Accordingly, there remains a need in the art for compounds, systems, and methods for treating bone disorders and conditions that satisfactorily address some or all of the above-identified disadvantages.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The compounds of the presently-disclosed subject matter can be represented by the following formula:

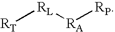

R_{T} represents a Bone Targeting Portion. R_{L} represents a linking portion that separates and connects the Bone Targeting Portion and a Bone Active Portion. R_{A} represents a Bone Active Portion derived from an estrogenic agent. R_{P} represents a Blocking Group that reduces or eliminates the estrogenic activity of the Bone Active Portion. The Bone Targeting Portion (R_{T}) can be represented by the following formula:

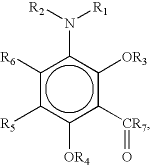

and can be connected at R₁, R₂, R₄, or R₇, to the Linking Portion (R_{L}). R₁ can be hydrogen, lower alkyl, alkyl, aryl lower alkyl, or aryl, when R_{T} is not connected at R₁ to R_{L};R₂ can be hydrogen, lower alkyl, alkyl, aryl lower alkyl, or aryl, when R_{T} is not connected at R₂ to R_{L}; R₃ can be hydrogen, lower alkyl, alkyl, aryl lower alkyl, aryl, or carbonyl-containing; R₄ can be hydrogen, lower alkyl, alkyl, aryl lower alkyl, aryl, or carbonyl-containing, when R_{T} is not connected at R₄ to R_{L}; R₅ and R₆ can be independently hydrogen, lower alkyl, or alkyl, or R₅ and R₆, taken together with the carbon atoms to which they are bonded, form a ring containing about 6 to about 14 carbon atoms and up to a total of about 18 carbon atoms, which formed ring can be monocyclic, bicyclic, or tricyclic, wherein the ring can have substituents, including heteroatoms; R₇ can be hydroxy, lower alkoxy, or NR₈, R₉, when R_{T} is not connected at R₇ to R_{L}; and R₈ and R₉ can be independently hydrogen, or lower alkyl.

In some embodiments, R₃ is hydrogen. In some embodiments, R₅ and R₆ are hydrogen. In some embodiments, R₇ is NR₈R₉. In some embodiments, R₈ and R₉ are both hydrogen. In some embodiments, R₁ is hydrogen or aryl, when R_{T} is not connected at R₁ to R_{L}; R₂ is hydrogen or aryl, when R_{T} is not connected at R₂ to R_{L}; R₄ is lower alkyl, or hydrogen, when R_{T} is not connected at R₄ to R_{L}; R₃, R₅, and R₆ are each hydrogen; and R₇ is NH₂, when R_{T} is not connected at R₇.

In some embodiments, the compound can be represented by the formula

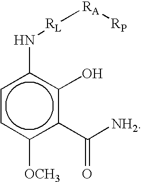

In some embodiments, the compound can be represented by the formula

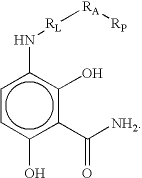

In some embodiments, the compound can be represented by the formula

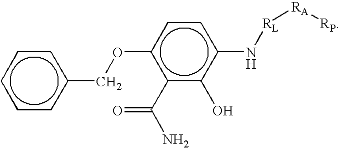

In some embodiments, the compound can be represented by the formula

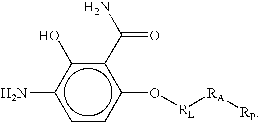

In some embodiments, R₅ and R₆ taken together with the carbon atoms to which they are attached form a ring containing between 6 and 14 ring carbon atoms, the ring being monocyclic, bicyclic, or tricyclic.

In some embodiments, R₁ is hydrogen, lower alkyl, or aryl, when R_{T} is not connected at R₁ to R_{L}; R₂ is hydrogen or aryl, when R_{T} is not connected at R₂ to R_{L}; R₃ is hydrogen, or lower alkyl; R₄ is hydrogen, when R_{T} is not connected at R₄; R₅, and R₆ are each hydrogen; and R₇ is NH₂, when R_{T} is not connected at R₇. In some embodiments, R₁ is hydrogen, and R₃ is hydrogen.

In some embodiments, the Blocking Group is derived from: esters and ethers formed by condensation of lower alkyl, alkyl, or aryl; and sulfates, phosphates, phosphonates, bisphosphonates, substituted bisphosphonates, and salts, esters, or ethers thereof. In some embodiments, the Blocking Group is derived from phosphates, phosphonates, bisphosphonates, substituted bisphosphonates, and salts, esters, or ethers thereof. In some embodiments, the blocking group is derived from phosphate, etidronate, clodronate, tiludronate, pamidronate, alendronate, neridronate, olpadronate, ibandronate, risedronate, zoledronate, minodronate, incadronate, or EB-1053.

In some embodiments, the Bone Active Portion is derived from an estrogenic agent selected from: estradiol; estrone; estriol; an estrogen precursor; an estrogen analogue; an estrogen metabolite; tibolone; 2-methoxyestradiol; genistein; resveratrol; daidzein; glycitein; formononetin; biochanin A; diethylstilbestrol; enterodiol; enterolactone; hexestrol; xenoestrogens; phytoestrogens; mycoestrogens; coumestrol; a coumestan; isoflavonoids; ipriflavone; secoisolariciresinol diglycoside; and lignan phytoestrogens.

In some embodiments, the compound can be represented by the formula

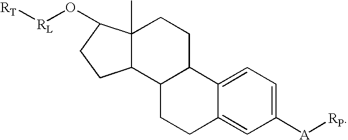

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by a formula selected from

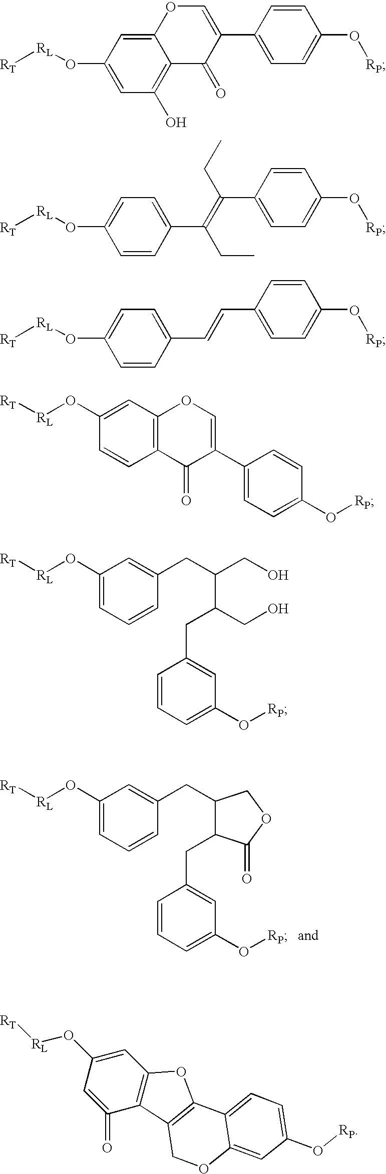

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by the formula

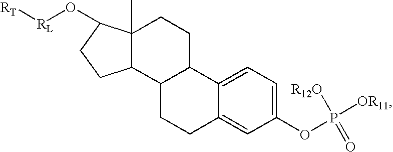

wherein R₁₁ and R₁₂ are independently hydrogen, lower alkyl, alkyl, or aryl. In some embodiments, R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

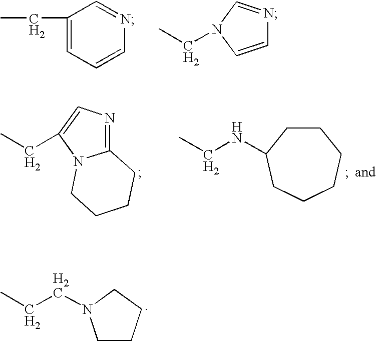

In some embodiments, the compound can be represented by the formula

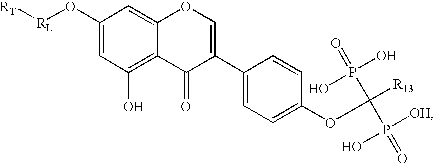

wherein R₁₃ is hydrogen, lower alkyl, alkyl, or aryl. In some embodiments, R₁₃ is selected from: —H; —CH₃; —Cl;

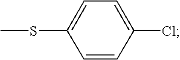

(CH₂)₂NH₂; —(CH₂)₃NH₂; —(CH₂)₄NH₂; —(CH₂)₅NH₂; —(CH₂)₂N(CH₃)₂;

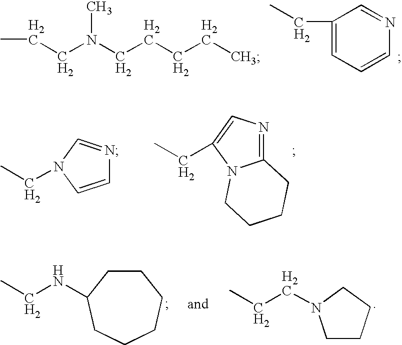

In some embodiments, the compound can be represented by the formula

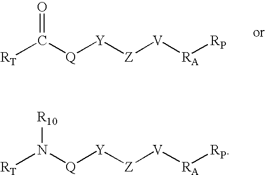

R₁₀ is independently hydrogen or lower alkyl. Q is a straight or branched alkylene group, containing 1 to about 10 carbon atoms on a main chain, and up to a total of about 20 carbon atoms. Y is —C—,

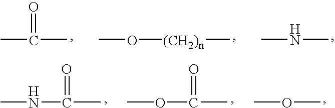

or a chemical bond; Z is

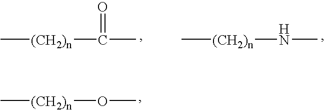

or a chemical bond; and V is

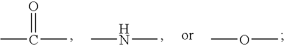

provided Y-E-V is

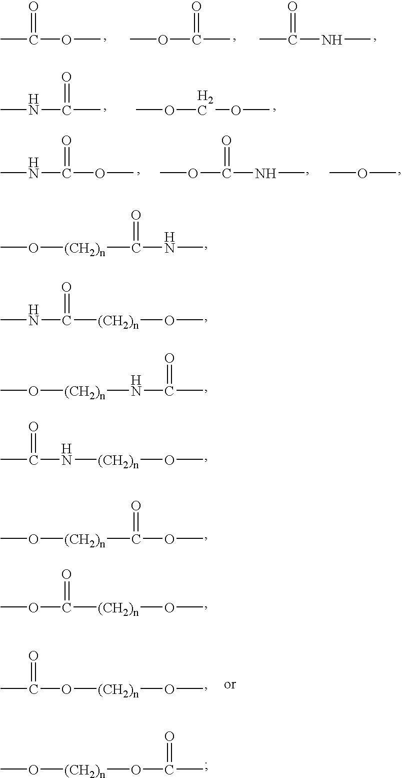

and n is an integer from 0 to 6. In some embodiments, the compound can be represented by the formula

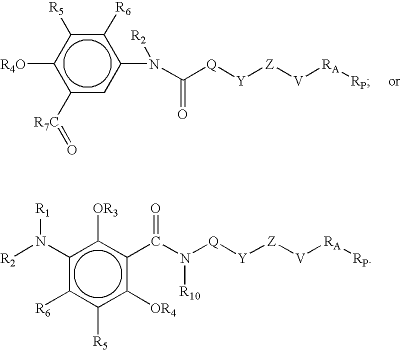

In some embodiments, the compound can be represented by the formula

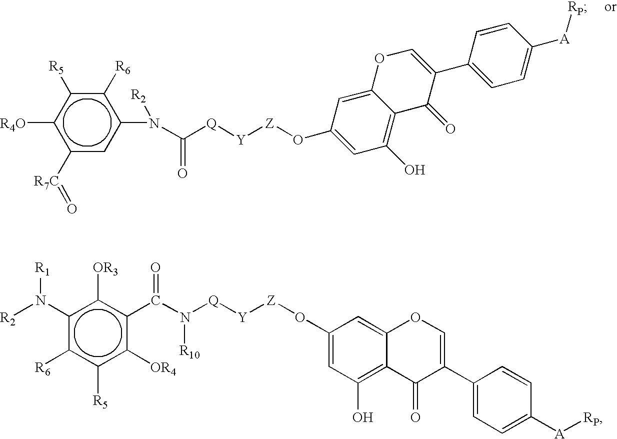

wherein A is a heteroatom.

In some embodiments, the compound can be represented by the formula

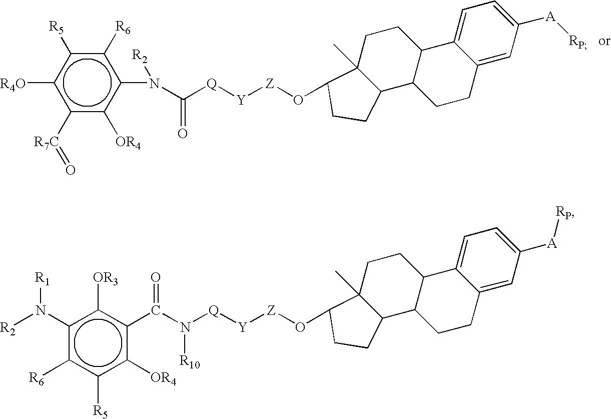

wherein A is a heteroatom.

In some embodiments, the compound can be represented by the formula

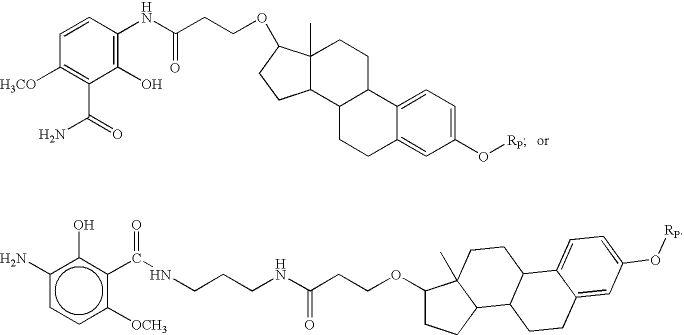

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by the formula

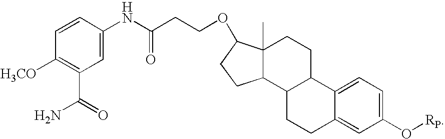

In some embodiments, the compound can be represented by the formula

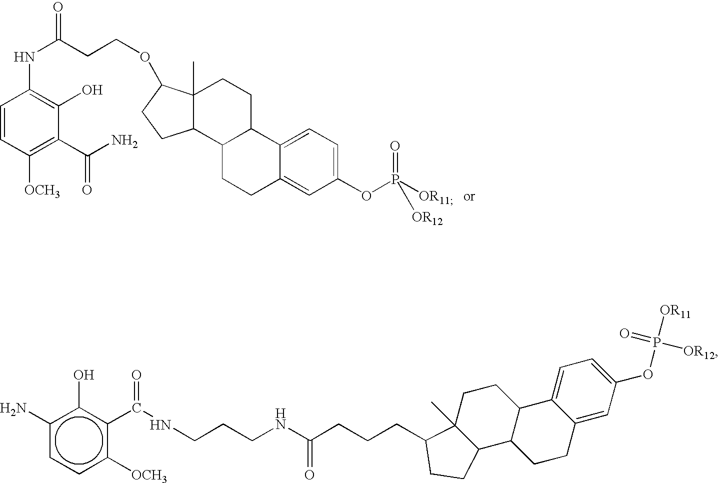

wherein R₁₁ and R₁₂ are independently hydrogen, lower alkyl, alkyl, or aryl. In some embodiments, R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

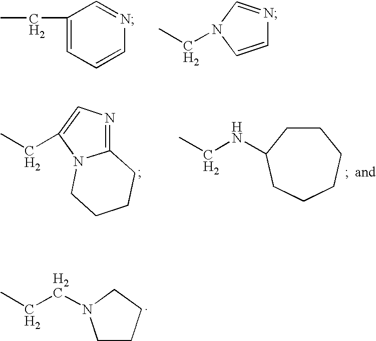

In some embodiments, the compound can be represented by the formula

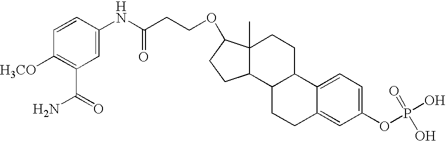

In some embodiments, the compound can be represented by the formula

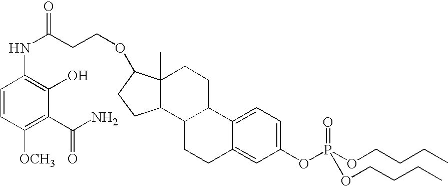

In some embodiments, the compound can be represented by the formula

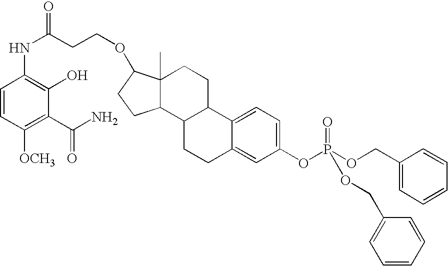

In some embodiments, the compound can be represented by the formula

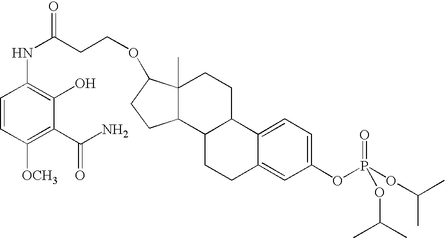

In some embodiments, the compound can be represented by the formula

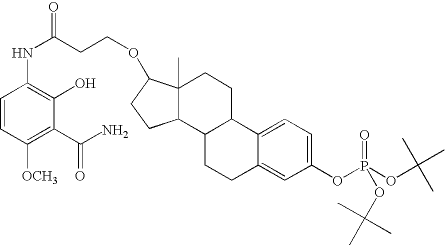

In some embodiments, the compound can be represented by the formula

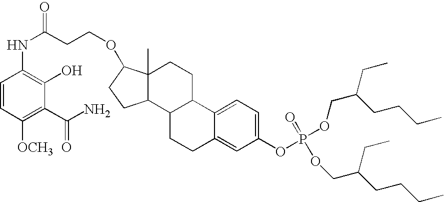

In some embodiments, the compound can be represented by the formula

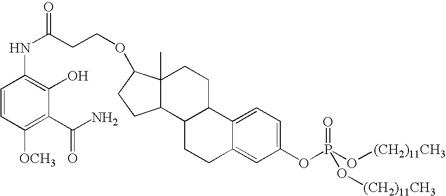

In some embodiments, the compound can be represented by the formula

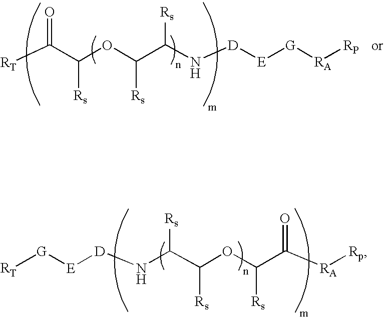

wherein m is 1-3, n is 1-4, and when m>1, each n is independently 1-4; each R_{S} is independently hydrogen, lower alkyl, or lower alkyl with heteroatoms; D and G are independently covalent bond, carbonyl, epoxy, or anhydride; and E is covalent bond, (CT2)ᵣ, where T is hydrogen, hydroxy, or lower alkyl, and where r is 0-8, or (C)ᵣ, where r is 2-8, and where the carbons are unsaturated or partially saturated with hydrogen.

In some embodiments, the compound can be represented by the formula

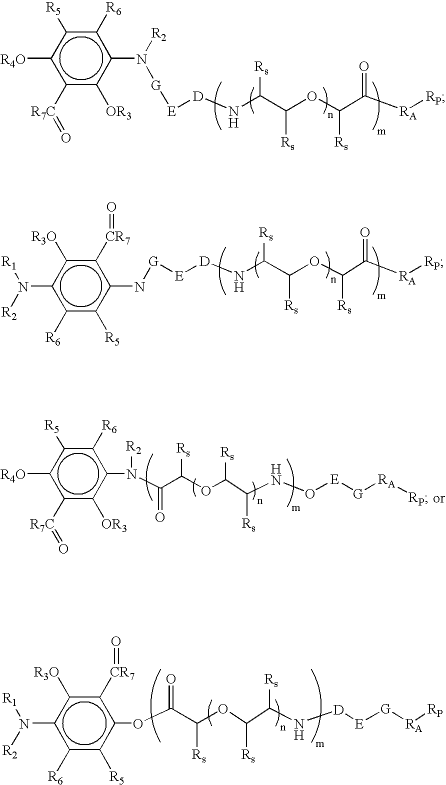

In some embodiments, the compound can be represented by the formula

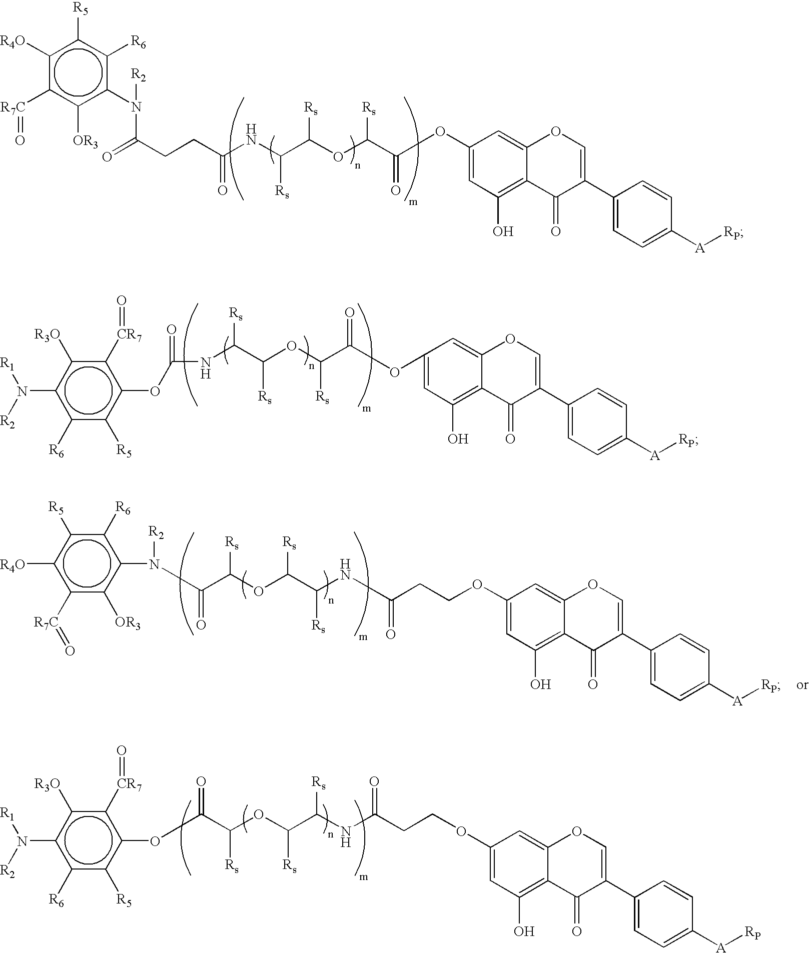

In some embodiments, the compound can be represented by the formula

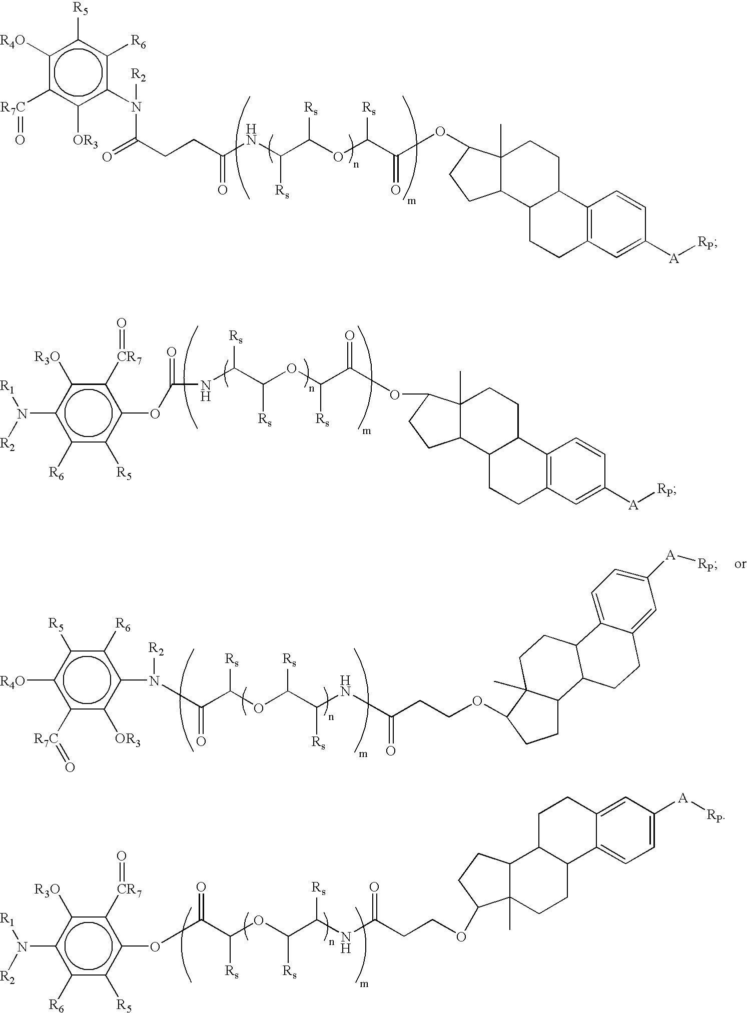

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by the formula

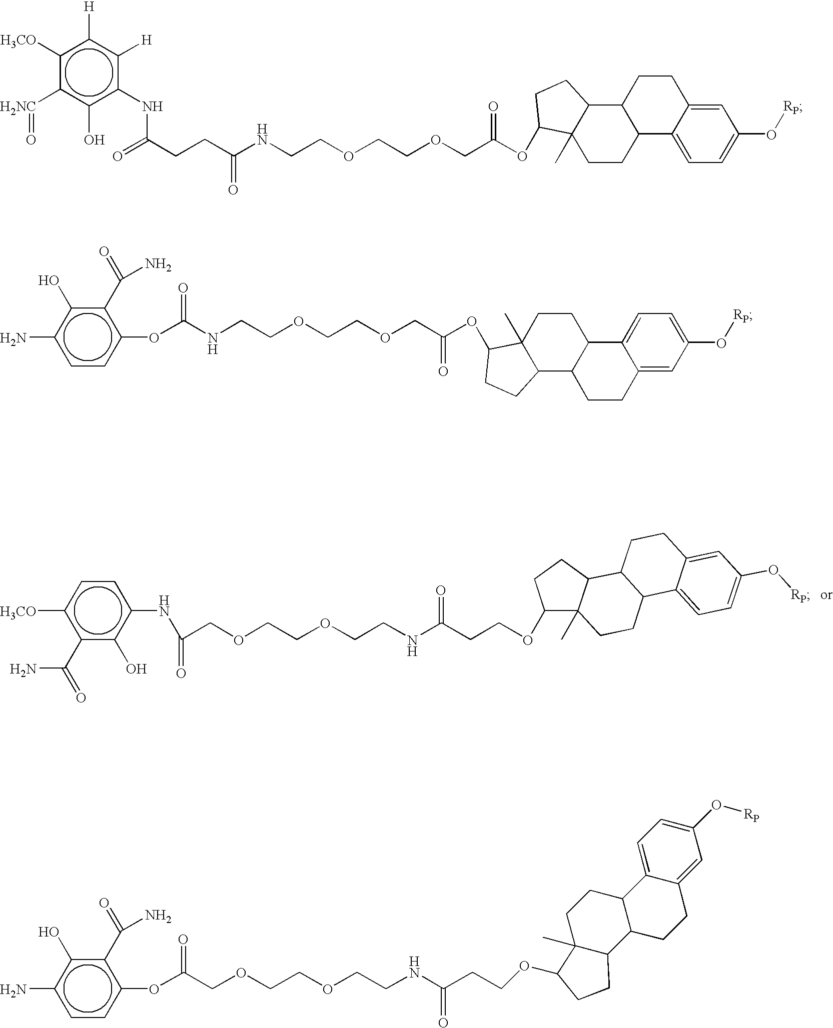

In some embodiments, the compound can be represented by the formula

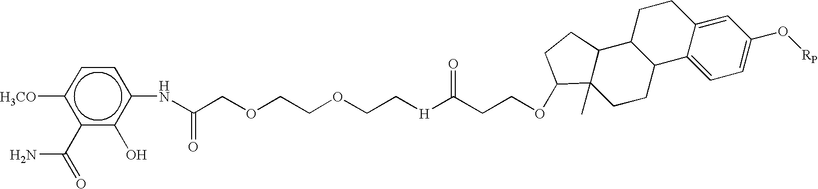

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by the formula

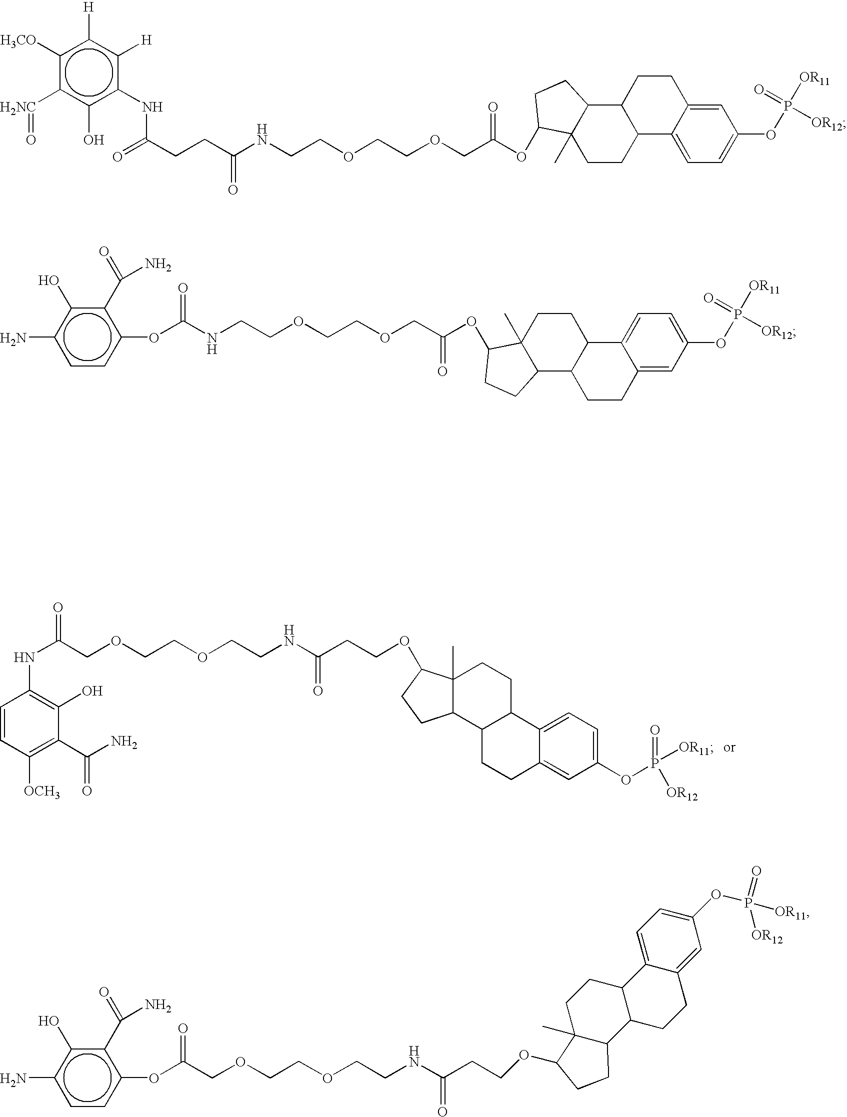

wherein R₁₁ and R₁₂ are independently hydrogen, lower alkyl, alkyl, or aryl. In some embodiments, R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

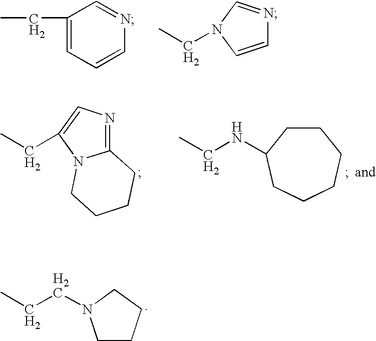

In some embodiments, the compound can be represented by the formula

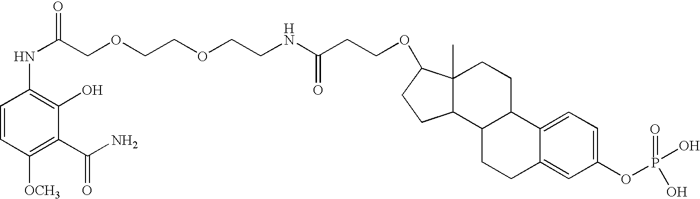

In some embodiments, the compound can be represented by the formula

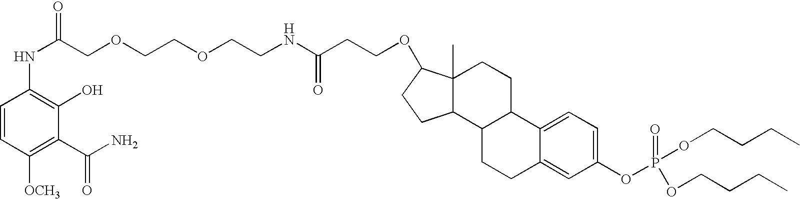

In some embodiments, the compound can be represented by the formula

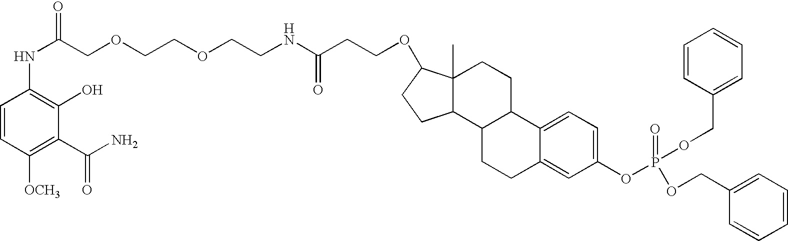

In some embodiments, the compound can be represented by the formula

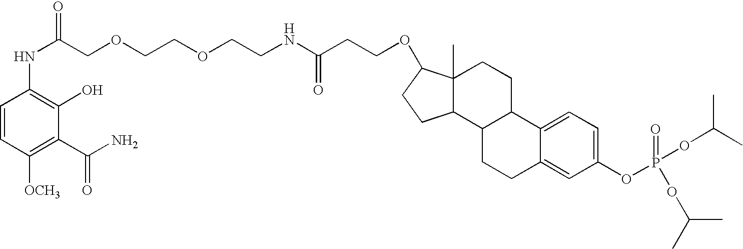

In some embodiments, the compound can be represented by the formula

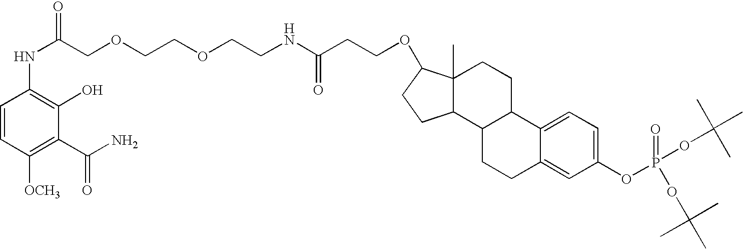

In some embodiments, the compound can be represented by the formula

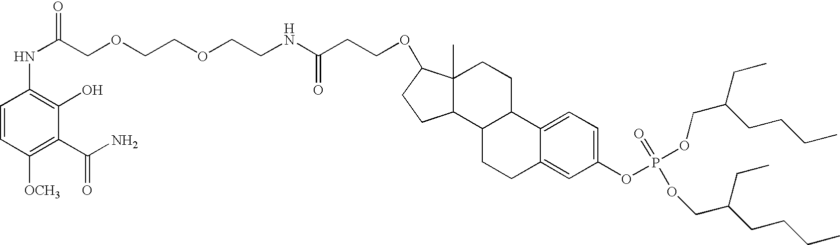

In some embodiments, the compound can be represented by the formula

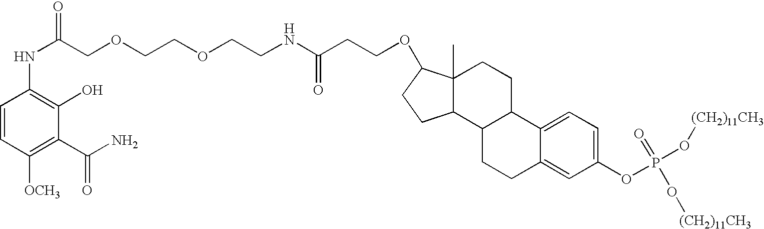

In some embodiments, the compound can be represented by the formula

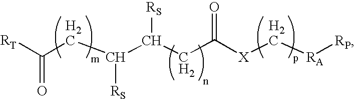

where m is 0-3, n is 0-3, and p is 0-4; each R_{S} is independently hydrogen or hydroxy; and X is O, NH, S, or covalent bond.

In some embodiments, the compound can be represented by the formula

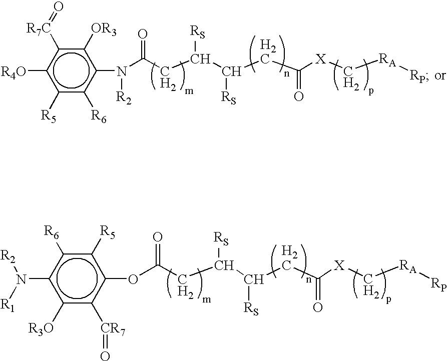

In some embodiments, the compound can be represented by the formula

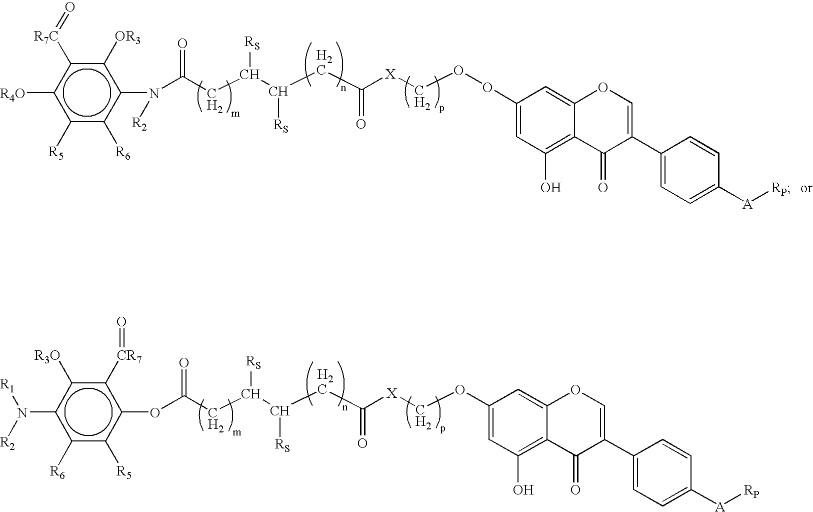

In some embodiments, the compound can be represented by the formula

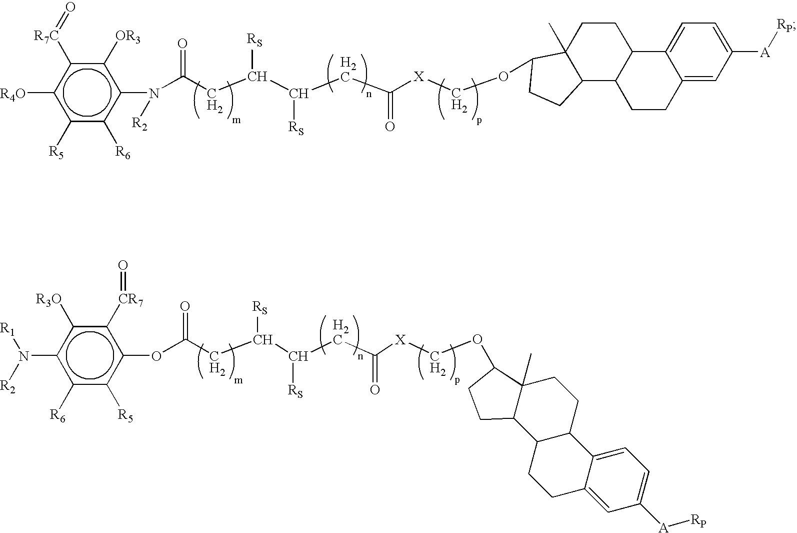

In some embodiments, R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

In some embodiments, the compound can be represented by the formula

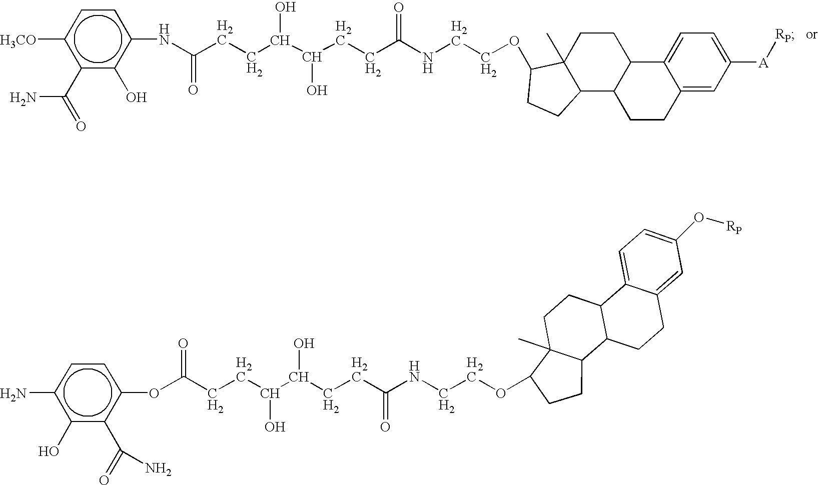

In some embodiments, the compound can be represented by the formula

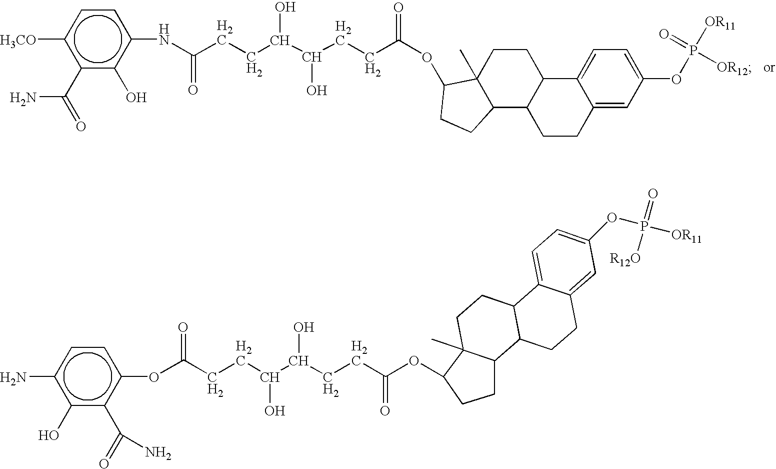

wherein R₁₁ and R₁₂ are independently hydrogen, lower alkyl, alkyl, or aryl. In some embodiments, R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

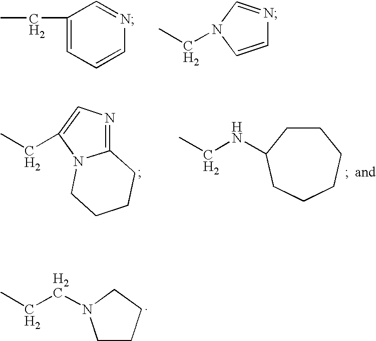

The presently-disclosed subject matter includes a method for treating a bone condition in an subject, comprising administering to the subject an effective amount of a compound as described herein. In some embodiments, the bone condition is a metabolic bone disorder. In some embodiments, the metabolic bone disorder is osteoporosis. In some embodiments, the bone condition is a fracture. In some embodiments, administering the compound has an anti-catabolic effect and/or an anabolic effect on the bone of the subject. In some embodiments, administering the compound has an anabolic effect on the bone of the subject.

### BRIEF DESCRIPTION OF THE DRAWINGS

FIG. 1 is a line graph depicting body weight as a function of time for animals administered 17-ethinyl estradiol, alendronate, parathyroid hormone, the compound of Formula 158 (BTA-2), or the compound of Formula 159 (BTA-3).

FIG. 2 is a bar graph depicting the uterine mass of animals administered 17-ethinyl estradiol, alendronate, parathyroid hormone, the compound of Formula 158 (BTA-2), or the compound of Formula 159 (BTA-3).

FIG. 3 is a bar graph depicting the ratio of uterine mass to body weight of animals administered 17-ethinyl estradiol, alendronate, parathyroid hormone, the compound of Formula 158 (BTA-2), or the compound of Formula 159 (BTA-3).

FIG. 4 is a bar graph depicting the whole bone density of animals administered 17-ethinyl estradiol, alendronate, parathyroid hormone, the compound of Formula 158 (BTA-2), or the compound of Formula 159 (BTA-3).

FIG. 5 is a bar graph depicting the regional bone density of the proximal left femur of animals administered 17-ethinyl estradiol, alendronate, parathyroid hormone, the compound of Formula 158 (BTA-2), or the compound of Formula 159 (BTA-3).

FIG. 6 is a bar graph depicting the regional bone density of the distal left femur of animals administered 17-ethinyl estradiol, alendronate, parathyroid hormone, the compound of Formula 158 (BTA-2), or the compound of Formula 159 (BTA-3).

FIG. 7 is a line graph depicting body weight as a function of time for animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 8 is a bar graph depicting the uterine mass of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 9 is a bar graph depicting the ratio of uterine mass to body weight of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 10 is a bar graph depicting the whole bone density of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 11 is a bar graph depicting the regional bone density of the proximal left femur of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 12 is a bar graph depicting the regional bone density of the distal left femur of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 13 is a bar graph depicting the regional bone density of the left femoral diaphysis of animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 14 is a bar graph illustrating trabecular volume fraction data for animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 15 includes three-dimensional images of bone that were constructed using data collected by a customized micro-CT system, for animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 16 is a bar graph depicting bone resorption or osteoclast-mediated breakdown of collagen type I in bone by measuring the C-telopeptide fragment of collagen type I (CTX-I) in animals administered 17-ethinyl estradiol, alendronate, parathyroid hormone, the compound of Formula 66 (BTE2-D2-3-O-DBtP), or the compound of Formula 67 (BTE2-D2-3-O-DBnP).

FIG. 17 is a bar graph depicting serum osteocalcin levels for animals administered 17-ethinyl estradiol, alendronate, parathyroid hormone, the compound of Formula 66 (BTE2-D2-3-O-DBtP), or the compound of Formula 67 (BTE2-D2-3-O-DBnP).

### DESCRIPTION OF EXEMPLARY EMBODIMENTS

The details of one or more embodiments of the presently-disclosed subject matter are set forth in this document. Modifications to embodiments described herein, and other embodiments, will be evident to those of ordinary skill in the art after a study of the information provided in this document. The information provided herein, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used in the present specification, the following words and phrases are generally intended to have the meanings as set forth below, except to the extent that the context in which they are used indicates otherwise.

“Lower alkyl,” refers to alkyl groups with the general formula CₙH₂ₙ₊₁, where n=1 to about 6. In some embodiments, n=1 to about 3. The groups can be straight-chained or branched. Examples include methyl, ethyl, propyl, isopropyl, n-butyl, sec-butyl, t-butyl, isobutyl, n-pentyl, isopentyl, neopentyl, n-hexyl, and the like.

“Lower alkyl with heteroatoms,” refers to groups with the general formula CₙXₘHᵣ, where X is a heteroatom, and n+m=2 to about 6. In some embodiments, n+m=2 to about 3. The heteroatom can be selected from: nitrogen, oxygen, sulfur, phosphorus, boron, chlorine, bromine, iodine, and other heteroatoms. In some embodiments, the heteroatom is selected from: nitrogen, oxygen, and sulfur. r=a positive whole number (integer) that is appropriate in light of n, X, and m, as will be understood by one of ordinary skill in the art. For example, if n=2, X is nitrogen, and m=1, then r=6, such that the group is C₂H₆. The groups can be straight-chained or branched.

“Alkyl,” refers to alkyl groups with the general formula CₙH₂ₙ₊₁, where n=about 6 to about 18. The groups can be straight-chained or branched. In some embodiments the group can be a cyclo-alkyl.

“Alkyl with heteroatoms,” when used alone or in combination with other groups, refers to groups with the general formula CₙXₘHᵣ, where X is a heteroatom, and n+m=about 6 to about 18. The heteroatom can be selected from: nitrogen, oxygen, sulfur, phosphorus, boron, chlorine, bromine, iodine, and other heteroatoms. In some embodiments, the heteroatom is selected from: nitrogen, oxygen, and sulfur. r=a positive whole number (integer) that is appropriate in light of n, X, and m, as will be understood by one of ordinary skill in the art. For example, if n=5, X is nitrogen, and m=2, then r=13, such that the group is C₅N₂H₁₃. The groups can be straight-chained or branched.

“Carbonyl-containing,” refers to a group containing a carbonyl, for example, an aldehyde, a ketone, an ester, an amide, a carboxylic acid, or an acyl group. The groups can include 1 to about 6 carbon atoms, and at least one oxygen atom.

“Aryl,” refers to an aromatic group containing ring carbon atoms and having about 5 to about 14 ring carbon atoms and up to a total of about 18 ring or pendant carbon atoms. Examples include phenyl, α-naphthyl, β-naphthyl, tolyl, xylyl, and the like.

“Aryl lower alkyl” refers to an aryl group bonded to a bridging lower alkyl group, as defined herein. Examples include benzyl, phenethyl, naphthylethyl, and the like.

Each of the aforementioned groups could be substituted or unsubstituted. For example, “alkyl” can include substituted alkyl, substituted with hydroxyl, heteroatoms, or lower alkyl groups.

The presently-disclosed subject matter includes compounds, or pharmaceutically-acceptable compositions thereof, having an affinity for bone, or “bone targeted compounds.” The presently-disclosed subject matter includes bone targeted compounds and methods useful for treating conditions of interest, e.g., conditions affecting bone. The presently-disclosed subject matter further includes methods for delivering an agent of interest to bone.

The bone targeting compounds of the presently-disclosed subject matter can be described as including multiple units. In some embodiments, the compounds include the following units: a Bone Targeting Portion, having an affinity for bone; a Bone Active Portion, capable of interacting with bone; a Blocking Group modifying the Bone Active Portion; and a Linking Portion that separates and connects the Bone Targeting Portion and the Bone Active Portion.

The compounds of the presently-disclosed subject matter can be represented by the following formula:

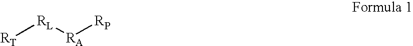

where, R_{T} represents the Bone Targeting Portion, R_{L} represents the linking portion, R_{A} represents the Bone Active Portion, and R_{P} represents the Blocking Group modifying the Bone Active Portion. In some embodiments, the compounds can be provided as a salt, solvate, ester, or ether, e.g., a pharmaceutically-acceptable salt, solvate, ester, or ether.

The Bone Targeting Portion of the compounds has an affinity for bone, allowing the compounds to be directed to bone of a subject. The Linking Portion of the compounds connect and separate the Bone Targeting Portion and the Bone Active Portion, which, without wishing to be bound by theory, is believed to limit steric interference of the Bone Active Portion when interacting with bone. The Bone Active Portion is derived from an estrogenic agent and can interact with and affect a response in the bone. Estrogenic agents can have estrogenic activity associated with undesirable side effects. In this regard, the Bone Active Portion can be modified by the Blocking Group. When the Bone Active Portion is modified by the Blocking Group, the estrogenic activity of the Bone Active Portion can be reduced or eliminated. When the compound has been directed to the bone, the Blocking Group can be cleaved from the compound, restoring the activity of the Bone Active Portion such that it can interact with and affect a reaction in the bone, the intended target of the activity.

The Bone Targeting Portion allows the Bone Active Portion to be specifically directed to the bone of the subject, thereby reducing delivery to non-specific locations and increasing the bioavailability of the Bone Active Portion at the target site. The targeted delivery allows for Bone Active Portions of compounds to be derived from estrogenic agents that fail to interact with bone when administered as free estrogenic agents, despite beneficial activities that may be associated with the free estrogenic agents. The targeted delivery can allow for the use of smaller doses of compounds including Bone Active Portions, as compared to treatment using free estrogenic agents lacking a specific affinity for bone.

Additionally, by specifically directing the Bone Active Portion of the compounds to bone, there is a reduced risk of directing the Bone Active Portion to undesirable locations in the body of the subject, resulting in undesirable side effects. For example, agents having estrogenic activity can interact with bone to treat or prevent metabolic bone diseases and/or fractures; however, the binding of such estrogenic agents to extraosseous estrogen receptors, e.g., in breast or ovarian tissue, has been associated with the development of cancer.

The Blocking Group can serve to further reduce the risk of undesirable side effects associated with administration of compounds having estrogenic activity. By modifying the Bone Active Portion with a Blocking Group, the estrogenic activity of the Bone Active Portion is reduced or eliminated, prior to its delivery to the bone. For example, by blocking the estrogenic activity of the Bone Active Portion prior to its delivery to the bone, binding of the Bone Active Portion to estrogen receptors (ER) of breast or ovarian tissue can be reduced or substantially eliminated. Without wishing to be bound by theory or mechanism, in some embodiments, Blocking Groups can be used that are thought to have an affinity for bone, which affinity is independent from the affinity provided by the Bone Targeting Portion. In this regard, in some embodiments, the Blocking Group can contribute to the targeted delivery of the compounds to the bone, and the associated benefits of such targeted delivery.

### Bone Targeting Portion

The Bone Targeting Portion (R_{T}) of the compound has an affinity for the extracellular inorganic matrix of bone. The Bone Targeting Portion can be represented by the following formula:

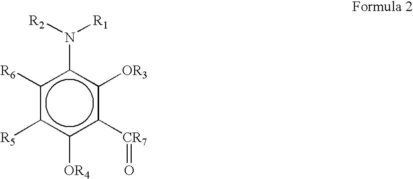

wherein

R₁ is hydrogen, lower alkyl, alkyl, aryl lower alkyl, or aryl;

R₂ is hydrogen, lower alkyl, alkyl, aryl lower alkyl, or aryl;

R₃ is hydrogen, lower alkyl, alkyl, aryl lower alkyl, aryl, or carbonyl-containing;

R₄ is hydrogen, lower alkyl, alkyl, aryl lower alkyl, aryl, or carbonyl-containing;

R₅ and R₆ are independently hydrogen, lower alkyl, or alkyl, or R₅ and R₆, taken together with the carbon atoms to which they are bonded, form a ring containing about 6 to about 14 ring carbon atoms and up to a total of about 18 carbon atoms, which formed ring can be monocyclic, bicyclic, or tricyclic, wherein the ring can optionally have substituents, including heteroatoms;

R₇ is hydroxy, lower alkoxy, or NR₈R₉ and

R₈ and R₉ are independently hydrogen, or lower alkyl.

An exemplary Bone Targeting Portion of the presently-disclosed subject matter can be represented by the following formula:

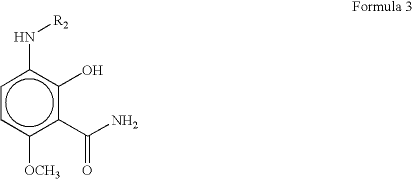

where R₁, R₃, R₅, and R₆ are each hydrogen; R₄ is methyl; and R₇ is amino.

Another exemplary Bone Targeting Portion of the presently-disclosed subject matter can be represented by the following formula:

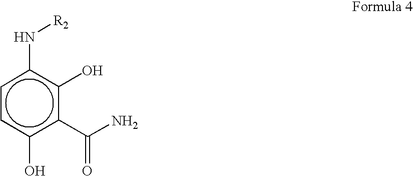

where R₁, R₃, R₄, R₅, and R₆ are each hydrogen; and R₇ is amino.

Another exemplary Bone Targeting Portion of the presently-disclosed subject matter can be represented by the following formula:

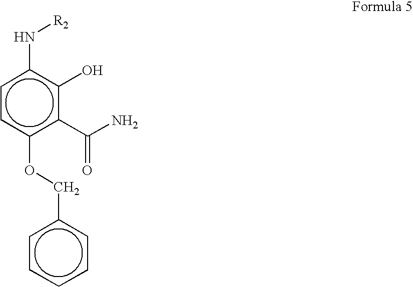

where R₁, R₃, R₅, and R₆ are each hydrogen; R₄ is benzyl; and R₇ is amino.

Another exemplary Bone Targeting Portion of the presently-disclosed subject matter can be represented by the following formula:

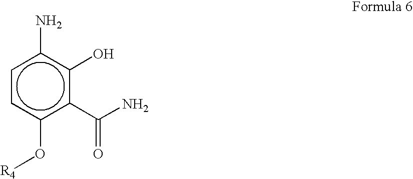

where R₁, R₂, R₃, R₅, and R₆ are each hydrogen; and R₇ is amino.

Another exemplary Bone Targeting Portion of the presently-disclosed subject matter can be represented by the following formula:

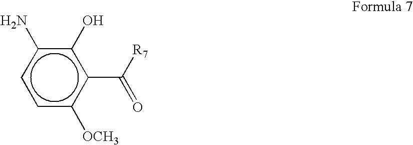

where R₁, R₂, R₃, R₅, and R₆ are each hydrogen; and R₄ is CH₃.

The linking portion is attached to the Bone Targeting Portion in the place of R₁, R₂, R₄, or R₇. For example, when the linking portion is attached to the Bone Targeting Portion in the place of R₁, the compound has the following formula:

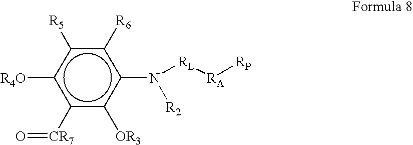

As another non-limiting example, when the linking portion is attached to the Bone Targeting Portion in the place of R₄, the compound has the following formula:

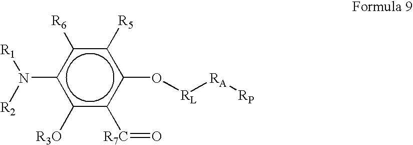

As another non-limiting example, when the linking portion is attached to the Bone Targeting Portion in the place of R₇, the compound has the following formula:

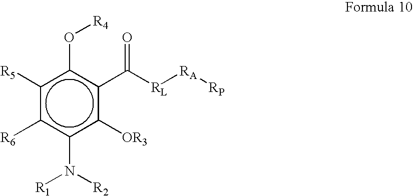

### Bone Active Portion (R_{A})

The Bone Active Portion of the compounds interacts with and affects bone. The Bone Active Portion can be derived from an estrogenic agent, that can be selected for its efficacy in treating a condition of interest, e.g., a metabolic bone diseases; a fracture. Estrogenic agents can be ER agonists, which bind to ERs and initiate a cellular response associated with an estrogen/ER binding complex.

Exemplary Bone Active Portions of the compounds of the presently-disclosed subject matter can be derived from estrogenic agents, including steroidal and non-steroidal estrogenic agents, including estrogens, estrogen precursors, plant-derived estrogens, and estrogen analogues and metabolites. Examples of estrogenic agents from which the Bone Active Portion of the compounds of the presently-disclosed subject matter can be derived include, but are not limited to, those set forth in Table A.

TABLE A
Examples of Estrogenic Agents from which the
Bone Active Portion can be Derived
Bone Active Agent (BAA)

| steroidal estrogenic agents, | |
|---|---|
| including but not limited | non-steroidal estrogenic agents, including but |
| to the following: | not limited to the following: |
| estradiol | genistein |
| estrone | resveratrol |
| estriol | daidzein |
| estrogen precursors | glycitein |
| estrogen analogues and | formononetin |
| metabolites | biochanin A |
| tibolone | diethylstilbestrol |
| 2-Methoxyestradiol (2-ME) | enterodiol |
| | enterolactone |
| | hexestrol |
| | xenoestrogens |
| | phytoestrogens & mycoestrogens |
| | coumestans (e.g., coumestrol) |
| | isoflavonoids |
| | ipriflavone |
| | lignan phytoestrogens (including but not |
| | limited to: secoisolariciresinol |
| | diglycoside) |

A Bone Active Portion that is derived from an estrogenic agent can be modified relative to the estrogenic agent as is necessary to be connected to the remainder of the compound, while maintaining some or all of the activity associated with the estrogenic agent, or while obtaining enhanced activity relative to the estrogenic agent. For example, a Bone Active Portion derived from an estrogenic agent after being linked to the compound can have the structure of the estrogenic agent, less a leaving group (e.g., less a hydrogen, less a hydroxyl, less a covalent bond, or less another leaving group) or including a connecting group, as will be apparent to one of ordinary skill in the art.

The Bone Active Portion can be additionally modified relative to the estrogenic agent in order to increase the bioavailability of the compound, and to reduce the risk of certain undesirable side effects. In this regard, the Bone Active Portion can be modified to include a Blocking Group that can reduce or eliminate the estrogenic activity of the Bone Active Portion, until the compound reaches the target bone. When the compound reaches the target bone, the blocking group can be cleaved, such that the Bone Active Portion is allowed to interact with the target bone. By blocking the estrogenic activity of the Bone Active Portion prior to its delivery to the target bone, undesirable extraosseous side effects can be reduced or eliminated.

Without wishing to be bound by theory or mechanism, once embodiments of the compound are delivered to bone for interaction therewith, the Bone Active Portion could be cleaved from the compound, becoming a free Bone Active Agent capable of interacting with adjacent bone. Alternatively, once embodiments of the compound are delivered to bone, the Bone Active Portion, as part of the bone-targeted compound, could interact with bone.

In some embodiments, the Bone Active Portion can be modified to include a Blocking Group at a location on the Bone Active Portion that is associated with estrogenic activity. For example, estrogenic agents from which the Bone Active Portion is derived (e.g., estrogenic agents set forth in Table A) can have a phenolic ring that is associated with estrogenic activity. Without wishing to be bound by theory or mechanism, the phenolic ring can effect binding of the agent to estrogen receptors, and it is thought that modifying the phenolic ring with a Blocking Group can reduce or eliminate the estrogenic activity of the agent.

For example, in some embodiments, the Bone Active Portion is derived from a steroid, wherein the phenolic ring is a steroidal A-ring of the Bone Active Portion. When the A-ring is modified to include a Blocking Group, in some embodiments, it can be modified at the 3-position. For example, in some embodiments, the Bone Active Portion is derived from estradiol and is modified at the 3-position of the A-ring, as represented by the following formula:

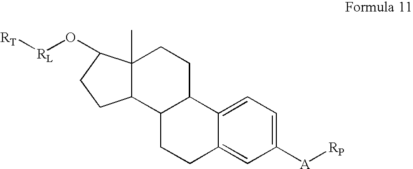

where A can be oxygen, or another heteroatom, for example, nitrogen, or sulfur; and where R_{P} is a Blocking Group. As shown in Formula II, the Bone Active Portion is estradiol less a hydrogen, allowing the Bone Active Portion to be connected to the remainder of the compound. In some embodiments, when the Bone Active Portion of the compound is derived from estradiol, it is derived from the 17-β-enantiomer of estradiol. Without wishing to be bound by theory or mechanism, it is believed that the 17-β-enantiomer of estradiol is the active isomer. Formula II also includes a blocking group (R_{P}) at the 3-position of the A-ring, reducing or eliminating estrogenic activity of the compound.

For another example, in some embodiments, the Bone Active Portion is derived from a non-steroidal estrogenic agent including a phenolic ring appropriate for modification by the Blocking Group. For example, in some embodiments, the Bone Active Portion is derived from genistein, as represented by the following formula:

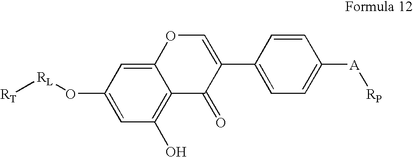

where A can be oxygen, or another heteroatom, for example, nitrogen, or sulfur; and where R_{P} is a Blocking Group. As shown in Formula 12, the Bone Active Portion is genistein less a hydrogen, allowing the Bone Active Portion to be connected to the compound, and less a second hydrogen, where the Blocking Group is connected to the compound. Formula 12 also includes a blocking group (R_{P}) on the phenolic ring, reducing or eliminating estrogenic activity of the compound. It is noted that in some embodiments, the Bone Active Portion can be derived from an agent having a modified phenolic ring. For example, biochanin A includes a phenolic ring, modified by a methyl (CH₃). In some embodiments, the Bone Active Portion can be derived from biochanin A, as represented by the following formula:

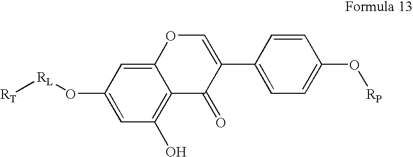

As shown in Formula 13, the Bone Active Portion is biochanin A less a hydrogen, allowing the Bone Active Portion to be connected to the compound, and less a methyl (CH₃), where the Blocking Group is connected to the compound.

For another example, in some embodiments, the Bone Active Portion is derived from diethylstilbestrol, as represented by the following formula:

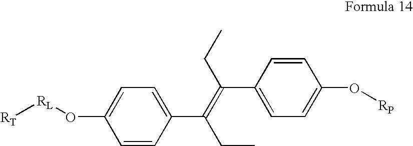

For another example, in some embodiments, the Bone Active Portion is derived from resveratrol, as represented by the following formula:

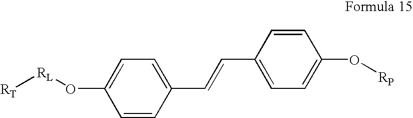

For another example, in some embodiments, the Bone Active Portion is derived from daidzein, as represented by the following formula:

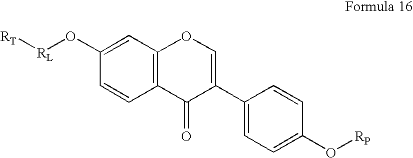

For another example, in some embodiments, the Bone Active Portion is derived from enterodiol, as represented by the following formula:

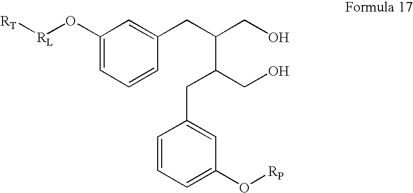

For another example, in some embodiments, the Bone Active Portion is derived from enterolactone, as represented by the following formula:

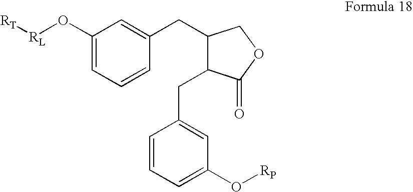

For another example, in some embodiments, the Bone Active Portion is derived from coumestrol, as represented by the following formula:

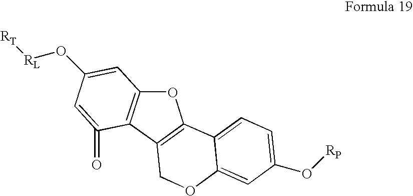

When the compound is delivered to the target bone, the Blocking Group (R_{P}) can be cleaved such that the Bone Active Portion has one or more activities generally associated with the free estrogenic agent (e.g., estradiol, genistein, biochanin A, diethylstilbestrol, resveratrol, daidzein, enterodiol, enterolactone, or coumestrol, in the case of Formulas 11-19), and is allowed to interact with and affect bone. The Blocking Group can be cleaved, for example, by enzymes produced by bone that are capable of hydrolyzing the Blocking Group. For example, when the Blocking Group is derived from a phosphate, it can be cleaved by a bone phosphatase.

In some embodiments, the Blocking Group (R_{P}) can include: esters or ethers formed by condensation of lower alkyl, alkyl, or aryl; or sulfates, phosphates, phosphonates, bisphosphonates, substituted bisphosphonates, or esters or ethers thereof, or salts or solvates thereof. In some embodiments, the blocking group (R_{P}) can be derived from phosphorate, etidronate, clodronate, tiludronate, pamidronate, alendronate, neridronate, olpadronate, ibandronate, risedronate, zoledronate, minodronate, incadronate, or EB-1053. See Russell, “Bisphosphonates: from Bench to Bedside,” (2006) Annals New York Academy of Science, pp. 367-401, which is incorporated herein by this reference. A Blocking Group that is derived from a particular agent can be modified relative to that agent as is necessary to be connected to the remainder of the compound. For example, a Blocking Group derived from an agent after being linked to the compound can have the structure of the agent, less a leaving group or including a connecting group, as will be apparent to one of ordinary skill in the art.

In some embodiments, the Blocking Group can be derived from a phosphate, as represented by the following formula:

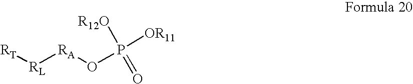

In some embodiments, the compounds can be provided wherein R₁₁ and R₁₂ can be independently selected from groups as set forth in Table B.

TABLE B

| | |
|---|---|
| —H | 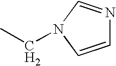 |
| —CH₃ | 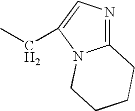 |
| —(CH₂)₂NH₂ | 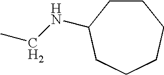 |
| —(CH₂)₃NH₂ | 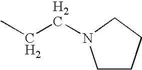 |
| —(CH₂)₄NH₂ | |
| —(CH₂)₅NH₂ | |
| —(CH₂)₂N(CH₃)₂ | |
| 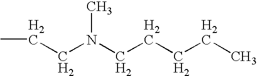 | |
| 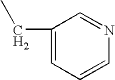 | |

In some embodiments, the compound can be represented by the following formula, where R_{P} is derived from di-n-butyl phosphate:

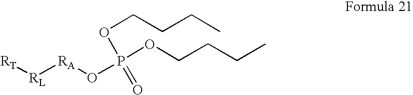

In some embodiments, the compound can be represented by the following formula, where R_{P} is derived from dibenzyl phosphate:

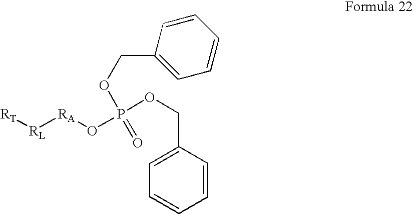

In some embodiments, the compound can be represented by the following formula, where R_{P} is derived from dibenzyl phosphate:

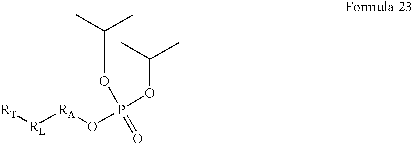

In some embodiments, the compound can be represented by the following formula, where R_{P} is derived from di-tert-butyl phosphate:

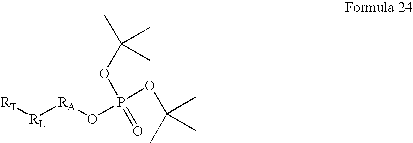

In some embodiments, the compound can be represented by the following formula, where R_{P} is derived from di-2-ethylhexyl phosphate:

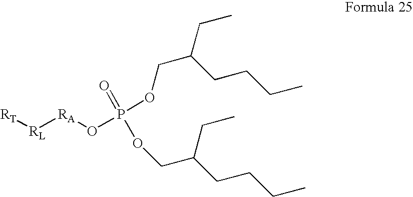

In some embodiments, the compound can be represented by the following formula, where R_{P} is derived from didodecyl phosphate:

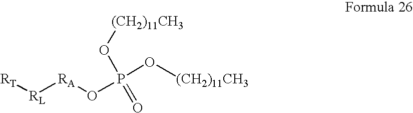

In some embodiments, the Blocking Group can be derived from a bisphosphonate, as represented by the following formula:

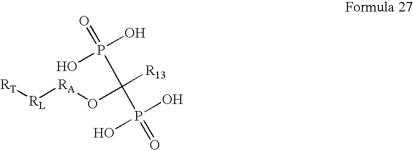

where R_{T} is the Bone Targeting Portion, R_{L} is the Linking Portion, and where R₁₃ can be hydrogen, lower alkyl, alkyl, or aryl. In some embodiments, R₁₃ can be a group as set forth in Table C.

TABLE C

| | |
|---|---|
| —H | 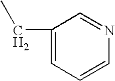 |
| —CH₃ | 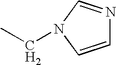 |
| —Cl | 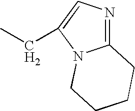 |
| 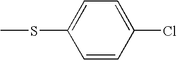 | 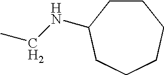 |
| —(CH₂)₂NH₂ | 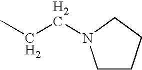 |
| —(CH₂)₃NH₂ | |
| —(CH₂)₄NH₂ | |
| —(CH₂)₅NH₂ | |
| —(CH₂)₂N(CH₃)₂ | |
| 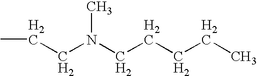 | |

In some embodiments, the Bone Active Portion is derived from estradiol and is modified with a Blocking Group at the 3-position of the A-ring, as represented by the following formula:

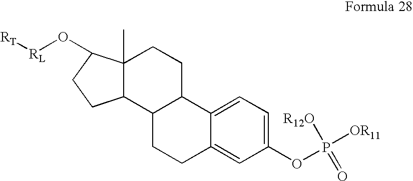

where R_{T} is the Bone Targeting Portion, R_{L} is the Linking Portion, and where R₁₁ and R₁₂ can be independently hydrogen, lower alkyl, alkyl, or aryl. When the compound is delivered to the target bone, the blocking group (R_{P}) can be cleaved, e.g., by a bone phosphatase, such that the Bone Active Portion has one or more activities generally associated with free estradiol, and is allowed to interact with and affect bone.

In some embodiments, the compound can be represented by the following formula, where R_{P} is derived from di-n-butyl phosphate:

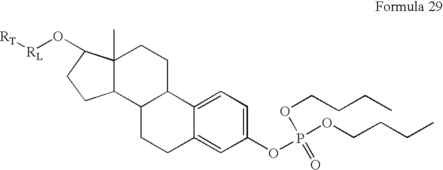

In some embodiments, the compound can be represented by the following formula, where R_{P} is derived from dibenzyl phosphate:

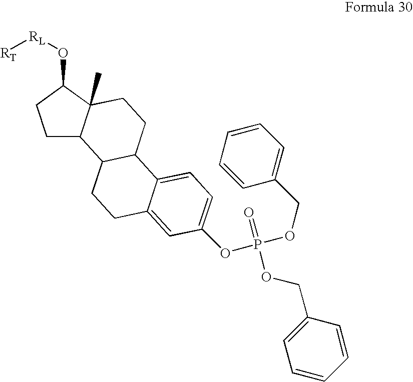

In some embodiments, the compound can be represented by the following formula, where R_{P} is derived from dibenzyl phosphate:

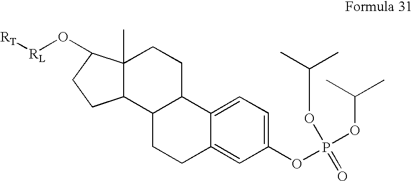

In some embodiments, the compound can be represented by the following formula, where R_{P} is derived from di-tert-butyl phosphate:

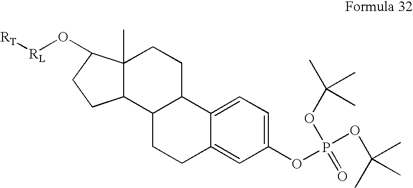

In some embodiments, the compound can be represented by the following formula, where R_{P} is derived from di-2-ethylhexyl phosphate:

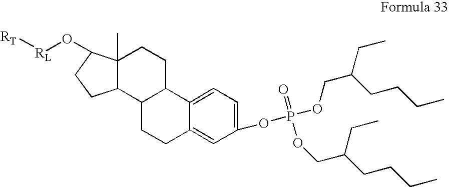

In some embodiments, the compound can be represented by the following formula, where R_{P} is derived from didodecyl phosphate:

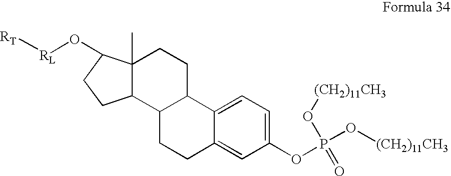

As another non-limiting example, when the Bone Active Portion is derived from estradiol and is modified with another exemplary Blocking Group at the 3-position of the A-ring, the compound can be represented by the following formula:

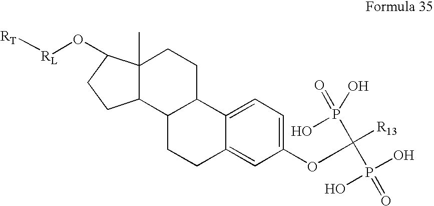

where R_{T} is the Bone Targeting Portion, R_{L} is the Linking Portion, and where R₁₃ can be hydrogen, lower alkyl, alkyl, or aryl. R₁₃ can be selected from the groups as set forth in Table C.

In some embodiments, the Bone Active Portion is derived from a non-steroidal estrogenic agent. In some embodiments, the Bone Active Portion is derived from genistein and is modified with a Blocking Group, as represented by the following formula:

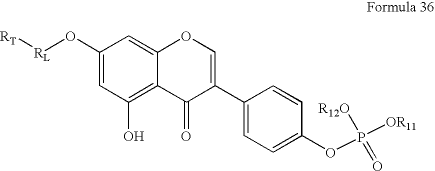

where R_{T} is the Bone Targeting Portion, R_{L} is the Linking Portion, and where R₁₁ and R₁₂ can be independently hydrogen, lower alkyl, alkyl, or aryl. When the compound is delivered to the target bone, the blocking group (R_{P}) can be cleaved, e.g., by a bone phosphatase, such that the Bone Active Portion has one or more activities generally associated with free estradiol, and is allowed to interact with and affect bone.

In some embodiments, the compound can be represented by the following formula, where R_{P} is derived from di-n-butyl phosphate:

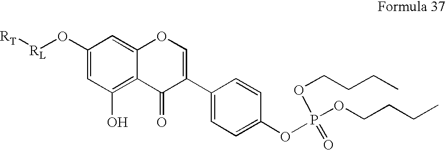

In some embodiments, the compound can be represented by the following formula, where R_{P} is derived from dibenzyl phosphate:

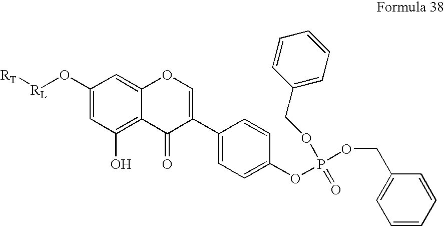

In some embodiments, the compound can be represented by the following formula, where R_{P} is derived from dibenzyl phosphate:

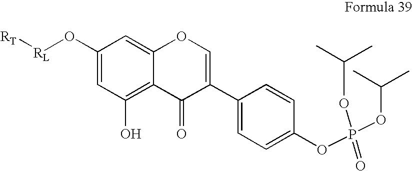

In some embodiments, the compound can be represented by the following formula, where R_{P} is derived from di-tert-butyl phosphate:

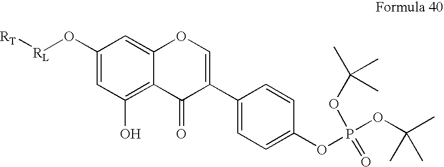

In some embodiments, the compound can be represented by the following formula, where R_{P} is derived from di-2-ethylhexyl phosphate:

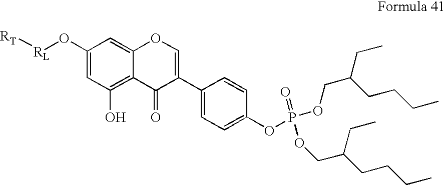

In some embodiments, the compound can be represented by the following formula, where R_{P} is derived from didodecyl phosphate:

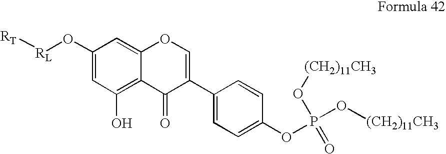

As another non-limiting example, when the Bone Active Portion is derived from genistein and is modified with another exemplary Blocking Group, the compound can be represented by the following formula:

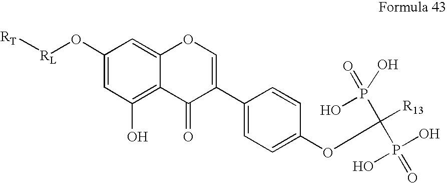

where R_{T} is the Bone Targeting Portion, R_{L} is the Linking Portion, and where R₁₃ can be hydrogen, lower alkyl, alkyl, or aryl. R₁₃ can be selected from the groups as set forth in Table C.

Some exemplary Blocking Groups are thought to have an affinity for bone, which affinity is independent from the affinity provided by the Bone Targeting Portion. Such Blocking Groups having an affinity for bone can be derived from, for example, phosphonates and bisphosphonates.

As noted above, the Bone Active Portion of the compounds can interact with and affect bone, and have a desired effect on bone, i.e., affect a treatment of a condition of interest. Compounds of the presently-disclosed subject matter can affect bone to treat a variety of bone conditions, including those set forth in Tables D and E.

TABLE D
Primary Bone Conditions

| | |
|---|---|
| | Category(ies) |
| | of Bone |
| Primary Bone Condition | Active Agent(s) |

| | | |
|---|---|---|
| Metabolic | Osteoporosis | Anabolic Agent |
| Bone | Paget's Disease | and/or |
| Diseases | Osteogenesis imperfecta | Anti-catabolic |
| (MBD) | Primary hyperparathyroidism | Agent |
| | Fibrous dysplacia | |
| | (McCune-Albright syndrome) | |
| | Osteopetrosis | |
| | Tumor-induced osteomalacia | |
| | Rickets (nutritional, genetic, | |
| | drug-induced) | |
| | Renal osteodystrophy | |
| | Fanconi syndrome | |
| | Hypophosphatasia | |
| Fracture | Fracture resulting from a MBD, | Anabolic |
| | another disease or disorder, or | Agent |
| | an external physical force | |

TABLE E
Primary Conditions, with which another Secondary Bone
Conditions is Associated

| | | Category(ies) |
|---|---|---|
| | Secondary | of |
| | Bone | Bone Active |
| Primary Condition | Condition | Agent(s) |
| Alcoholism | Osteoporosis | Anabolic Agent |
| Anorexia Nervosa (and other | | and/or |
| eating disorders) | | Anti-catabolic |
| Asthma, certain treatment programs | | Agent |
| for; and bone loss associated with | | |
| rheumatoid arthritis | | |
| Autoimmune Diseases, e.g., lupus | | |
| Celiac Disease (Gluten allergy) | | |
| Diabetes | | |
| Inflammatory Bowel Diseases (Crohn's | | |
| Disease, ulcerative colitis) | | |

With reference to Table D, in some embodiments, compounds can be used to treat bone conditions including metabolic bone diseases. In some embodiments, when a metabolic bone disease is being treated, an anti-catabolic effect, an anabolic effect, or a combination thereof is desired. In some embodiments, compounds can be used to treat bone conditions including bone fracture. In some embodiments, when a bone fracture is being treated, an anabolic effect is desired.

With reference to Table E, it can sometimes be desirable to administer to a subject having a primary condition a compound useful for treating a secondary condition. In some embodiments, a subject can be identified as having one or more primary conditions associated with a secondary bone condition that is a metabolic bone disease, such as osteoporosis, as identified in Table E. The subject can then be administered a compound for treating osteoporosis. In some embodiments, such a treatment includes a prophylactic treatment, e.g., arresting or preventing the development of osteoporosis. In some embodiments, an anti-catabolic effect and/or an anabolic effect is desired.

In some embodiments, the compounds can have an anti-catabolic effect on bone. In some embodiments, the compounds can have an anabolic effect on bone. In some embodiments, the compounds can have an anti-catabolic effect and an anabolic effect on bone. In some embodiments, the compounds can be provided in synergistic compositions containing other compounds useful for treating a primary and/or secondary condition.

As used herein, a catabolic effect is an effect that results in a net reduction in bone mass, bone density, and/or bone strength. As used herein, an anti-catabolic effect is an effect that results in a decrease in the magnitude of a catabolic effect. Reduction in bone mass, density, and/or strength can be identified by comparing a first bone measurement (e.g., control or earlier time), to a second bone measurement (e.g., treated or later time). Bone mass, density, and strength can be measured using methods known to those skilled in the art.

As used herein, an anabolic effect is an effect that results in increased bone strength; or increased bone mass or density, and increased bone strength. Increases in bone mass or density, and increases in bone strength provide evidence that net bone formation is being promoted. Increases in bone mass or density, and increases in bone strength can be measured by comparing a first bone measurement (e.g., control or earlier time), to a second bone measurement (e.g., treated or later time.) Bone mass or density can be measured using methods known to those skilled in the art. In some embodiments, requisite increased bone mass or density affected by treatment with a compound of interest is an increase of at least about 5%, at least about 10%, at least about 15%, at least about 20%, at least about 25%, at least about 30%, or at least about 35%, when a first bone measurement and a second bone measurement are compared.

Increased bone strength can be measured by comparing a first bone strength measurement to a second bone strength measurement. In some embodiments, the increased bone strength can be measured by comparing the bone strength of an untreated control (first bone strength measurement), to the bone strength of a bone sample after treatment with a compound of interest (second bone strength measurement). In some embodiments, increased bone strength can be measured by comparing the bone strength of a bone sample before treatment with a compound of interest (first bone strength measurement), to the bone strength of a bone sample after treatment with the compound of interest (second bone strength measurement). Mechanical competence of bone can be determined using methods known to those skilled in the art, for example, a blunt indentation force study, a three point bending to failure test, or a torsional analysis on bone samples from appropriate test subject, e.g., mouse, rat. Percent (%) change in bone strength can be calculated using the following formula:

% change=[(BS₂−BS₁)/BS₁]×100

where BS₁ is the first bone strength measurement, and BS₂ is the second bone strength measurement. An increase in bone strength is identified where the change in bone strength is greater than 0, i.e., a positive % change. In some embodiments, increased bone strength affected by treatment with the compound is an increase in bone strength of at least about 1%. In some embodiments, requisite increased bone strength affected by treatment with the compound is an increase in bone strength of at least about 5%, at least about 10%, at least about 15%, at least about 20%, at least about 25%, at least about 30%, at least about 40%, at least about 50%, at least about 75%, at least about 100%, or at least about 200%, when a first bone strength measurement and a second bone strength measurement are compared.

In some embodiments, for example, where bone strength is being assessed in a human subject, fracture incidence can be recorded, and increased bone strength can be identified where there is a trend of decreased incidence of bone fracture.

Although not necessary to establish anabolic effect, additional information to establish promotion of net bone formation can be obtained. For example, assays can be conducted for certain biomarkers of bone formation (See, e.g., M J Seibel Clin Biochem Rev 26:97 (2005) or “The Use of Biochemical Markers of Bone Turnover in Osteoporosis” by PD Delmas et al. Osteoporosis Int. suppl. 6 S2-17 (2000), which are incorporated herein by these references). As another example, information can be collected as described in Riggs B L, and Parfitt A M, “Drugs Used to Treat Osteoporosis: The Critical Need for a Uniform Nomenclature Based on Their Action on Bone Remodeling,” J. Bone and Mineral Res. 20:2 (2005), which is incorporated herein by this reference. In this regard, in some embodiments, anabolic effect can be identified where a biomarker of bone formation is found in an appropriate test sample, e.g., osteocalcin, collagen type I, as described in the Examples herein. In some embodiments, anabolic effect can be identified pursuant to an assay to evaluate stimulation of bone formation, e.g., calvarial injection, as described in the Examples herein.

### Linking Portion

The Linking Portion (R_{L}) of the compounds connect and separate the Bone Targeting Portion (R_{T}) and a Bone Active Portion. Without wishing to be bound by theory or mechanism, it is believed that the Linking Portion separates the Bone Targeting Portion and the Bone Active Portion to limit steric interference of the Bone Active Portion when interacting with bone.

In some embodiments, the Linking Portion can be described with reference to the following formulas:

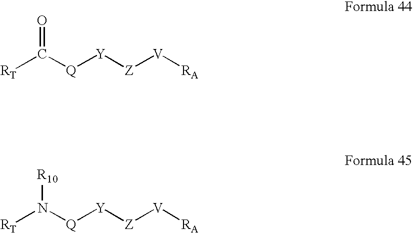

where

R₁₀ is independently hydrogen or lower alkyl;

Q is a straight or branched alkylene group, containing 1 to about 10 carbon atoms on a main chain, and up to a total of about 20 carbon atoms;

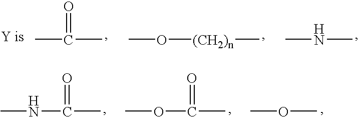

or a chemical bond;

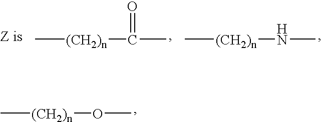

or a chemical bond; V is

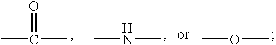

provided Y-Z-V is

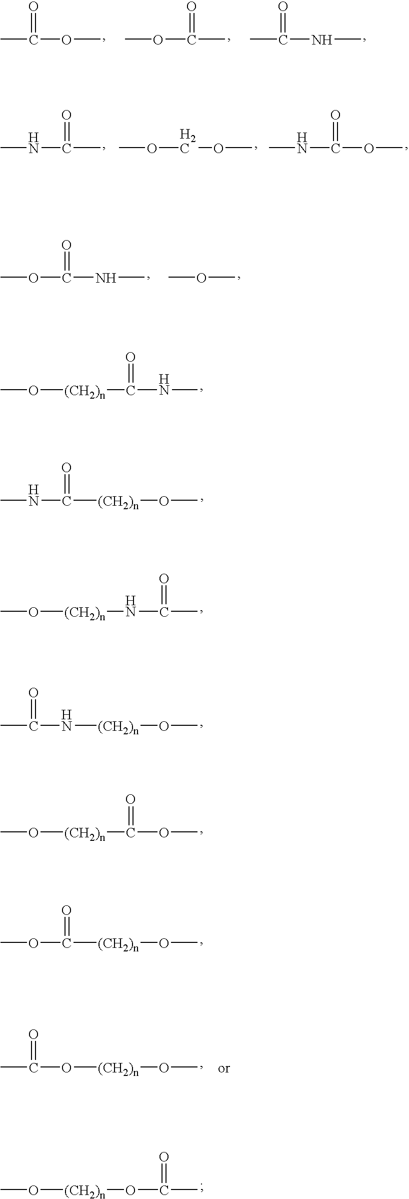

and is n an integer from 0 to 6.

In some embodiments, the Linking Portions of Formulas 44 and 45 can be represented by the following formulas, respectively:

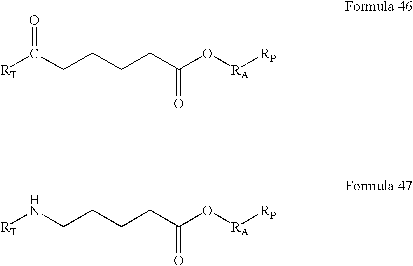

where R₁₀ is H, Q is (CH₂)₄, and YZV is

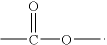

In some embodiments, the Linking Portions of Formulas 44 and 45 can be represented by the following formulas, respectively:

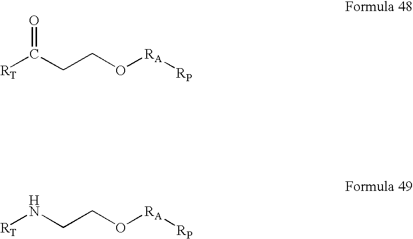

where R₁₀ is H, Q is (CH₂)₂, and YZV is —O—.

In some embodiments, the Linking Portions of Formulas 44 and 45 can be represented by the following formulas, respectively:

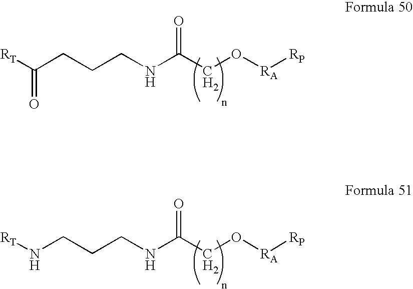

where R₁₀ is H, Q is (CH₂)₃, and YZV is

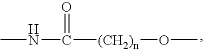

where n is 0 to about 4.

In some embodiments, the Linking Portions of Formulas 44 and 45 can be represented by the following formulas, respectively:

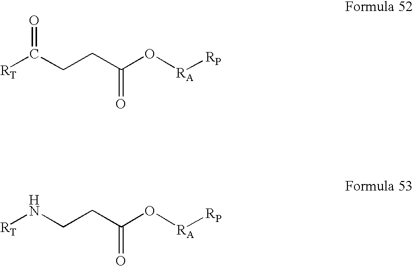

where R₁₀ is H, Q is (CH₂)₂, and YZV is

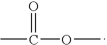

When the Linking Portion of Formula 44 is selected, the Linking Portion is connected to the Bone Targeting Portion (R_{T}) in the place of R₁ or R₂, as represented by the following formula:

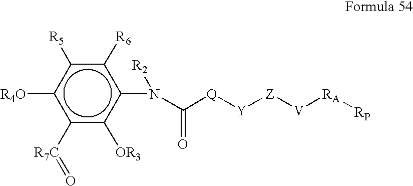

where the Linking Portion is connected to the Bone Targeting Portion (R_{T}) in the place of R₁.

The Bone Active Portion (R_{A}) that is derived from an estrogenic agent can be modified relative to the estrogenic agent as is necessary to be connected to the remainder of the compound, while maintaining some or all of the activity associated with the estrogenic agent, or while obtaining an enhanced activity relative to the estrogenic agent For example, a Bone Active Portion derived from an estrogenic agent after being linked to the compound can have the structure of the estrogenic agent, less a leaving group, e.g., hydroxyl group, amino group, or other group present on the estrogenic agent, or including a connecting group, as will be apparent to one of ordinary skill in the art. In some embodiments, where the Bone Active Portion has the structure of an estrogenic agent, less a leaving group, the estrogenic agent can donate a portion of the leaving group to the V segment of the Linking Portion, creating a connection, e.g., ether, amide, ester) between the V segment of the Linking Portion and the Bone Active Portion (R_{A}). For example, in some embodiments, the Bone Active Portion can be derived from estradiol, which is less a hydroxy group, and where the oxygen of the hydroxy group is donated to the V segment of the Linking Portion, as represented by the following formula:

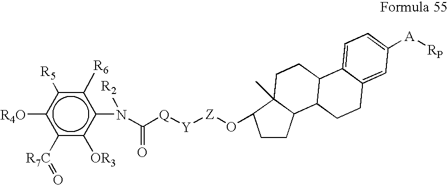

In some embodiments, the compound can be represented by the following formula:

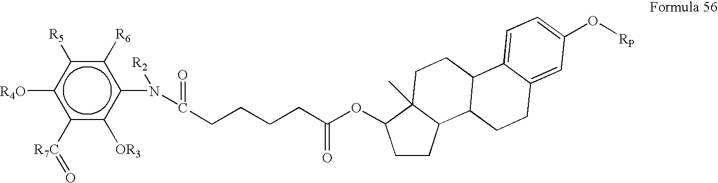

where Q is (CH₂)₄, YZV is

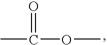

### and A is O.

In some embodiments, the compound can be represented by the following formula:

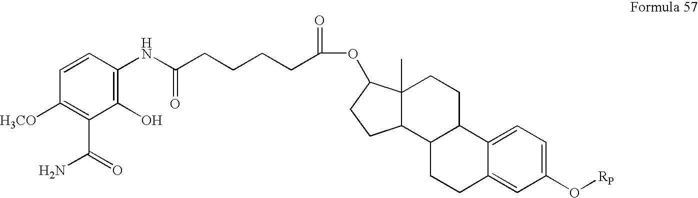

where Q is (CH₂)₄, YZV is

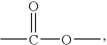

A is O, and where R₂, R₃, R₅, and R₆ are each H, R₄ is CH₃, and R₇ is NH₂.

In some embodiments, the compound can be represented by the following formula:

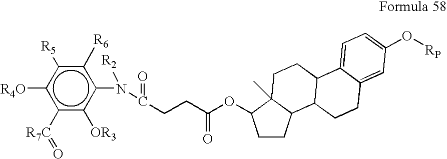

where Q is (CH₂)₂, YZV is

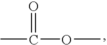

### and A is O.

In some embodiments, the compound can be represented by the following formula:

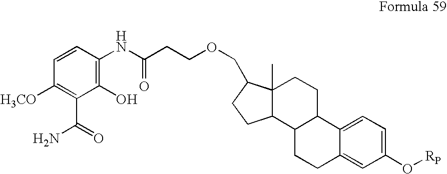

where Q is (CH₂)₂, YZV is

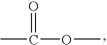

A is O, and where R₂, R₃, R₅, and R₆ are each H, R₄ is CH₃, and R₇ is NH₂.

In some embodiments, the compound can be represented by the following formula:

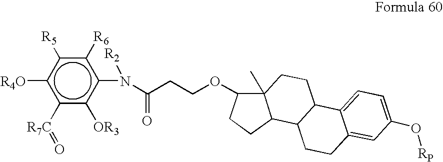

where Q is (CH₂)₂, YZV is —O—, and A is O.

In some embodiments, the compound can be represented by the following formula:

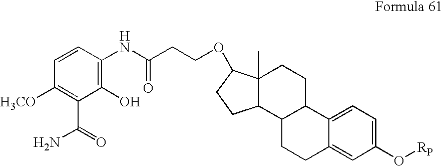

where Q is (CH₂)₂, YZV is —O—, A is O, and where R₂, R₃, R₅, and R₆ are each H, R₄ is CH₃, and R₇ is NH₂.

In some embodiments, the compound can be represented by the following formula:

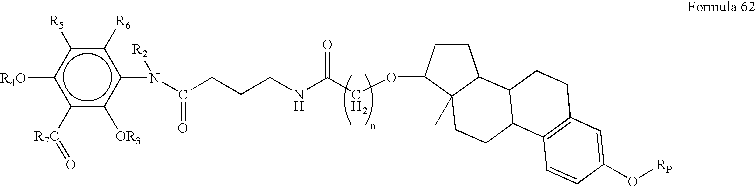

where Q is (CH₂)₂, YZV is

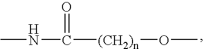

where n is 0 to about 4, and A is O.

In some embodiments, the compound can be represented by the following formula:

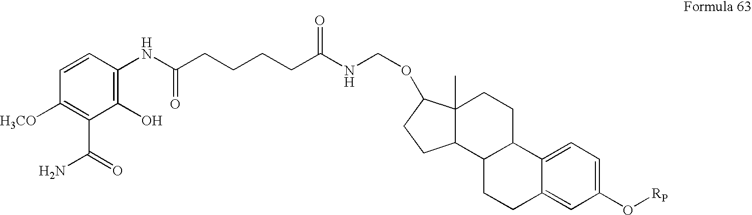

where Q is (CH₂)₂, YZV is

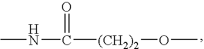

A is O, and where R₂, R₃, R₅, and R₆ are each H, R₄ is CH₃, and R₇ is NH₂.

For another non-limiting example, the compound can be represented by the following formula:

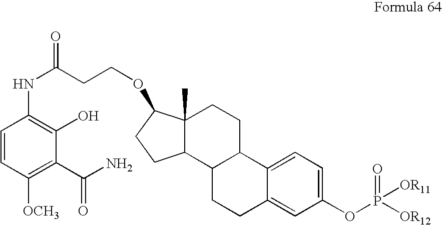

where Q is (CH₂)₂, YZV is —O—, A is O, and where R₂, R₃, R₅, and R₆ are each H, R₄ is CH₃, and R₇ is NH₂.

In some embodiments, the compound can be represented by the following formula, where R_{P} is derived from phosphoric acid:

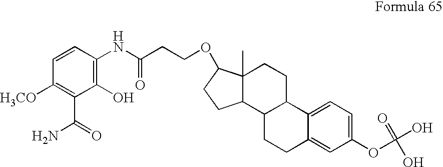

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

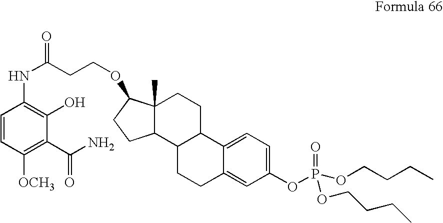

In some embodiments, the compound can be represented by the following formula, where R_{P} is derived from dibenzyl phosphate:

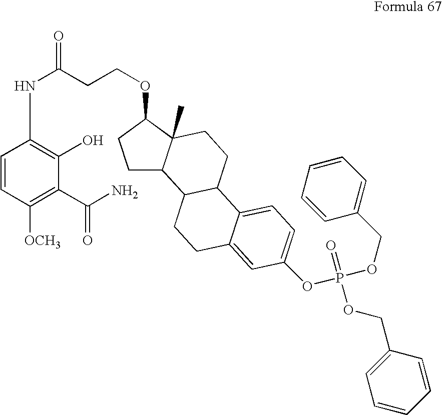

In some embodiments, the compound can be represented by the following formula, where R_{P} is derived from diisopropyl phosphate:

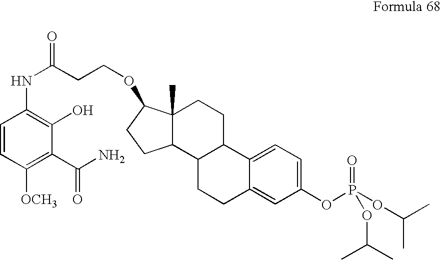

In some embodiments, the compound can be represented by the following formula, where R_{P} is derived from di-tert-butyl phosphate:

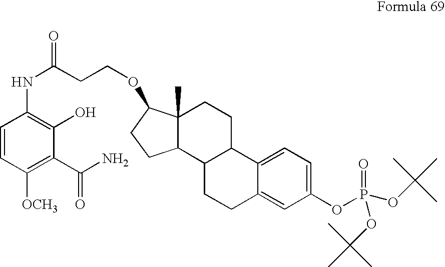

In some embodiments, the compound can be represented by the following formula, where R_{P} is derived from di-2-ethylhexyl phosphate:

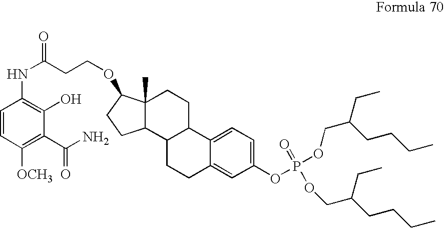

In some embodiments, the compound can be represented by the following formula, where R_{P} is derived from didodecyl phosphate:

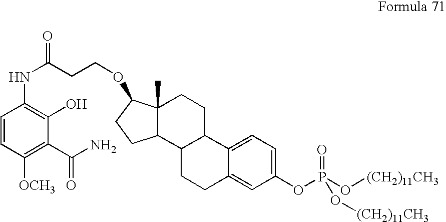

As noted above, the Bone Active Portion can be derived from an estrogenic agent. Although examples of compounds, such as the examples set forth in Formulas 55-71, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

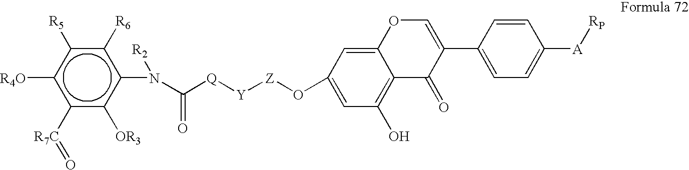

When the Linking Portion of Formula 45 is selected, the Linking Portion is connected to the Bone Targeting Portion (R_{T}) in the place of R₇, as represented by the following formula:

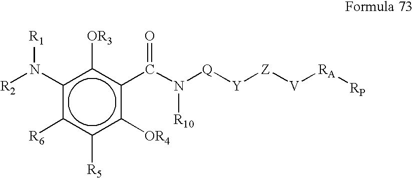

The Bone Active Portion (R_{A}) that is derived from an estrogenic agent can be modified relative to the estrogenic agent as is necessary to be connected to the remainder of the compound, while maintaining some or all of the activity associated with the estrogenic agent, or while obtaining enhanced activity relative to the estrogenic agent. For example, a Bone Active Portion derived from an estrogenic agent after being linked to the compound can have the structure of the estrogenic agent, less a leaving group, e.g., hydroxyl group, amino group, or other group present on the estrogenic agent, or including a connecting group, as will be apparent to one of ordinary skill in the art. In some embodiments, where the Bone Active Portion has the structure of an estrogenic agent, less a leaving group, the estrogenic agent can donate a portion of the leaving group to the V segment of the Linking Portion, creating a connection, e.g., ether, amide, ester) between the V segment of the Linking Portion and the Bone Active Portion (R_{A}). For example, in some embodiments, the Bone Active Portion can be derived from estradiol, which is less a hydroxy group, and where the oxygen of the hydroxy group is donated to the V segment of the Linking Portion, as represented by the following formula:

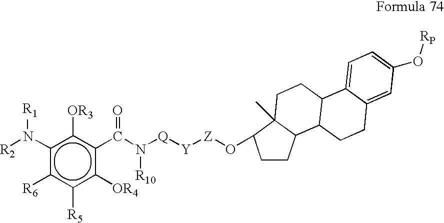

In some embodiments, the compound can be represented by the following formula:

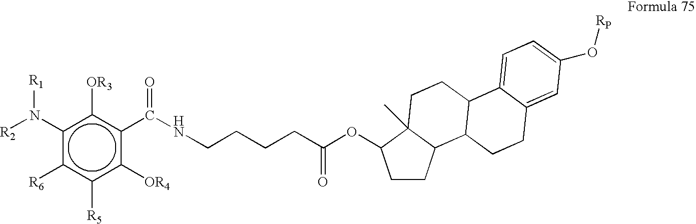

where Q is (CH₂)₄, YZV is

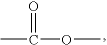

### and A is O.

In some embodiments, the compound can be represented by the following formula:

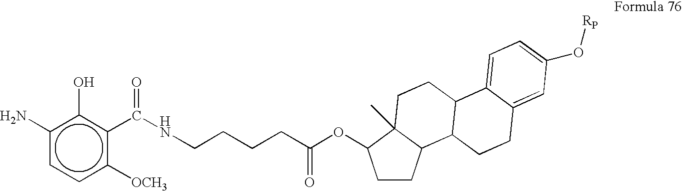

where Q is (CH₂)₄, YZV is

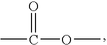

A is O, and where R₁, R₂, R₃, R₅, and R₆ are each H, and R₄ is CH₃.

In some embodiments, the compound can be represented by the following formula:

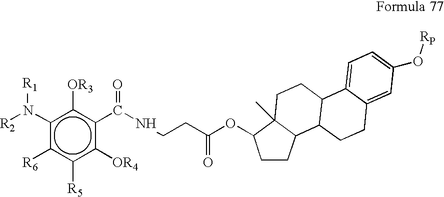

where Q is (CH₂)₂, YZV is

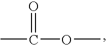

### and A is O.

In some embodiments, the compound can be represented by the following formula:

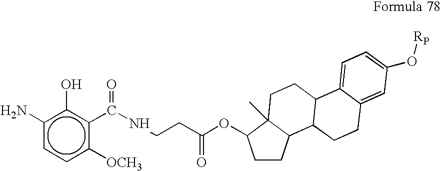

where Q is (CH₂)₂, YZV is

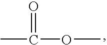

A is O, and where R₁, R₂, R₃, R₅, and R₆ are each H, and R₄ is CH₃.

In some embodiments, the compound can be represented by the following formula:

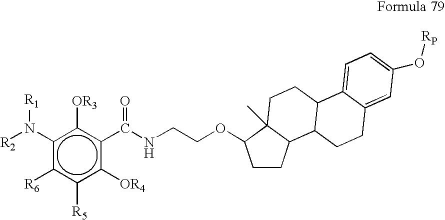

where Q is (CH₂)₂, YZV is —O—, and A is O.

In some embodiments, the compound can be represented by the following formula:

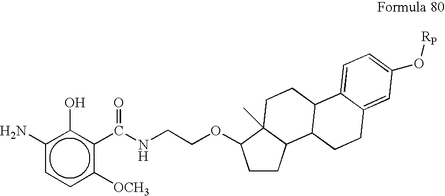

where Q is (CH₂)₂, YZV is —O—, A is O, and where R₁, R₂, R₃, R₄, R₅, and R₆ are each H, and R₄ is CH₃.

In some embodiments, the compound can be represented by the following formula:

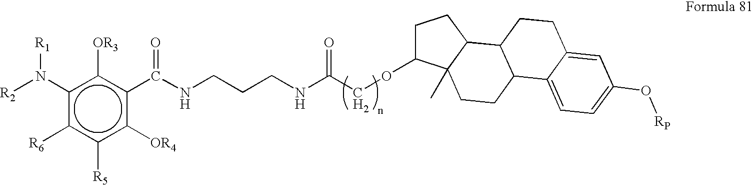

where Q is (CH₂)₂, YZV is

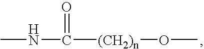

where n is 0 to about 4, and A is O.

In some embodiments, the compound can be represented by the following formula:

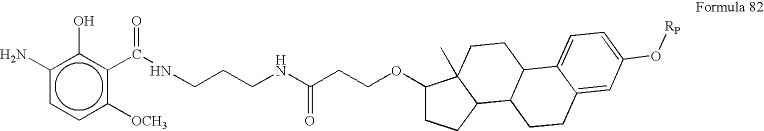

where Q is (CH₂)₂, YZV is

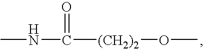

A is O, and where R₁, R₂, R₃, R₅, and R₆ are each H, and R₄ is CH₃.

For another non-limiting example, the compound can be represented by the following formula:

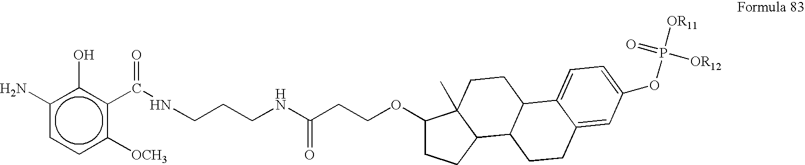

where Q is (CH₂)₂, YZV is

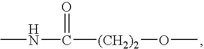

and A is O; where R₁, R₂, R₃, R₅, and R₆ are each H, and R₄ is CH₃. When R_{P} is derived from phosphoric acid, the compound can in some embodiments be provided as a salt, ester, or ether thereof.

In some embodiments, the compound can be represented by the following formula, where R_{P} is derived from phosphoric acid:

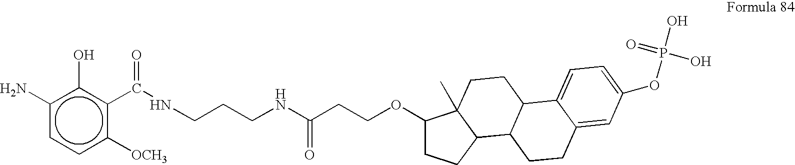

In some embodiments, the compound can be represented by the following formula, where R_{P} is derived from di-n-butyl phosphate:

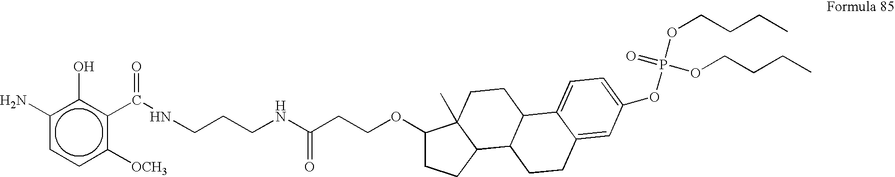

In some embodiments, the compound can be represented by the following formula, where R_{P} is derived from dibenzyl phosphate:

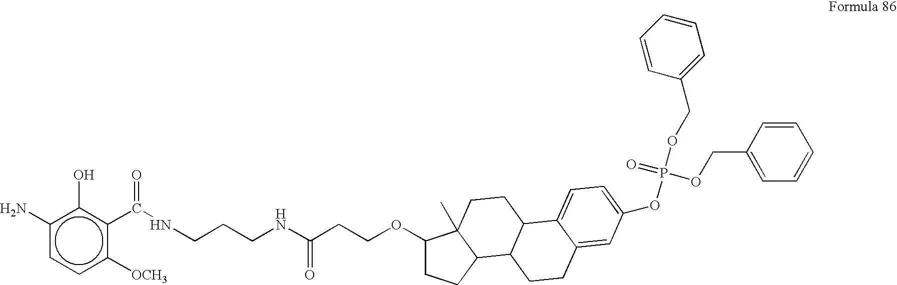

In some embodiments, the compound can be represented by the following formula, where R_{P} is derived from diisopropyl phosphate:

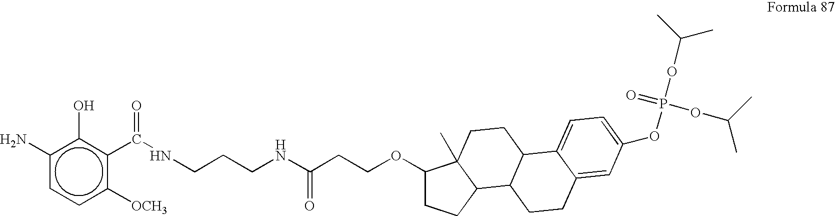

In some embodiments, the compound can be represented by the following formula, where R_{P} is derived from di-tert-butyl phosphate:

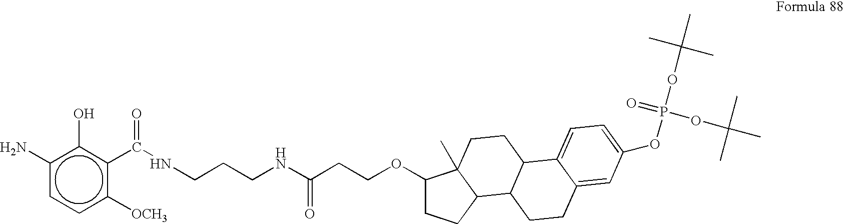

In some embodiments, the compound can be represented by the following formula, where R_{P} is derived from di-2-ethylhexyl phosphate:

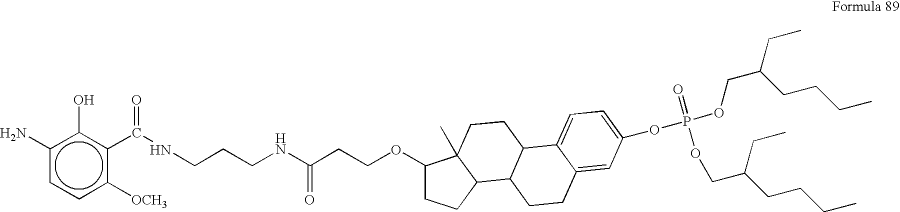

In some embodiments, the compound can be represented by the following formula, where R_{P} is derived from didodecyl phosphate:

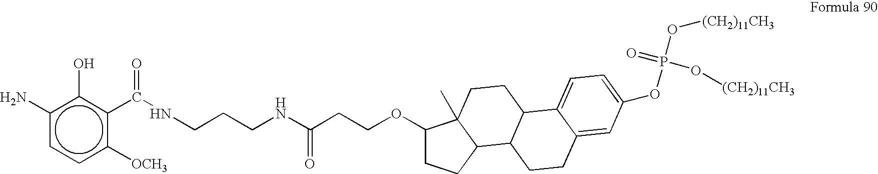

As noted above, the Bone Active Portion can be derived from an estrogenic agent. Although examples of compounds, such as the examples set forth in Formulas 73-90, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

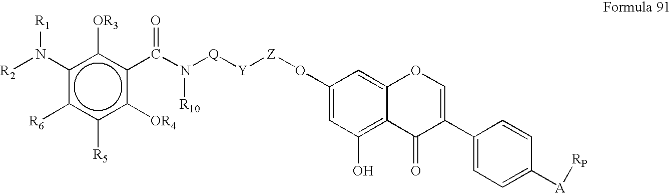

In some embodiments, the Linking Portion can be described with reference to the following formulas:

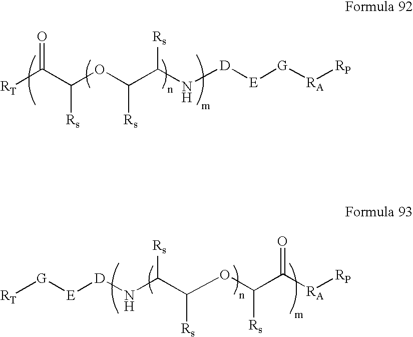

where the Linking Portion extends between R_{T} and R_{A}.

The length of the linking portion of the compounds of the presently-disclosed subject matter can vary, depending on the embodiment of the presently-disclosed subject matter. In this regard, m can be 1 to about 3, and n can be 1 to about 4. For example, when m=1 and n=2, the compounds can be represented by the following formulas:

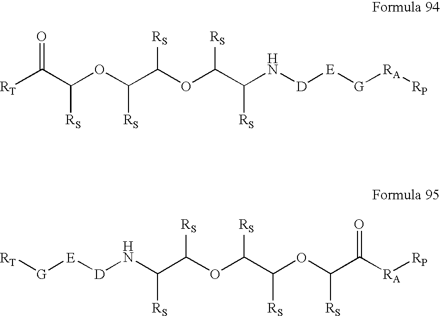

For another example, when m=1 and n=3, the compounds can be represented by the following formulas:

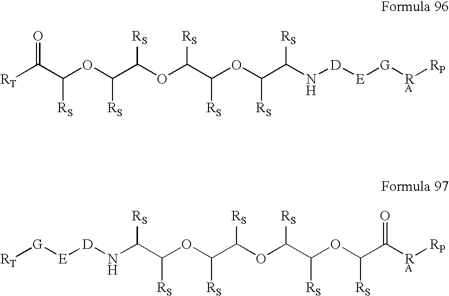

When m>1, multiple n-groups are provided. When multiple n-groups are provided, each n is independently 1 to about 4. For example, when m=3, three n-groups are provided, n′, n″, and n′″, which are each independently 1 to about 4. For another example, when m=2, two n-groups are provided, n′ and n″, as shown in the following formulas:

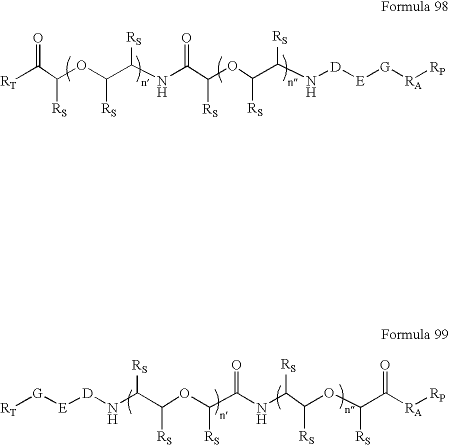

where n′ and n″ are each independently 1 to about 4. For example, when m=2, n′=1, and n″=2, compounds according to the following compounds are provided:

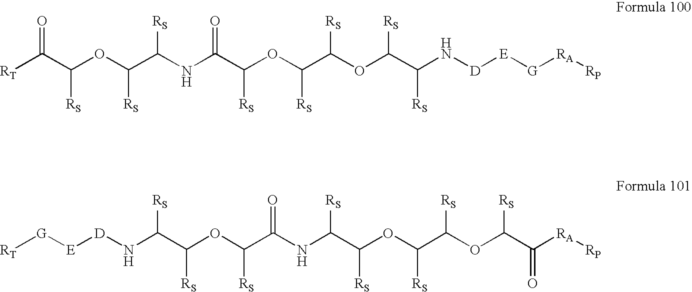

The groups of the linking portion identified as R_{S} can be hydrogen, hydroxy, or lower alkyl. It is noted that R_{S} should not be hydroxy when bound to a carbon atom that is also bound to another heteroatom, which would form an unstable hemiacetal or hemiaminal.

In some embodiments, every R_{S} group can be hydrogen, as shown in the following formula, where m is 1 and n is 2:

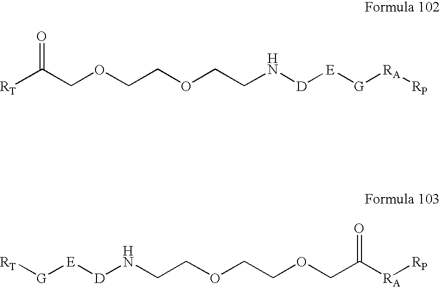

The groups of the linking portion identified as D, E, and G are as follows. D and G are independently selected from: covalent bond;

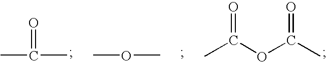

or other functional groups capable of reacting with an amine, less a leaving group. That is to say, for example, an acyl halide (X—C═O) is a functional group capable of reacting with an amine, and a carbonyl

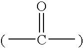

is an acyl halide, less a halogen atom (X). E is selected from: covalent bond; —(CTr)-, where T is H, OH, or lower alkyl, and m=1 to about 8; and —(C)ᵣ—, where m=2 to about 8, and where the carbons are unsaturated or partially saturated with H.

In some embodiments, the compound can be represented by the following formula:

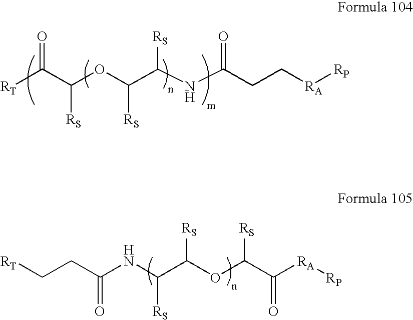

where D is

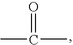

E is —(CH₂)—, and G is a covalent bond.

D, E, and G can be selected, for example, based on the portion of the compound to which the linking group will be bound. For example, when the Linking Portion of Formula 67 is used, the -D-E-G- segment of the Linking Portion is adjacent the Bone Targeting Portion of the compound. The Linking Portion can be connected to the Bone Targeting Portion (R_{T}) in the place of R₁, as shown in the following formula:

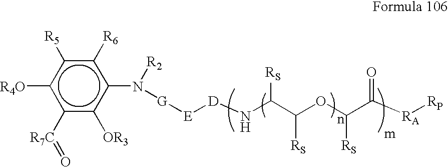

When the Linking Portion of Formula 93 is connected to the Bone Targeting Portion (R_{T}) in the place of R₁, as will be understood by those skilled in the art, it can be beneficial for G to be selected to be a functional group capable of reacting with an amine, less a leaving group, for example,

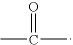

In this regard, in some embodiments, the compound can be represented by the following formula:

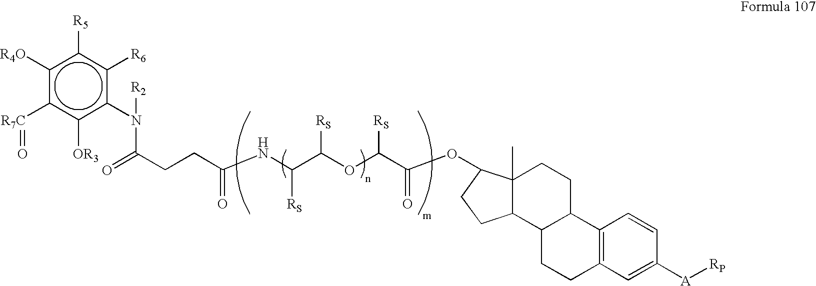

where the Linking Portion is connected to the Bone Targeting Portion (R_{T}) in the place of R₁; where D and G are each

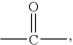

where E is —(CH₂)₂—, and where R_{A} is a Bone Active Portion that is derived from estradiol. The Bone Active Portion derived from estradiol is estradiol less a hydrogen, allowing the Bone Active Portion to be connected to the remainder of the compound, while maintaining one or more activities generally associated with free estradiol, or while obtaining enhanced activity relative to estradiol.

In some embodiments, the compound can be represented by the following formula:

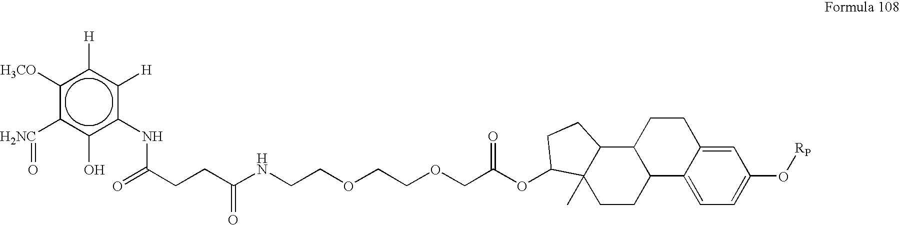

where: m is 1; n is 2; each R₅ is hydrogen; R₂, R₃, R₅, R₆ are each H; R₄ is methyl; and R₇ is amino.

For another non-limiting example, the compound can be represented by the following formula:

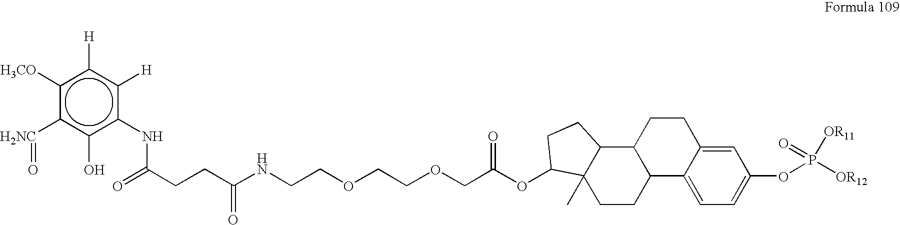

In some embodiments, the compound can be represented by the following formula, where R_{P} is derived from phosphoric acid:

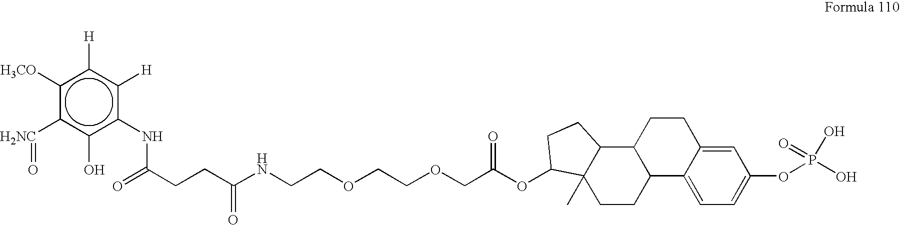

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

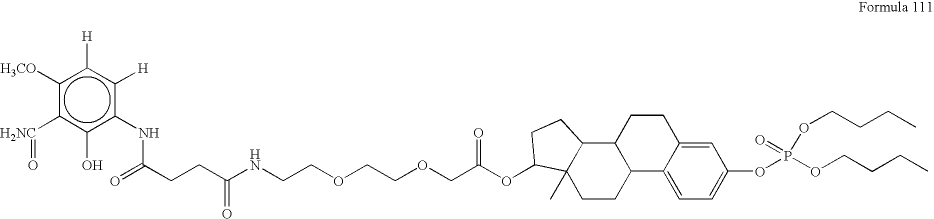

In some embodiments, the compound can be represented by the following formula, where R_{P} is derived from dibenzyl phosphate:

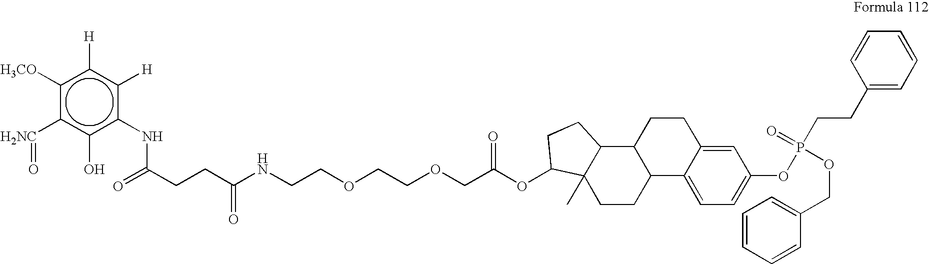

In some embodiments, the compound can be represented by the following formula, where R_{P} is derived from diisopropyl phosphate:

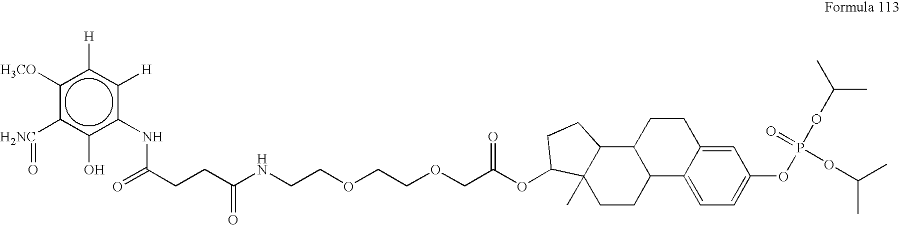

In some embodiments, the compound can be represented by the following formula, where R_{P} is derived from di-tert-butyl phosphate:

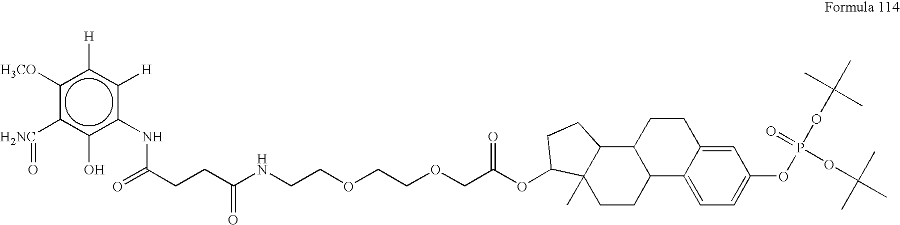

In some embodiments, the compound can be represented by the following formula, where R_{P} is derived from di-2-ethylhexyl phosphate:

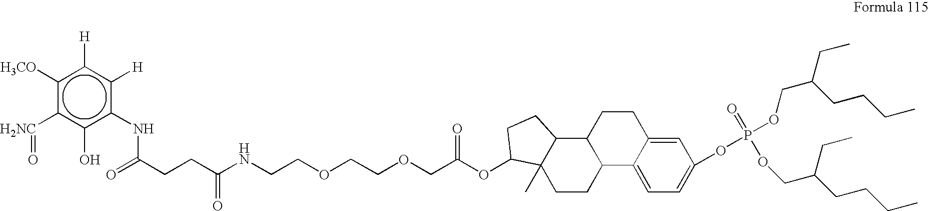

In some embodiments, the compound can be represented by the following formula, where R_{P} is derived from didodecyl phosphate:

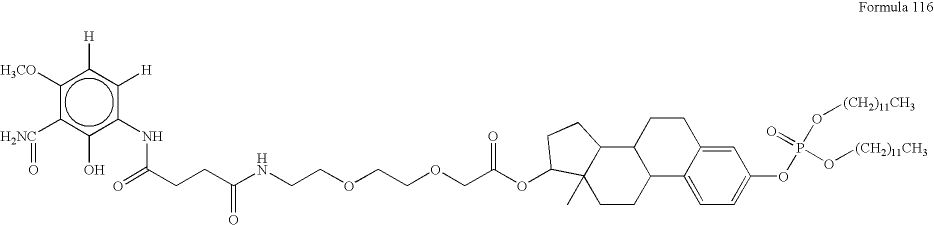

When the Linking Portion of Formula 93 is used, it can also be connected to the Bone Targeting Portion (R_{T}) in the place of R₄, as shown in the following formula:

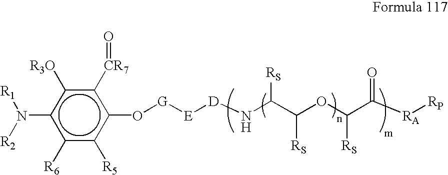

When the Linking Portion of Formula 93 is connected to the Bone Targeting Portion (R_{T}) in the place of R₄, as will be understood by those skilled in the art, the following can be beneficial: G is a covalent bond; and D is a functional group capable of reacting with an amine, less a leaving group. In some embodiments, the compound can be represented by the following formula:

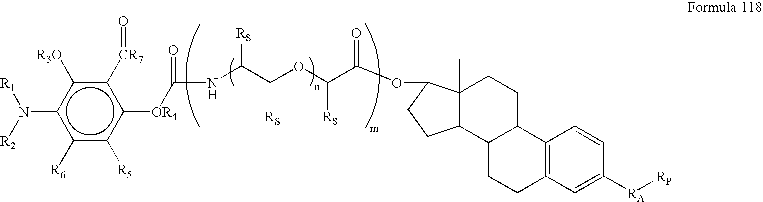

where R_{A} is derived from a bone active agent, estradiol.

In some embodiments, the compound can be represented by the following formula:

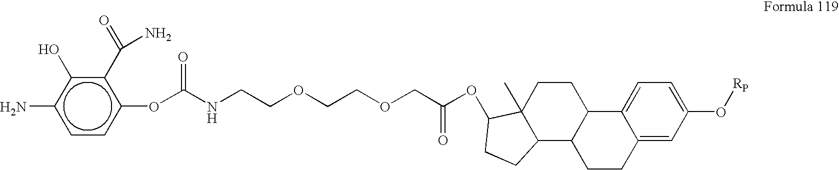

where: m is 1; n is 2; each R₅ is hydrogen; and R₁, R₂, R₃, R₅, R₆, R₇ are each H.

In some embodiments, the compound can be represented by the following formula:

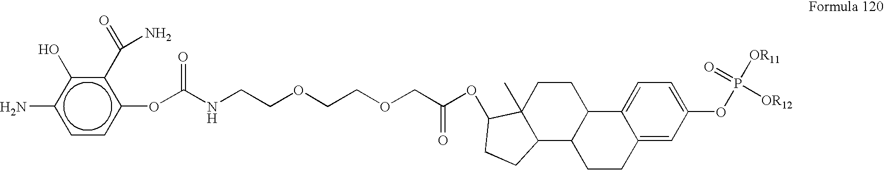

where: m is 1; n is 2; each R₅ is hydrogen; R₁, R₂, R₃, R₅, R₆, R₇ are each H; and A is O.

In some embodiments, the compound can be represented by the following formula, where R_{P} is derived from phosphoric acid:

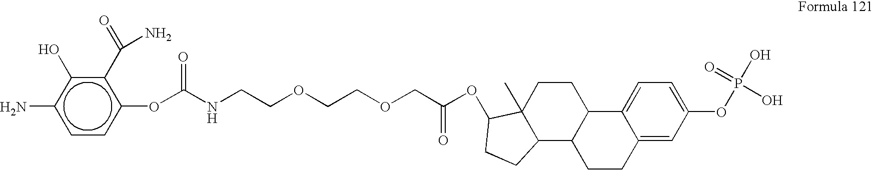

In some embodiments, the compound can be represented by the following formula, where R_{P} is derived from di-n-butyl phosphate:

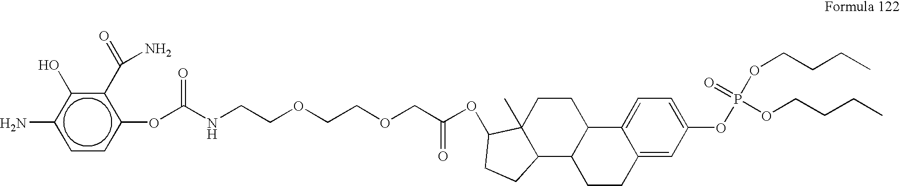

In some embodiments, the compound can be represented by the following formula, where R_{P} is derived from dibenzyl phosphate:

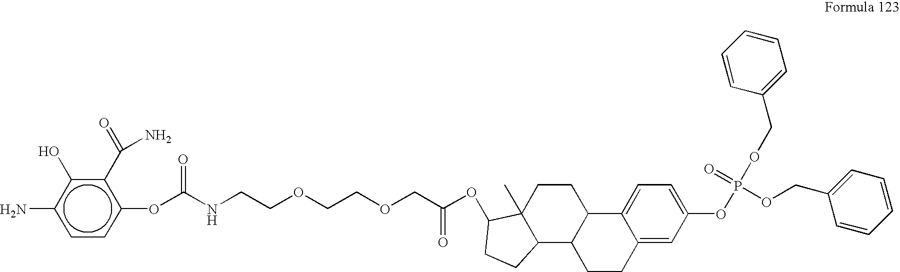

In some embodiments, the compound can be represented by the following formula, where R_{P} is derived from diisopropyl phosphate:

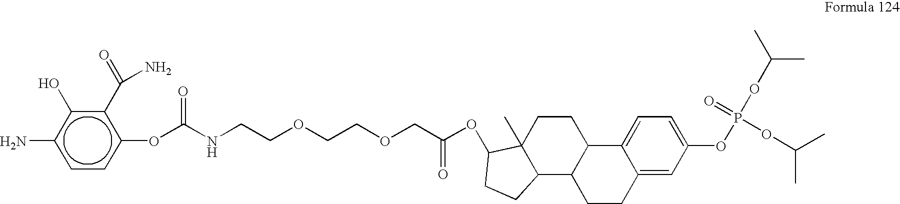

In some embodiments, the compound can be represented by the following formula, where R_{P} is derived from di-tert-butyl phosphate:

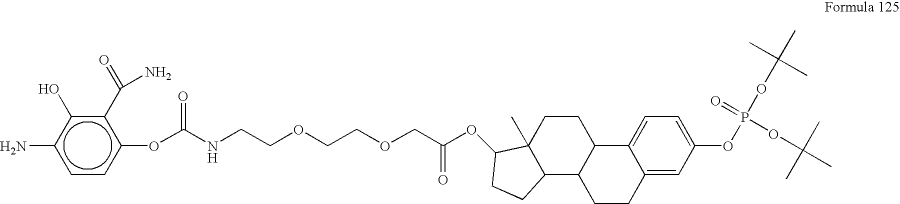

In some embodiments, the compound can be represented by the following formula, where R_{P} is derived from di-2-ethylhexyl phosphate:

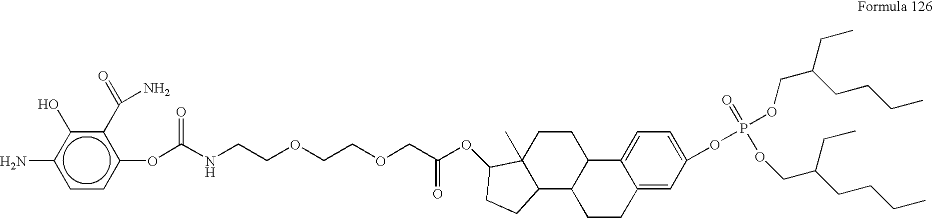

In some embodiments, the compound can be represented by the following formula, where R_{P} is derived from didodecyl phosphate:

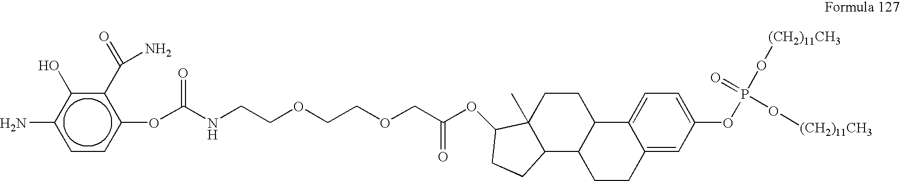

As noted above, the Bone Active Portion can be derived from an estrogenic agent. Although examples of compounds, such as the examples set forth in Formulas 109-127, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

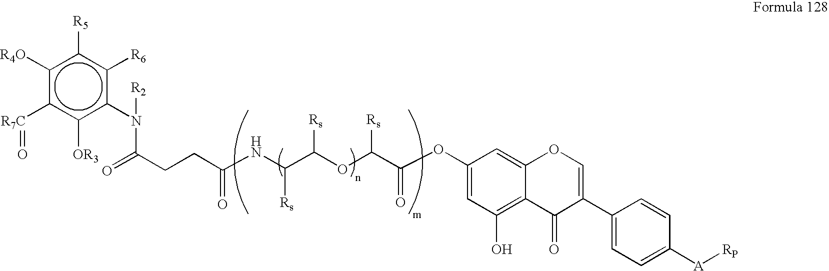

With reference again to the -D-E-G- segment of the Linking Portion, when the Linking Portion of Formula 92 is used, the -D-E-G- segment of the Linking Portion is adjacent the R_{A} portion of the compounds, as shown in the following formula, where the Linking Portion is connected to the Bone Targeting Portion in the place of R₁:

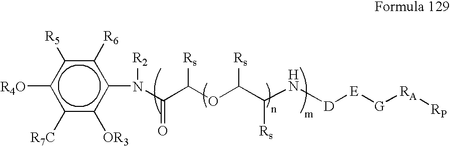

Because the -D-E-G- segment is positioned adjacent the third unit, R_{A}, it can be beneficial to select -D-E-G- based on the selected third unit, R_{A}. For example, when R_{A} is a Bone Active Portion, it is contemplated that the Linker Portion can be bound to the Bone Active Portion to minimize the susceptibility to hydrolysis, e.g., ester, ureido, ether, linkage, to increase the bioavailablity of the compound. That is to say, if susceptibility to hydrolysis is minimized, the compound can be delivered to and affect bone, before the Bone Active Portion can be cleaved from the compound.

In some embodiments, R_{A} can be derived from a bone active agent, estradiol. As will be understood by those skilled in the art, in such cases, it can be beneficial in some embodiments for D to be

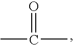

for E to be a group other than a covalent bond, and for G to be a covalent bond. In this regard, in some embodiments, the compound can be represented by the following formula:

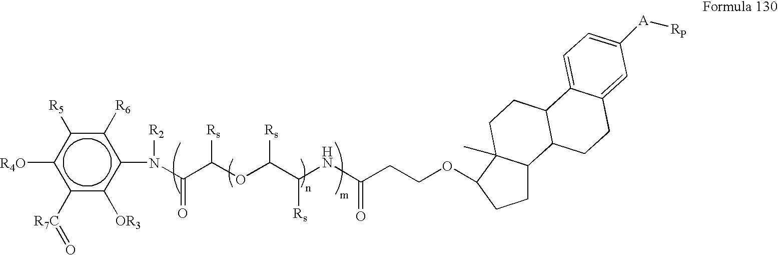

where D is

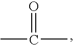

E is —(CH₂)₂—, and G is a covalent bond.

When the Linker Portion of Formula 66 is selected, the -D-E-G- segment is positioned adjacent the Bone Active Portion, R_{A}, and it can be beneficial to select -D-E-G-based on the selected third unit, R_{A}. For example, it is contemplated that the Linker Portion can be bound to the Bone Active Portion to minimize the susceptibility to hydrolysis, e.g., ester, ureido, ether, linkage, to increase the bioavailablity of the compound. That is to say, if susceptibility to hydrolysis is minimized, the compound can be delivered to and affect bone, before the Bone Active Portion can be cleaved from the compound. In some embodiments, R_{A} can be derived from estradiol. As will be understood by those skilled in the art, in such cases, it can be beneficial in some embodiments for D to be

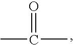

for E to be a group other than a covalent bond, and for G to be a covalent bond. In this regard, in some embodiments, the compound can be represented by the following formula:

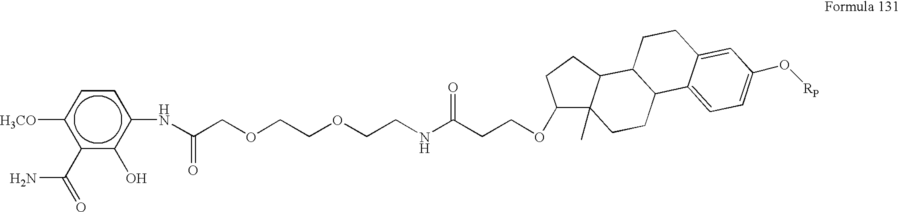

where: n is 2; each R₅ is H; R₂, R₃, R₅, R₆ are each H; R₄ is methyl; R₇ is amino; D is

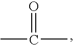

E is —(CH₂)₂—, and G is a covalent bond.

In some embodiments, the compound can be represented by the following formula:

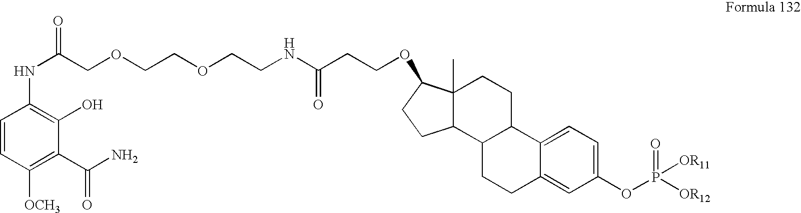

where: n is 2; each R₅ is H; R₂, R₃, R₅, R₆ are each H; R₄ is methyl; R₇ is amino; D is

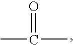

E is —(CH₂)₂—, and G is a covalent bond.

In some embodiments, the compound can be represented by the following formula, where R_{P} is derived from phosphoric acid:

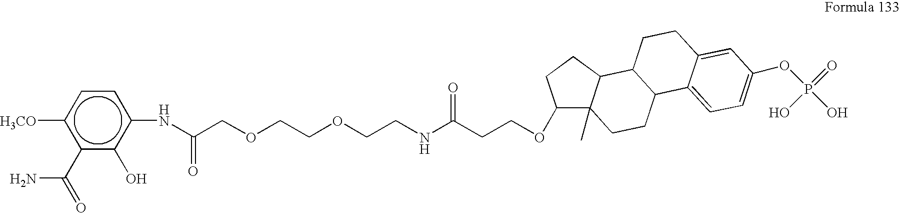

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

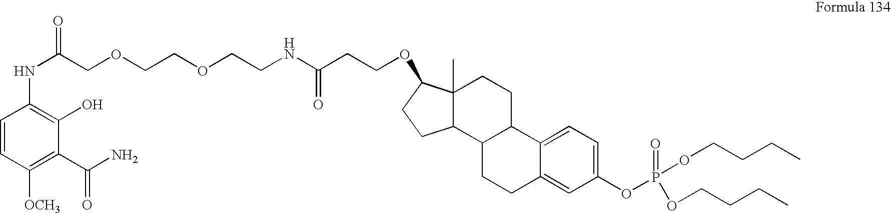

In some embodiments, the compound can be represented by the following formula, where R_{P} is derived from dibenzyl phosphate:

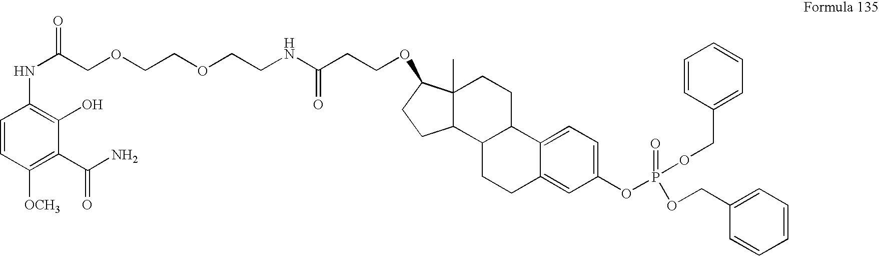

In some embodiments, the compound can be represented by the following formula, where R_{P} is derived from diisopropyl phosphate:

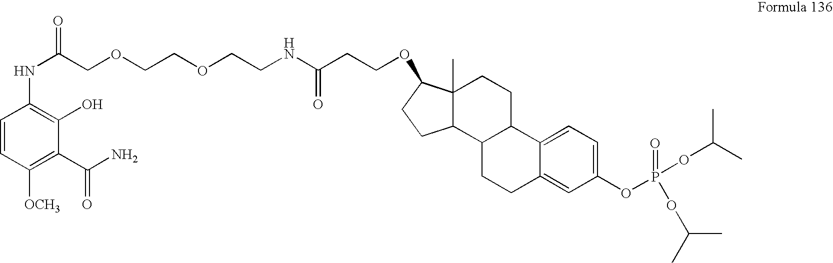

In some embodiments, the compound can be represented by the following formula, where R_{P} is derived from di-tert-butyl phosphate:

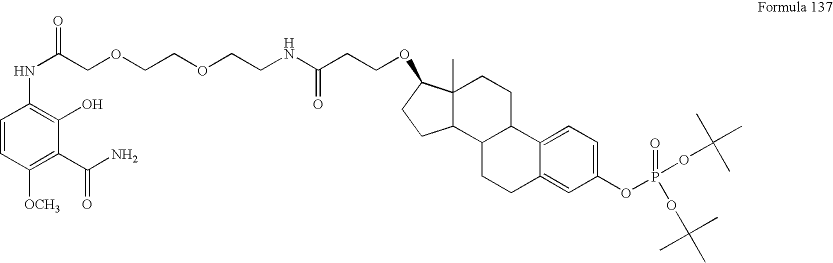

In some embodiments, the compound can be represented by the following formula, where R_{P} is derived from di-2-ethylhexyl phosphate:

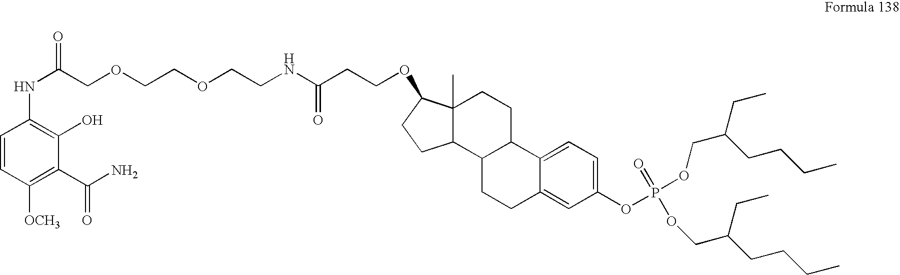

In some embodiments, the compound can be represented by the following formula, where R_{P} is derived from didodecyl phosphate:

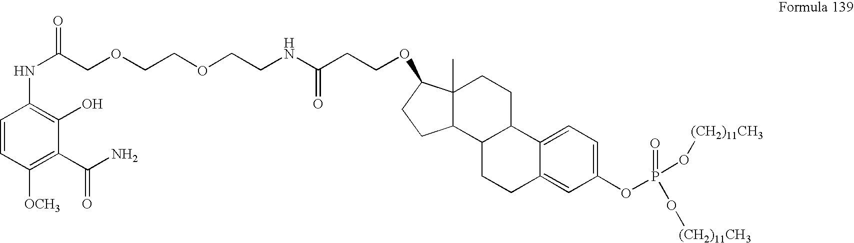

As noted above, the Bone Active Portion can be derived from an estrogenic agent. Although examples of compounds, such as the examples set forth in Formulas 130-139, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

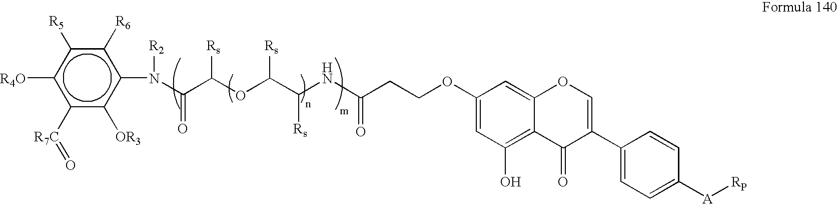

When the Linking Portion of Formula 92 is used, the Linking Portion can be connected to the Bone Targeting Portion in the place of R₄, as represented by the following formula:

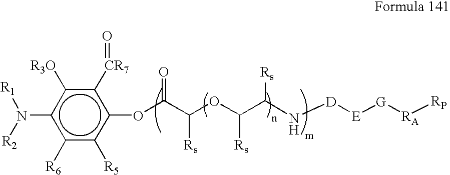

Because the -D-E-G- segment is positioned adjacent the third unit, R_{A}, it can be beneficial to select -D-E-G- based on the selected third unit, R_{A}. For example, when R_{A} is a Bone Active Portion, it is contemplated that the Linker Portion can be bound to the Bone Active Portion to minimize the susceptibility to hydrolysis, e.g., ester, ureido, ether, linkage, to increase the bioavailablity of the compound. That is to say, if susceptibility to hydrolysis is minimized, the compound can be delivered to and affect bone, before the Bone Active Portion can be cleaved from the compound.

In some embodiments, R_{A} can be derived from a bone active agent, estradiol. As will be understood by those skilled in the art, in such cases, it can be beneficial in some embodiments for D to be

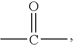

for E to be a group other than a covalent bond, and for G to be a covalent bond. In this regard, in some embodiments, the compound can be represented by the following formula:

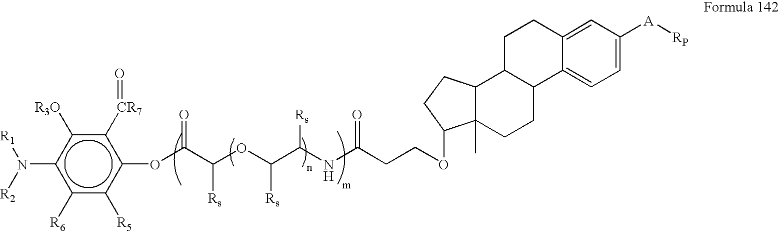

where D is

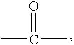

E is —(CH₂)₂—, and G is a covalent bond.

When the Linker Portion of Formula 92 is selected, the -D-E-G- segment is positioned adjacent the Bone Active Portion, R_{A}, and it can be beneficial to select -D-E-G-based on the selected third unit, R_{A}. For example, it is contemplated that the Linker Portion can be bound to the Bone Active Portion to minimize the susceptibility to hydrolysis, e.g., ester, ureido, ether, linkage, to increase the bioavailablity of the compound. That is to say, if susceptibility to hydrolysis is minimized, the compound can be delivered to and affect bone, before the Bone Active Portion can be cleaved from the compound. In some embodiments, R_{A} can be derived from estradiol. As will be understood by those skilled in the art, in such cases, it can be beneficial in some embodiments for D to be for

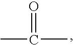

E to be a group other than a covalent bond, and for G to be a covalent bond. In this regard, in some embodiments, the compound can be represented by the following formula:

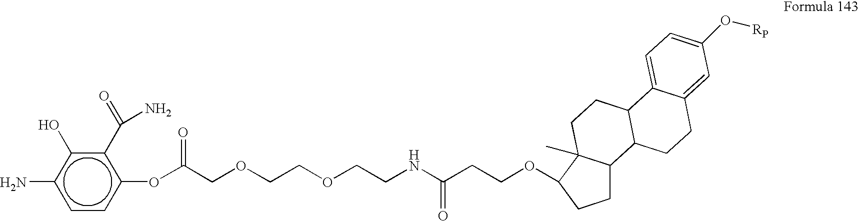

where: n is 2; each R₅ is H; R₁, R₂, R₃, R₅, R₆ are each H; R₇ is amino; D is

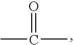

E is —(CH₂)₂—, G is a covalent bond, and M is O.

In some embodiments, the compound can be represented by the following formula:

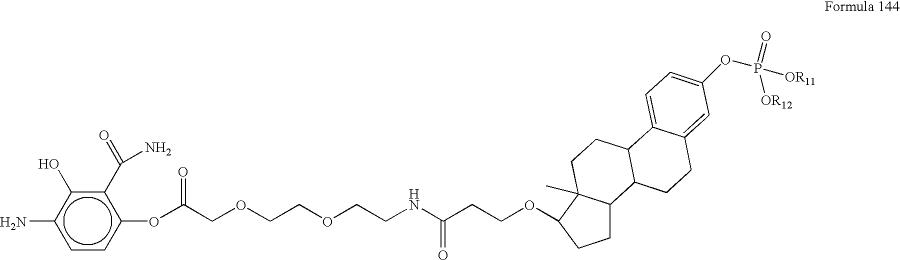

In some embodiments, the compound can be represented by the following formula, where R_{P} is derived from phosphoric acid:

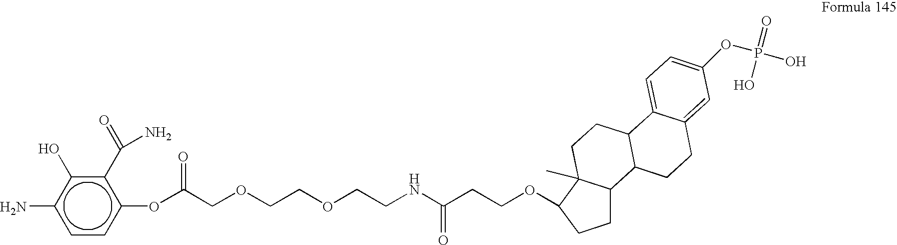

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

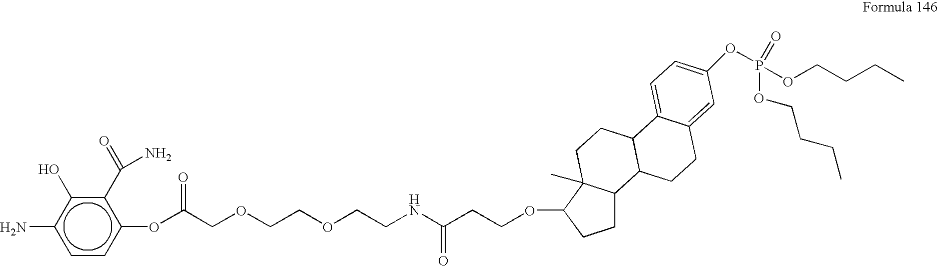

In some embodiments, the compound can be represented by the following formula, where R_{P} is derived from dibenzyl phosphate:

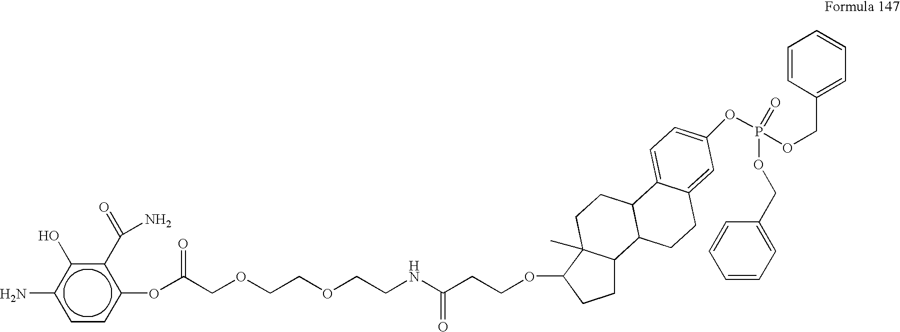

In some embodiments, the compound can be represented by the following formula, where R_{P} is derived from diisopropyl phosphate:

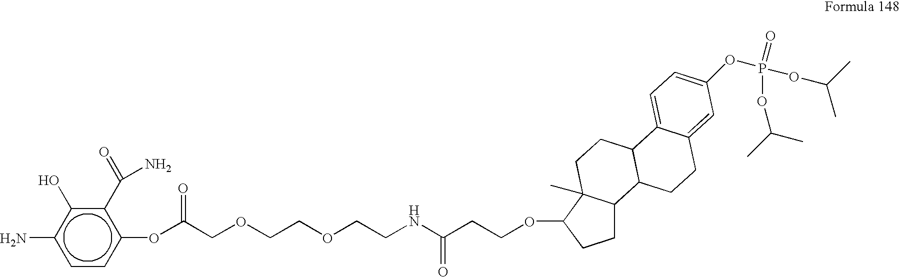

In some embodiments, the compound can be represented by the following formula, where R_{P} is derived from di-tert-butyl phosphate:

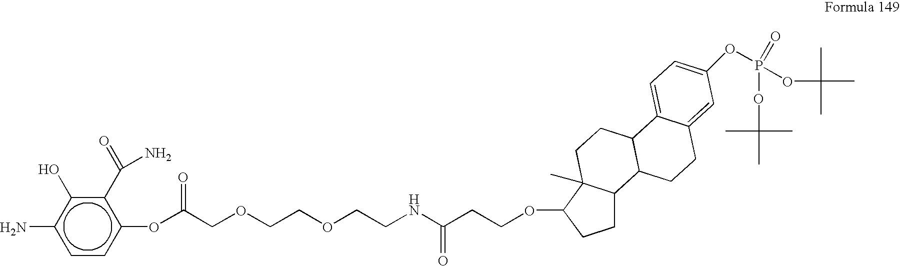

In some embodiments, the compound can be represented by the following formula, where R_{P} is derived from di-2-ethylhexyl phosphate:

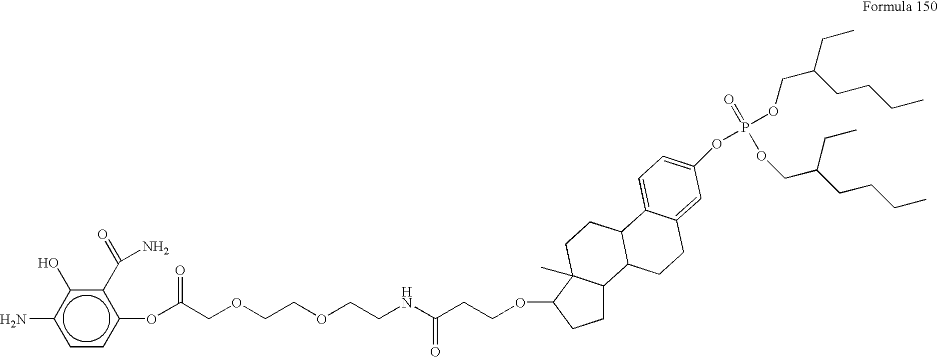

In some embodiments, the compound can be represented by the following formula, where R_{P} is derived from didodecyl phosphate:

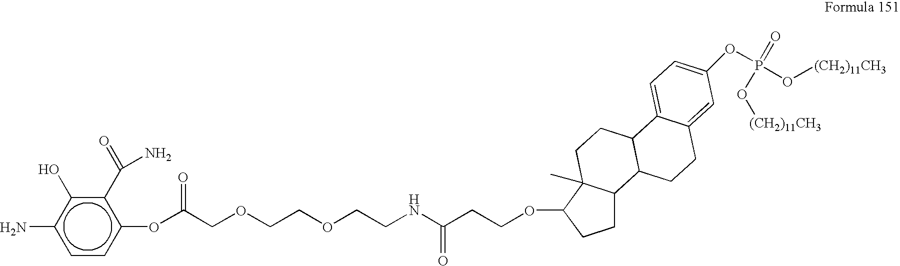

As noted above, the Bone Active Portion can be derived from an estrogenic agent. Although examples of compounds, such as the examples set forth in Formulas 142-151, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

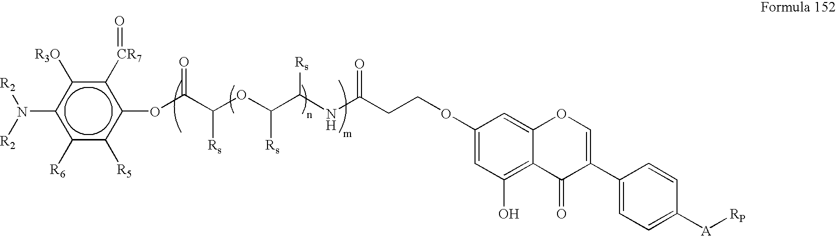

The Linking Portion can be described with reference to the following formula:

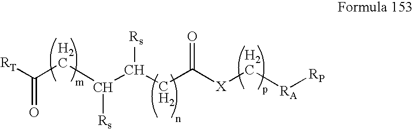

where the Linking Portion extends between R_{T} and R_{A}, where m can be 0 to about 3, n can be 0 to about 3, and p can be 0 to about 4, where m, n, and p can vary independently of one another. For example, in an exemplary embodiment, m can be 1, n can be 2, and p can be 0, as represented by the following formulas:

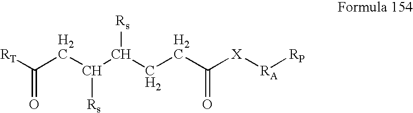

In another exemplary embodiment, m can be 3, n can be 3, and p can be 1, as be represented by the following formulas:

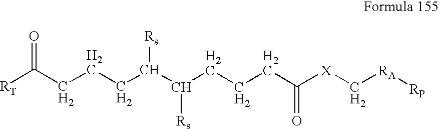

In another exemplary embodiment, m can be 0, n can be 0, and p can be 0, as be represented by the following formulas:

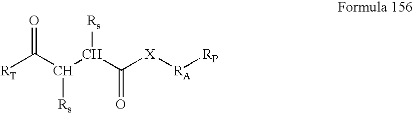

The groups of the linking portion identified as R_{S} can be hydrogen or hydroxy, and can vary independently of one another. For example, every R_{S} group can be hydroxy, as shown in the following formula, where m, n, and p are each 0:

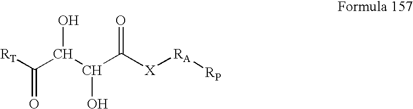

The group of the linking portion identified as X can be O, NH, S, or a covalent bond. For example, when X is O, one R_{S} group is hydrogen, the other R_{S} group is hydroxy, and m, n, and p are each 0, then an exemplary compound can be represented by the following formula:

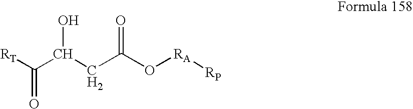

For another example, when X is NH, both R_{S} groups are hydroxy, m is 2, n is 2, and p is 2, then an exemplary compound can be represented by the following formula:

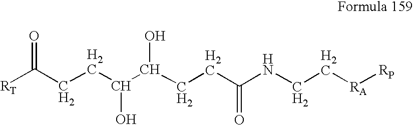

Additional exemplary embodiments of the compounds will now be described. The Linking Portion can be connected to the Bone Targeting Portion (R_{T}) in the place of R₁, R₂, or R₄. For example, when the Linking Portion is connected to the Bone Targeting Portion (R_{T}) in the place of R₁, the compound can be represented by the following formula:

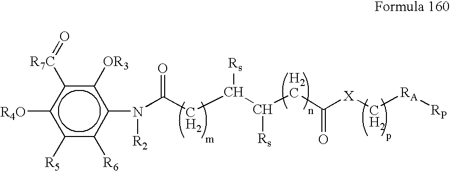

It is contemplated that the Linker Portion can be bound to the Bone Active Portion to minimize the susceptibility to hydrolysis, e.g., ether linkage, to increase the bioavailablity of the compound. That is to say, if susceptibility to hydrolysis is minimized, the compound can be delivered to and affect bone, before the Bone Active Portion can be cleaved from the compound.

In an exemplary embodiment, R_{A} can be a Bone Active Portion derived from estradiol, as represented by the following formula:

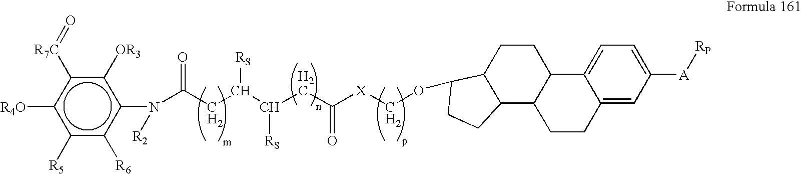

The Bone Active Portion derived from estradiol is estradiol less a hydrogen, allowing the Bone Active Portion to be connected to the remainder of the compound, while maintaining one or more activities generally associated with free estradiol, or while obtaining enhanced activity relative to estradiol.

Another exemplary compound can be represented by the following formula:

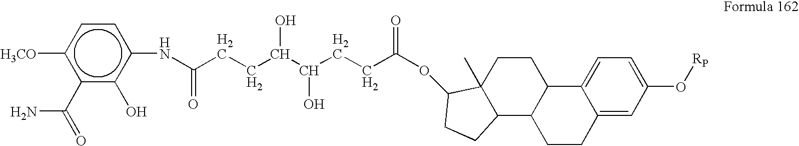

where the Linking Portion is connected to the Bone Targeting Portion (R_{T}) in the place of R₁; where R₂, R₃, R₅, and R₆ are each H, R₄ is CH₃, and R₇ is NH₂; where R₅ are each OH, m is 2, n is 2, p is 0, and X is a covalent bond; A is O; and R_{A} is derived from estradiol.

Another exemplary compound can be represented by the following formula:

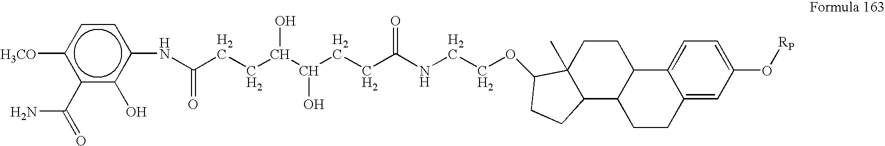

where the Linking Portion is connected to the Bone Targeting Portion (R_{T}) in the place of R₁; where R₂, R₃, R₅, and R₆ are each H, R₄ is CH₃, and R₇ is NH₂; where R₅ are each OH, m is 2, n is 2, p is 2, and X is NH; A is O; and R_{A} is derived from Estradiol.

In some embodiments, the compound can be represented by the following formula:

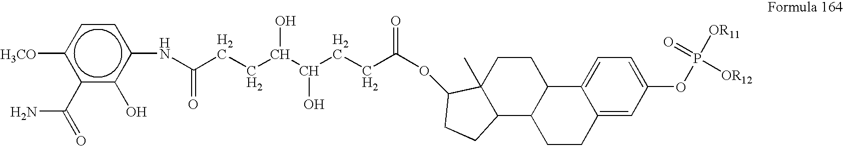

where the Linking Portion is connected to the Bone Targeting Portion (R_{T}) in the place of R₁; where R₂, R₃, R₅, and R₆ are each H, R₄ is CH₃, and R₇ is NH₂; where R₅ are each OH, m is 2, n is 2, p is 2, and X is NH; A is O; and R_{A} is derived from estradiol.

In some embodiments, the compound can be represented by the following formula, where R_{P} is derived from phosphoric acid:

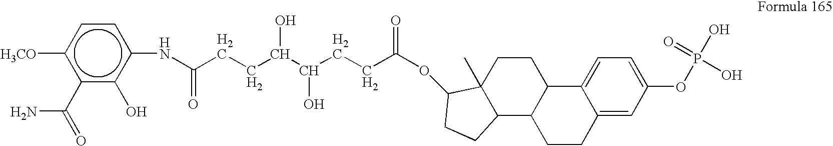

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

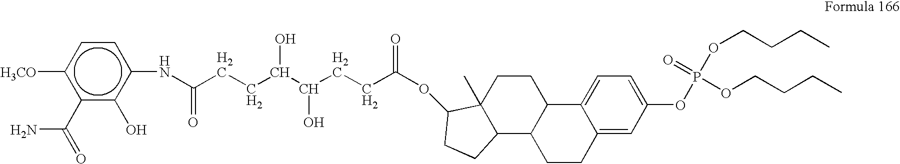

In some embodiments, the compound can be represented by the following formula, where R_{P} is derived from dibenzyl phosphate:

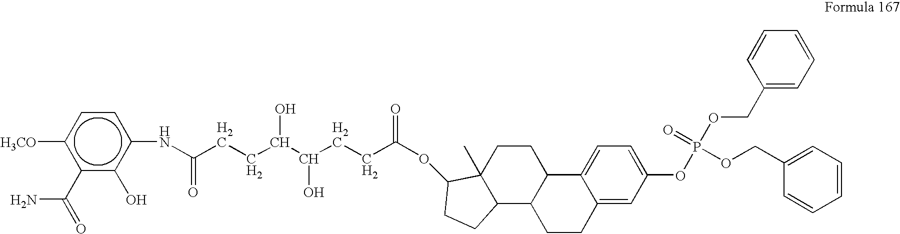

In some embodiments, the compound can be represented by the following formula, where R_{P} is derived from diisopropyl phosphate:

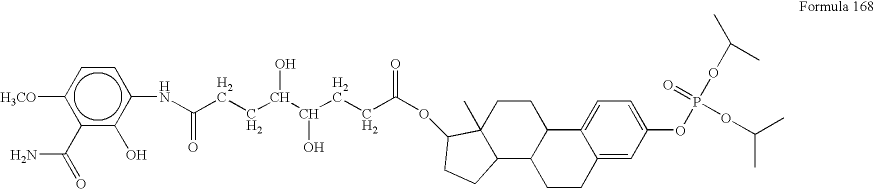

In some embodiments, the compound can be represented by the following formula, where R_{P} is derived from di-tert-butyl phosphate:

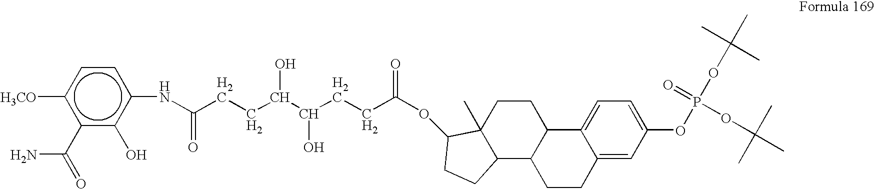

In some embodiments, the compound can be represented by the following formula, where R_{P} is derived from di-2-ethylhexyl phosphate:

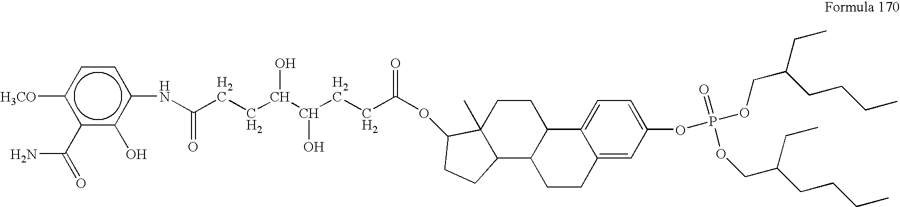

In some embodiments, the compound can be represented by the following formula, where R_{P} is derived from didodecyl phosphate:

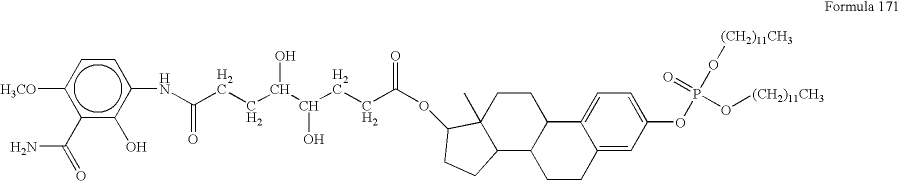

As noted above, the Bone Active Portion can be derived from an estrogenic agent. Although examples of compounds, such as the examples set forth in Formulas 161-171, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

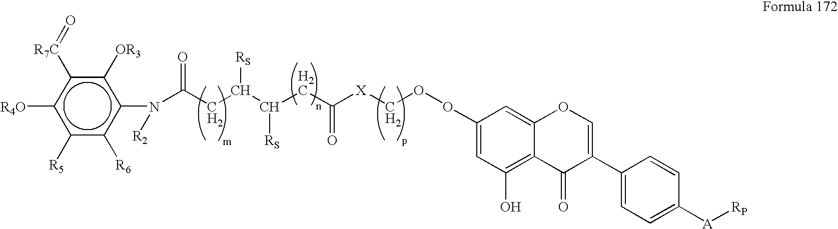

In some exemplary embodiments, the Linking Portion can be connected to the Bone Targeting Portion (R_{T}) in the place of R₄, as shown in the following formula:

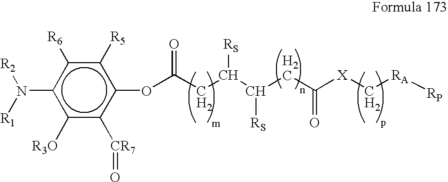

It is contemplated that the Linker Portion can be bound to the Bone Active Portion to minimize the susceptibility to hydrolysis, e.g., ether linkage, to increase the bioavailablity of the compound. That is to say, if susceptibility to hydrolysis is minimized, the compound can be delivered to and affect bone, before the Bone Active Portion can be cleaved from the compound.

In some embodiments, R_{A} can be a Bone Active Portion derived from estradiol, as represented by the following formula:

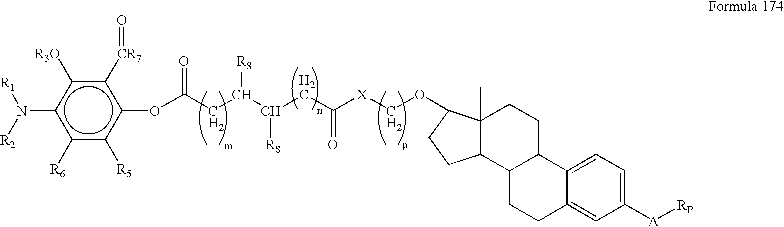

The Bone Active Portion derived from estradiol is estradiol less a hydrogen, allowing the Bone Active Portion to be connected to the remainder of the compound, while maintaining one or more activities generally associated with free estradiol, or while obtaining enhanced activity relative to estradiol.

In some embodiments, the compound can be represented by the following formula:

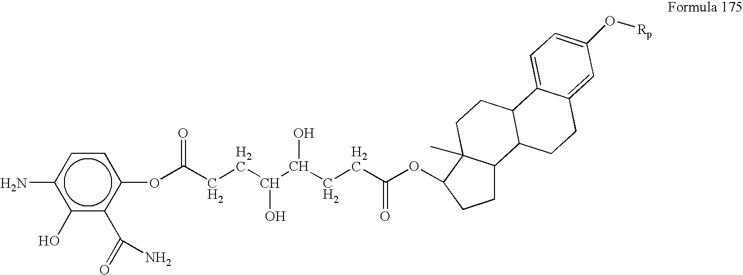

where the Linking Portion is connected to the Bone Targeting Portion (R_{T}) in the place of R₄; where R₁, R₂, R₃, R₅, and R₆ are each H, and R₇ is NH₂; where R₅ are each OH, m is 2, n is 2, p is 0, and X is a covalent bond; A is O; and where R_{A} is derived from estradiol

In some embodiments, the compound can be represented by the following formula:

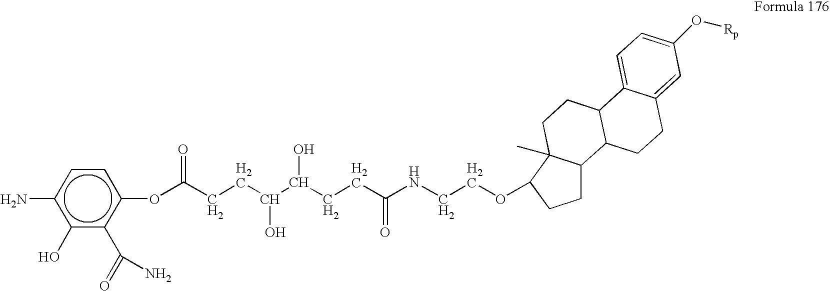

where the Linking Portion is connected to the Bone Targeting Portion (R_{T}) in the place of R₄; where R₁, R₂, R₃, R₅, and R₆ are each H, and R₇ is NH₂; where R₅ are each OH, m is 2, n is 2, p is 2, and X is NH; and where R_{A} is derived from estradiol.

In some embodiments, the compound can be represented by the following formula:

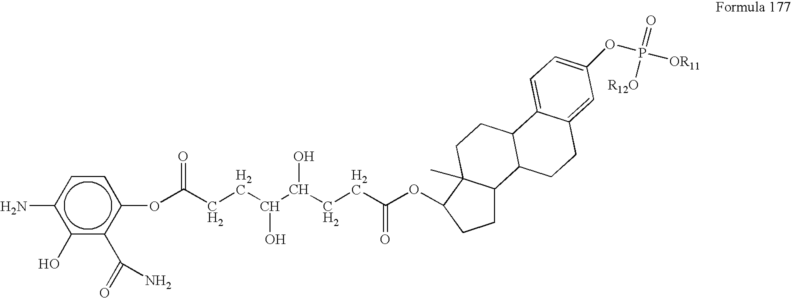

where the Linking Portion is connected to the Bone Targeting Portion (R_{T}) in the place of R₄; where R₁, R₂, R₃, R₅, and R₆ are each H, and R₇ is NH₂; where R₅ are each OH, m is 2, n is 2, p is 2, and X is NH; R_{A} is derived from Estradiol; and where R_{P} is derived from phosphoric acid.

In some embodiments, the compound can be represented by the following formula, where R_{P} is derived from phosphoric acid:

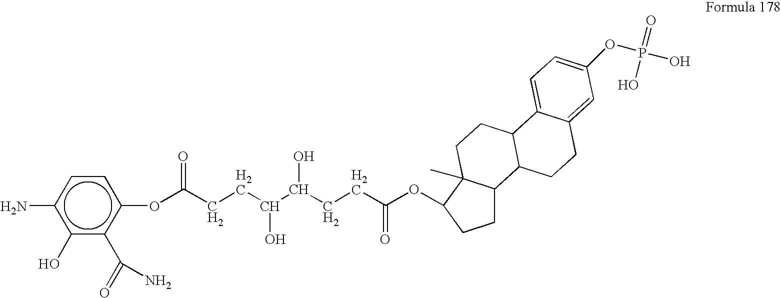

When R_{P} is derived from phosphoric acid, the compound can in some embodiments be provided as a salt, ester, or ether thereof.

In some embodiments, the compound can be represented by the following formula, where R_{P} is derived from di-n-butyl phosphate:

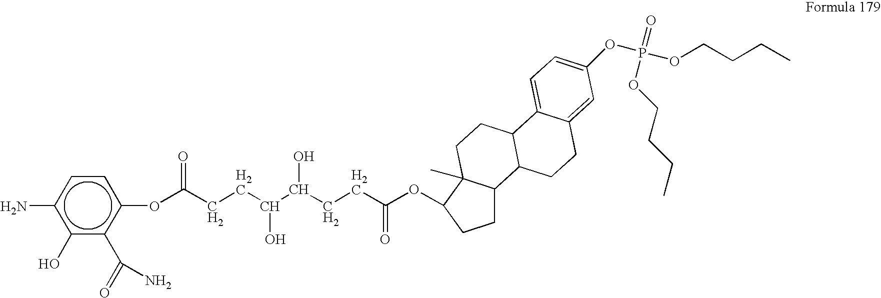

In some embodiments, the compound can be represented by the following formula, where R_{P} is derived from dibenzyl phosphate:

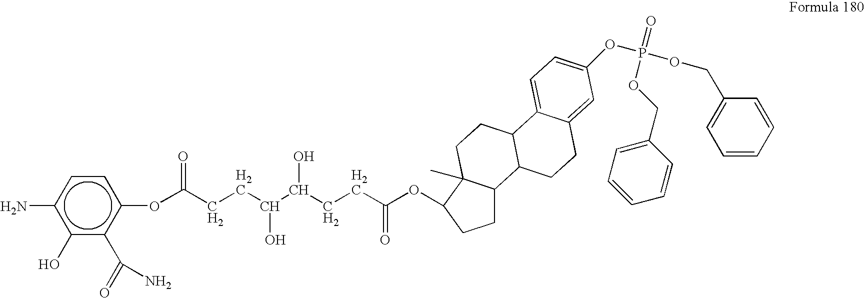

In some embodiments, the compound can be represented by the following formula, where R_{P} is derived from diisopropyl phosphate:

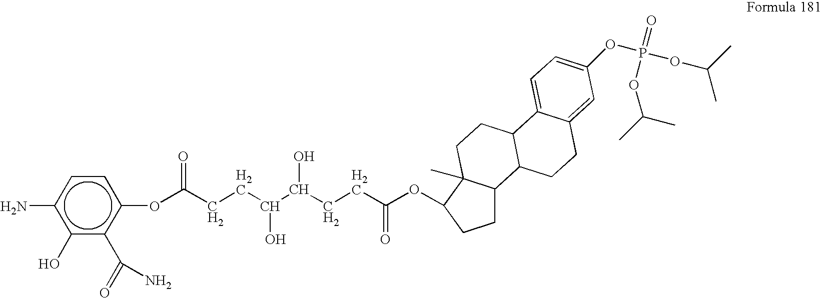

In some embodiments, the compound can be represented by the following formula, where R_{P} is derived from di-tert-butyl phosphate:

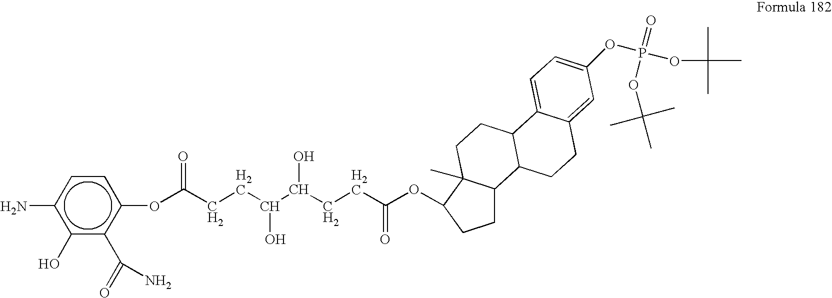

In some embodiments, the compound can be represented by the following formula, where R_{P} is derived from di-2-ethylhexyl phosphate:

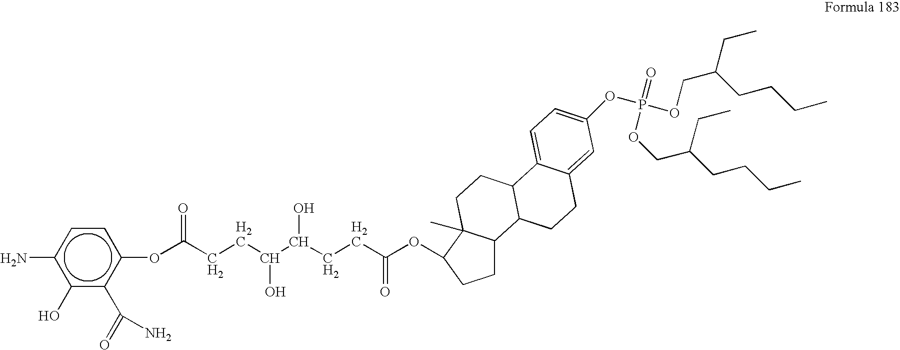

In some embodiments, the compound can be represented by the following formula, where R_{P} is derived from didodecyl phosphate:

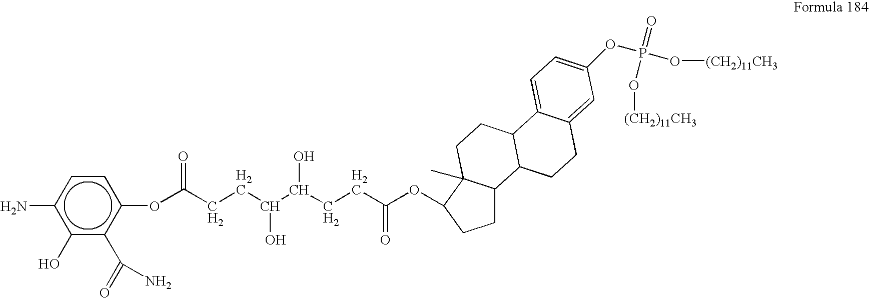

As noted above, the Bone Active Portion can be derived from an estrogenic agent. Although examples of compounds, such as the examples set forth in Formulas 174-184, include a Bone Active Portion derived from estradiol, such examples are in no way limiting. As such, it should be recognized that the presently-disclosed subject matter includes compounds including Bone Active Portions derived from other estrogenic agents, including but not limited to the estrogenic agents set forth in Table A. For example, in some embodiments, the compound can be represented by the following formula, where the Bone Active Portion is derived from genistein:

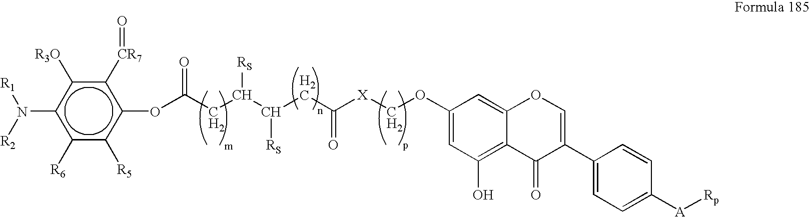

The Bone Targeting Portion (R_{T}) of the compounds of the presently-disclosed subject matter has the ability to bind to calcium with a tendency to accumulate in bone and to incorporate into its crystal lattice. The Bone Active Portion (R_{A}) of the compounds of the presently-disclosed subject matter can be derived from an estrogenic agent, and can interact with bone and affects bone metabolism.

The performance of the compounds of the presently-disclosed subject matter can be facilitated, first by the Bone Targeting Portion (R_{T}), which localizes the compound at the bone site. The Blocking Group (R_{P}) can then be cleaved from the compound by one or more enzymes present at the bone. Once anchored at the bone site with its activity restored, the Bone Active Portion (R_{A}) can interact with and affect the bone.

Without wishing to be bound by theory or mechanism, it is believed that the compounds of the presently-disclosed subject matter interact with the calcium in the bone in the following manner, which is described using an exemplary embodiment of the presently-disclosed subject matter:

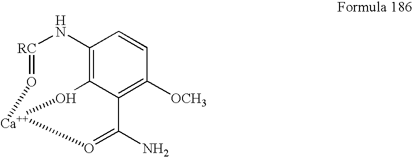

As shown by the example, in some embodiments, three positions of the Bone Targeting Portion (R_{T}) can interact with calcium, facilitating the localization of the compound to bone. In the exemplary compound of Formula 186, the R₄ group (CH₃) is not depicted as interacting with the calcium; however, it is contemplated that the R₄ group can affect the affinity for bone. Without wishing to be bound by theory or mechanism, it is believed that the affinity for bone can be modulated, in part, by strategically selecting the R₄ group based on its electron-donating properties. The greater the electron donating properties of the R₄ group, the greater the affinity for bone, i.e., a negative charge is directed from the side of the Bone Targeting Portion having the R₄ group, towards the side of the Bone Targeting Portion thought to interact with the calcium, thereby creating a stronger interaction between the Bone Targeting Portion and the positively-charged calcium.

Some embodiments of compounds of the presently-disclosed subject matter are described with reference to formulas. Some formulas include portions depicting a particular stereoisomer of one or more moieties of the compound. Such depicted stereoisomers are representative of some embodiments of the compounds; however, the formulas are intended to encompass all active stereoisomers of the depicted compounds.

The compounds of the presently-disclosed subject matter can in some embodiments contain one or more asymmetric carbon atoms and can exist in racemic and optically active forms. Depending upon the substituents, the present compounds can form addition salts as well. All of these other forms are contemplated to be within the scope of the presently-disclosed subject matter. The compounds of the presently-disclosed subject matter can exist in stereoisomeric forms and the products obtained thus can be mixtures of the isomers.

The presently-disclosed subject matter includes methods for treating bone conditions in a subject. Methods include administering to the subject an effective amount of a compound of the presently-disclosed subject matter, as described above.

As used herein, the terms treatment or treating relate to any treatment of a bone condition of interest, including but not limited to prophylactic treatment and therapeutic treatment As such, the terms treatment or treating include, but are not limited to: preventing a condition of interest or the development of a condition of interest; inhibiting the progression of a condition of interest; arresting or preventing the development of a bone condition of interest; reducing the severity of a condition of interest; ameliorating or relieving symptoms associated with a condition of interest; and causing a regression of the condition of interest or one or more of the symptoms associated with the condition of interest. Examples of conditions of interest are noted herein. For example, in some embodiments, the condition of interest can be a primary or secondary bone condition of interest.

As noted above, in some embodiments, the bone condition of interest is a metabolic bone disease (MBD), wherein treatment can result in an anti-catabolic effect and/or an anabolic effect. In some embodiments, the bone condition of interest is a bone fracture, wherein treatment can result in an anabolic effect. Other conditions of interest and/or desired effects are noted herein and/or are contemplated by the presently-disclosed subject matter.

As used herein, the term effective amount refers to a dosage sufficient to provide treatment for the bone condition of interest being treated. This can vary depending on the patient, the condition, and the treatment being effected. The exact amount that is required will vary from subject to subject, depending on the species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation.

As noted above, in some embodiments, the compound can be provided as a pharmaceutically-acceptable salt or solvate. Suitable acids and/suitable bases, as will be known to those of ordinary skill in the art, are capable of forming salts of the compounds described herein, e.g., hydrochloric acid (HCl), sodium hydroxide. A solvate is a complex or aggregate formed by one or more molecules of a solute, e.g. a compound or a pharmaceutically-acceptable salt thereof, and one or more molecules of a solvent. Such solvates can be crystalline solids having a substantially fixed molar ratio of solute and solvent. Suitable solvents will be known by those of ordinary skill in the art, e.g., water, ethanol.

As will be understood by those of ordinary skill in the art, a dosage regimen can be adjusted to provide an optimum treatment effect and can be administered daily, biweekly, weekly, bimonthly, monthly, or at other appropriate time intervals. As will be understood by those of ordinary skill in the art, compounds of the presently-disclosed subject matter can be administered orally, intravenously, intramuscularly, subcutaneously, or by other art-recognized means.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art. Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in a conventional manner.

The compounds can also be formulated as a preparation for injection. Thus, for example, the compounds can be formulated with a suitable carrier. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

As used herein, the term subject refers to humans and other animals. Thus, veterinary uses are provided in accordance with the presently disclosed subject matter. The presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter is further illustrated by the following specific but non-limiting examples. The following examples may include compilations of data that are representative of data gathered at various times during the course of development and experimentation related to the presently-disclosed subject matter. The following examples include some examples that are prophetic.

The presently-disclosed subject matter is further illustrated by the following specific but non-limiting examples. The following examples may include compilations of data that are representative of data gathered at various times during the course of development and experimentation related to the presently-disclosed subject matter. The following examples include some examples and statements that are prophetic.

### EXAMPLES

Syntheses. The compounds of the presently-disclosed subject matter can be prepared in accordance with the exemplary schemes set forth in the Examples herein, and by techniques known to those of ordinary skill in the art.

### Exemplary Synthesis where R_{A} is Derived from Estradiol

The following is exemplary where R_{A} is a Bone Active Portion derived from estradiol.

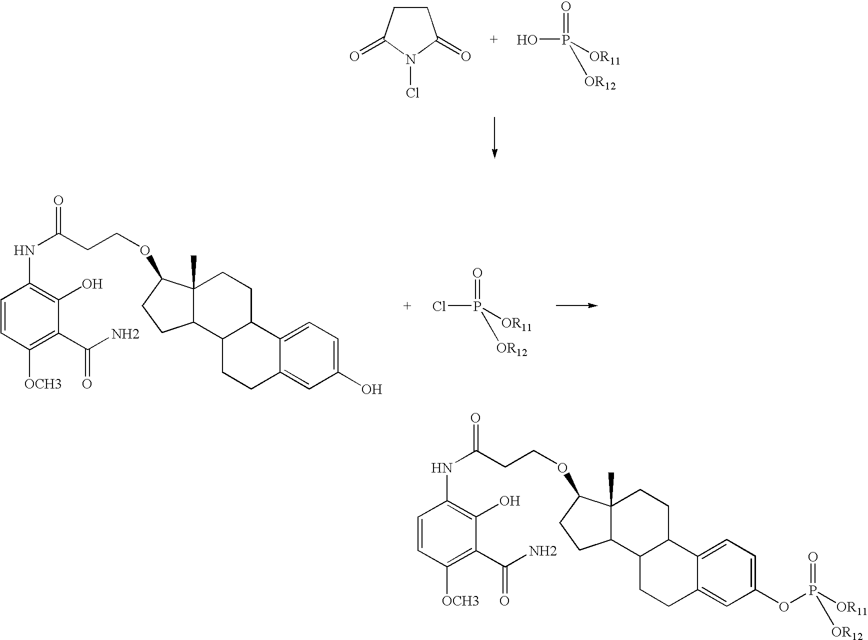

Dialkyl or dibenzyl phosphites used for the synthesis can be obtained from commercial sources, for example, Sigma-Aldrich Chemical Co., St. Louis, Mo., U.S.A. For example, phosphoric acid can be used to prepare the compound of Formula 65; di-n-butyl phosphate can be used to prepare the compound of Formula 66, dibenzyl phosphate can be used to prepare the compound of formula 67, diisopropyl phosphate can be used to prepare the compound of Formula 68; di-tert-butyl phosphate can be used to prepare the compound of Formula 69; di-2-ethylhexyl phosphate can be used to prepare the compound of Formula 70; and didodecyl phosphate can be used to prepare the compound of Formula 71.

The desired dialkyl or dibenzyl phosphite (1.0 mmol) was added dropwise to a stirring suspension of N-chlorosuccinimide (1.20 mmol) in dry toluene (10 mL). The resulting suspension was stirred at ambient temperature for 3 h before being filtered. The filtrate was evaporated and extracted with hexane (30 mL) to remove the product from succinimide. Dialkyl or dibenzyl chlorophosphites (R₁₁═R₁₂=n-butyl, dibenzyl, isopropyl, tert-butyl, 2-ethylhexylester, or dodecylester) were obtained as colorless oils (90-98%) upon evaporation of the hexane extracts. All the products gave expected NMR and MS fragmentational data.

n-butylithium (2.5 M solution in hexane), 0.8 mL, (diluted 10 times before addition) was then slowly added to a solution of coupled product. (0.1 g, 0.196 mmol) in dry THF (3.0 mL) at 0° C. under argon, which resulted in formation of a white precipitate The resulting suspension was stirred for 5 min. and then the dialkyl phosphoryl chloride (1.0 equiv) was added to the reaction mixture. The suspension, which cleared after 5-10 min, was stirred at room temperature for 18 h before being evaporated to dryness. Four spots were observed on TLC in CHCl₃: MeOH (3%). The crude product was purified by preparative TLC silica gel plates eluting with CHCl₃: MeOH 3%. White solid to colorless oil products were obtained (10-18 mg, 14-16% yield). Rf=0.45 (97:3; CHCl3/MeOH).

The following is another example of a synthesis scheme for preparing the compound of Formula 66.

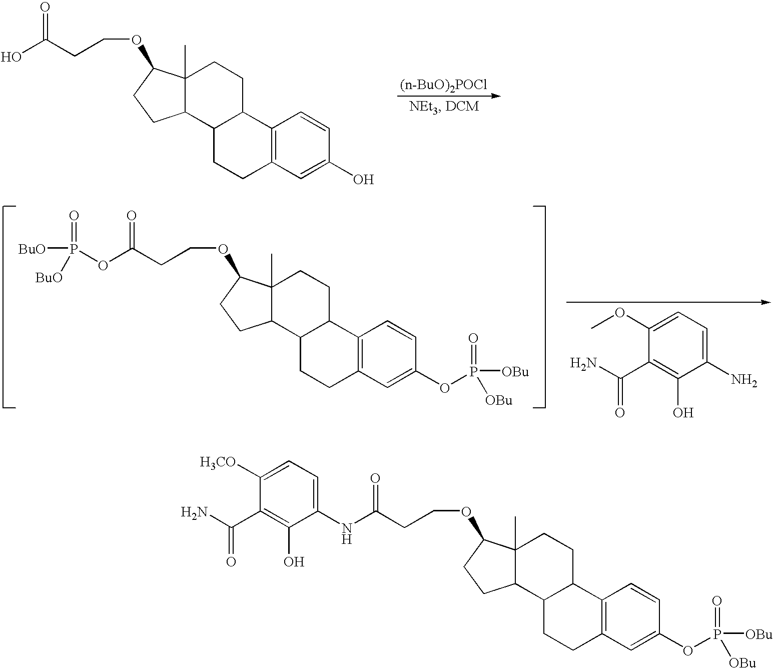

The following is exemplary where R_{A} is a Bone Active Portion derived from estradiol.

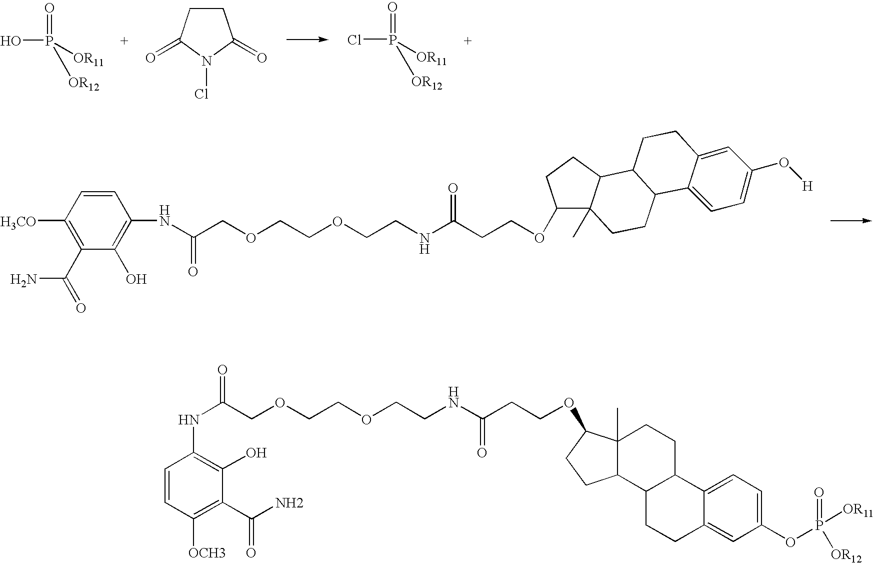

Dialkyl or dibenzyl phosphites used for the synthesis can be obtained from commercial sources, for example, Sigma-Aldrich Chemical Co., St. Louis, Mo., U.S.A. For example, phosphoric acid can be used to prepare the compound of Formula 133; di-n-butyl phosphate can be used to prepare the compound of Formula 134, dibenzyl phosphate can be used to prepare the compound of formula 135, diisopropyl phosphate can be used to prepare the compound of Formula 136; di-tert-butyl phosphate can be used to prepare the compound of Formula 137; di-2-ethylhexyl phosphate can be used to prepare the compound of Formula 138; and didodecyl phosphate can be used to prepare the compound of Formula 139.

### Exemplary Synthesis where R_{A} is Derived from Genistein

The following is exemplary where R_{A} is a Bone Active Portion derived from genistein.

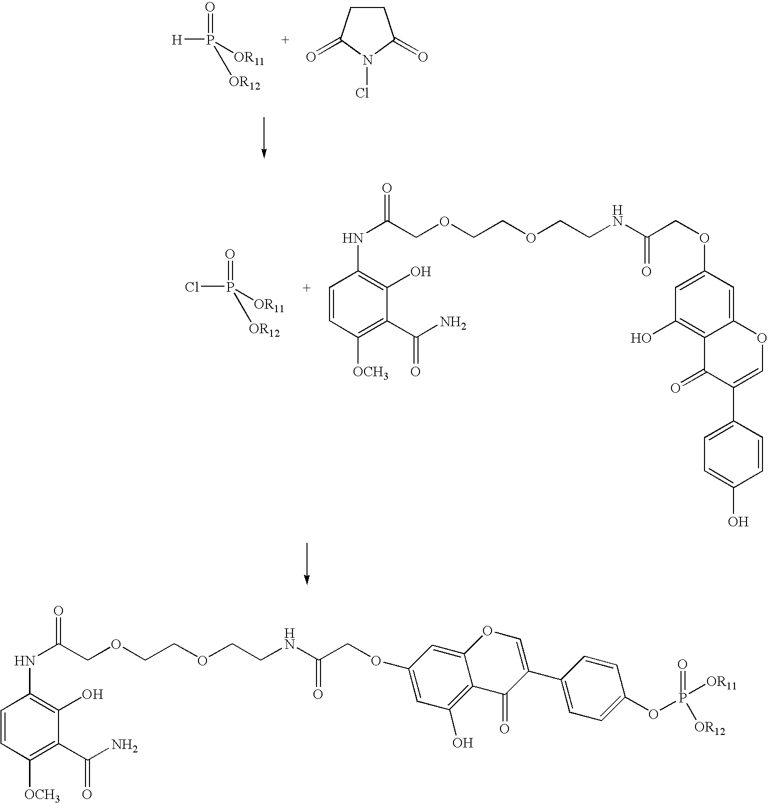

Dialkyl or dibenzyl phosphites used for the synthesis can be obtained from commercial sources, for example, Sigma-Aldrich Chemical Co., St. Louis, Mo., U.S.A. For example, phosphoric acid, di-n-butyl phosphate, dibenzyl phosphate, diisopropyl phosphate, di-tert-butyl phosphate, di-2-ethylhexyl phosphate, or didodecyl phosphate could be selected.

Other compounds of the presently-disclosed subject matter can be prepared in manners similar to those described above, as will be apparent to those of ordinary skill in the art upon review of this disclosure. Additional information relevant to the synthesis of compounds can also be found in U.S. patent application Ser. No. 12/036,057 to Pierce, et al., entitled, “Methods and Compounds for the Targeted Delivery of Agents to Bone for Interaction Therewith,” which is incorporated herein in its entirety by this reference.

### Targeting Bone

A Hydroxyapatite (HA) Binding Assay is used to determine whether compounds have an affinity for bone. Compounds of the presently-disclosed subject matter having a Bone Targeting Portion (R_{T}) are studied using the HA Binding Assay. A 10⁻³ M solution of each analyte was made in 100% dimethylsulfoxide (DMSO). A 100 fold dilution was then made to form a 10⁻⁵ M solution in 50 mM Tris-HCl buffer, pH 7.4, 1% DMSO. Tetracycline was used as a reference analyte and approximately 50% was adsorbed to HA at the concentration of 10⁻⁵ M. The HA slurry was 0.5 g/100 mL 50 mM Tris-HCl buffer, 1% DMSO.

For each analyte, two samples were prepared. For one sample, 1 mL of 10⁻⁵ M analyte and 100 μL 50 mM Tris-HCl buffer, 1% DMSO was pipetted into a microcentrifuge tube. For the second sample, 1 mL of 10⁻⁵ M analyte and 100 μL of the HA slurry was pipetted into a microcentrifuge tube. The samples were mixed gently by inversion for 4 minutes and then centrifuged at 12,000 g for 3 minutes to sediment the HA contained in those samples. The supernatant was transferred to another microcentrifuge tube.

An electronic spectral scan (ultraviolet-visible) from 220-520 nm was obtained for each analyte using a Varian Cary 300 Bio Scan. The blank was 50 mM Tris-HCl buffer, 1% DMSO. The wavelength of maximum absorbance (λₘₐₓ) was determined, and the extinction coefficient (ε) was calculated using the Beer-Lambert Law.

The absorbance of the samples incubated with HA was measured at λₘₐₓ, and the molar concentration of the analyte was then determined using the Beer-Lambert Law and the previously calculated extinction coefficient. The fraction adsorbed to HA for each sample was subsequently calculated.

Binding to Hydroxyapatite

| Compound | Binding Index |
|---|---|
| 17,β-estradiol | −8 |
| tetracycline | 100 |
| BTE2-D2-3-O-di-n-butyl ester | 100 |
| (compound of Formula 66) | |
| BTE2-D2-3-O-dibenzyl ester | 190 |
| (compound of Formula 67) | |
| BTE2-D2-3-O-diisopropyl ester | 200 |
| (compound of Formula 68) | |
| BTE2-D2-3-O-di-tert-butylester | 180 |
| (compound of Formula 69) | |
| BTE2-D2-3-O-di-2-ethylhexylester | 180 |
| (compound of Formula 70) | |
| BTE2-D2-3-O-didodecylester | 160 |
| (compound of Formula 71) | |

Other compounds of the presently-disclosed subject matter, including the compounds of Formulas 65 and 133-139, compounds including a Bone Active Portion derived from genistein, and compounds including a Bone Active Portion derived from other estrogenic agents were tested and determined to have an affinity for bone.

### Affecting Bone

Animals. Six month old, bilaterally ovariectomized (OVX) or sham operated Sprague-Dawley female rats (Harlan Laboratories, Indianapolis, Ind.) were maintained at the University of Louisville Research Resources Center at 22° C. with a 12-h light/dark cycle and ad libitum access to tap water and rodent chow (Purina Laboratory Rodent Diet 5001). All animal procedures were approved by the Institutional Animal Care and Use Committee, which is certified by the American Association for Accreditation of Laboratory Animal Care.

In vivo experiments were conducted in order to investigate the efficacy of compounds of the presently-disclosed subject matter. In all of the experiments, OVX and sham operated rats were randomly divided into groups of 5-7 animals. In all of the studies experimental groups included: i) Sham-operated control (euthanized six weeks post surgery as a pretreatment control), ii) OVX control (euthanized six weeks post surgery as a pretreatment control), iii) Sham control receiving vehicle, iv) OVX control receiving vehicle, v) OVX receiving 17-ethinyl estradiol (equimolar concentration with a selected test compound), vi) OVX receiving alendronate (1.6 mg/kg), and vii) OVX receiving parathyroid hormone 1-34 (PTH) (80 μg/kg). 17-ethinyl estradiol (17 EE) is a free estrogenic agent known to be orally active, and can serve as an example of an anti-catabolic agent. Alendronate (Alen) is a bisphosphonate that is currently used to treat osteoporosis (e.g., Fosamax®, Merck & Co., Inc,), and can serve as an example of an anti-catabolic agent. Parathyroid hormone 1-34 (PTH) is an agent that can serve as an example of an anabolic agent. All compounds and vehicle (1% DMSO in corn oil) were administered three times per week orally by gavage except for PTH which was administered via a subcutaneous injection in a volume of 0.5 ml/kg body mass thrice per week. Compound administration was initiated 6 weeks following surgery and lasted for 6 weeks (18 doses total).

Compounds of the presently-disclosed subject matter were orally administered to OVX rats at various doses. As indicated above all BTE's were administered three times per week for 6 weeks.

Following 6 weeks of treatment, blood was obtained from each animal via cardiac puncture following an overnight fast and animals were subsequently euthanized via carbon dioxide asphyxiation. Blood was centrifuged immediately and the obtained serum samples were aliquoted and stored at −70° C. prior to analysis. Uteri were removed and fresh weights were obtained. Uterine masses were normalized to body mass at the end of the experiment. The left and right femora and left and right tibiae were subsequently collected from each animal, cleaned of soft tissue, and stored in saline at 4° C. prior to analysis.

Estrogenic Effect.

Quantitative Determination of Lipid Metabolism (Total Cholesterol HDL, and LDL) in Rat Serum. Total cholesterol, high-density lipoprotein (HDL), and low-density lipoprotein (LDL) were quantitatively determined in the serum of rats in order to evaluate the extraosseous effects of estrogen treatment on lipid metabolism. Commercially available kits from Wako Diagnostics (Richmond, Va.) were used for the evaluations as recommended by the manufacturer.

Uterine Mass and Body Weight. Ovariectomized (OVX) animals generally exhibit an increase in body weight; however, upon treatment with free estrogen, animals generally exhibit a decrease in body weight to a normal body weight. As such, an assessment of whole body estrogenic effect exhibited in the animals can be made by measuring body weight of the animals following treatment. Following OVX, animals exhibit a decrease in uterine mass, and a subsequent increase in uterine mass upon treatment with free estrogen. Uterine mass can be used to as a measure of estrogenic effect occurring in a tissue outside the bone in response to treatment with a test compound or composition. Generally, a lower uterine mass can be associated with a decreased risk of adverse side effects associated with the test compound or composition. A ratio of uterine mass to body weight can be used to correct for any increase in uterine size attributable to the relative size of the individual animal. Body weight and/or uterine mass also serve as an indirect measures of toxicity of a test compound.

Bone Density Effects.

Femoral Density via Archimedes' Principle. Right femora were submerged in distilled water and fully hydrated under a vacuum for 1 hr. Subsequently, the mass of each hydrated femur was obtained in air and when submerged in water. Densities were determined using Archimedes' Principle according to the following formula: density=[mass of hydrated femur/(mass of hydrated femur−mass of hydrated femur submerged in water)]×density of distilled water at a given temperature (See Keenan, et al. Comparison of bone density measurement techniques: DXA and Archimedes' principle. J Bone Miner Res 1997; 12:1903-7.). The obtained density measurements were associated with whole bone.

Regional Femoral Density via Archimedes' Principle. In addition to whole bone measurements, assessments of the densities at different regions of the bone were made. The proximal and distal ends of the femur are comprised primarily of cancellous/trabecular bone, whereas the femoral diaphysis contains primarily cortical bone. It is in these regions of bone that problems, for example, in osteoporosis typically occur. Evaluation of the density of each of these femoral regions can thereby increase the understanding of the effect of a compound on each of these types of bone. The left femora were separated into three regions (proximal left femur, distal left femur, and left femoral diaphysis) using an Isomet Low Speed Precision Sectioning Saw from Buehler Limited (Lake Bluff, Ill.) with a diamond blade. Briefly, each femur was measured with a Cen-Tech Digital Caliper and a cut was made from each end at 20% of the length of the femur plus half the width of the blade. Subsequently, the bone marrow was washed out of the femoral diaphysis and the Archimedes density for each of the three femoral regions was determined as described above.

Volume Fraction—Ex vivo micro-computed tomography (μCT). Volume fraction is a representative value for the amount of bone that occupies a given volume or space. High resolution image data were collected using a customized micro-CT system (ACTIS 150/225 system, BIR Inc., Lincolnshire, Ill.). The metaphysis of each right tibia was scanned over a three millimeter range and three-dimensional images were reconstructed. Data were subsequently processed to reveal the volume fraction (BV/TV) occupied by trabecular (cancellous) bone tissue, cortical bone tissue, and whole bone tissue. The resulting data provides information regarding the density of whole bone, cortical bone, and trabecular bone.

Bone Strength.

Bone Mechanical Competence Indentation Test. After sacrifice, the left tibiae were trimmed to expose the cancellous bone of the proximal tibial metaphysis and an indentation test was performed by advancing a flat-tipped cylindrical post (1.5 mm diameter) axially into the cut surface to measure the compressive strength of the cancellous bone structure.

Bone Formation and Turnover.

Rat Osteocalcin EIA. Osteocalcin is a hydroxyapatite-binding protein that is synthesized by osteoblasts during bone formation. Thereby, serum osteocalcin levels are commonly used as a biochemical marker of bone formation. Rat serum osteocalcin levels were measured using the Rat Osteocalcin EIA Kit from Biomedical Technologies, Incorporated (Stoughton, Mass.) as recommended by the manufacturer.

RatLaps™ ELISA for C-telopeptide Fragments of Collagen Type I (CTX-I). Osteoclast mediated breakdown of collagen type I in bone leads to the release of free and peptide bound fragments of the collagen type I molecule. The fragment released from the carboxy-terminal region of collagen type I is termed the C-telopeptide fragment of collagen type I (CTX-I) and is commonly used as a biochemical marker of bone resorption. Bone resorption (CTX-I) was quantitatively assessed in rat serum using the commercially available RatLaps™ ELISA KIT from Nordic Bioscience Diagnostics A/S (Herlev, Denmark) as recommended by the manufacturer.

Stimulation of Periosteal Bone Formation in a Mouse Calvarial Injection Model. The anabolic (bone formation) potential of compounds of the presently-disclosed subject matter are evaluated in the mouse calvarial injection model. Briefly, 4-week-old ICR Swiss mice are injected subcutaneously over the surface of the calvariae with compounds of the presently-disclosed subject matter at concentrations of 0, 1, 3, and 10 mg/kg/day for 5 days (twice a day). Microtubule inhibitor TN-16 (5 mg/kg/day for 2 days, twice a day) are used as a positive control. Mice are sacrificed two weeks after the injections are completed. Dissected calvarial samples are fixed in 10% phosphate-buffered formalin for 2 days, decalcified in 10% EDTA for 2 weeks and then embedded in paraffin. Histological sections are cut and stained with H&E and orange G. New woven bone formation (new bone area) is quantified by histomorphometry using the OsteoMeasure system (OsteoMetrics Inc., Atlanta, Ga.).

Bone Targeting Portion (R_{T})

Animals were treated with the following compounds having an affinity for bone as assessed by HA-binding assay, including bone targeting portions (R_{T}), but lacking linking portions (R_{L}) and bone active portions (R_{A}). Samples were collected and studied as described above.

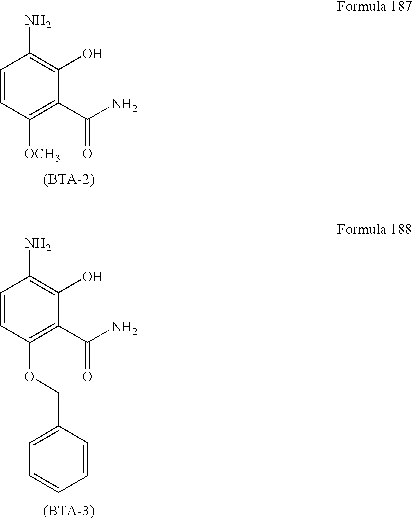

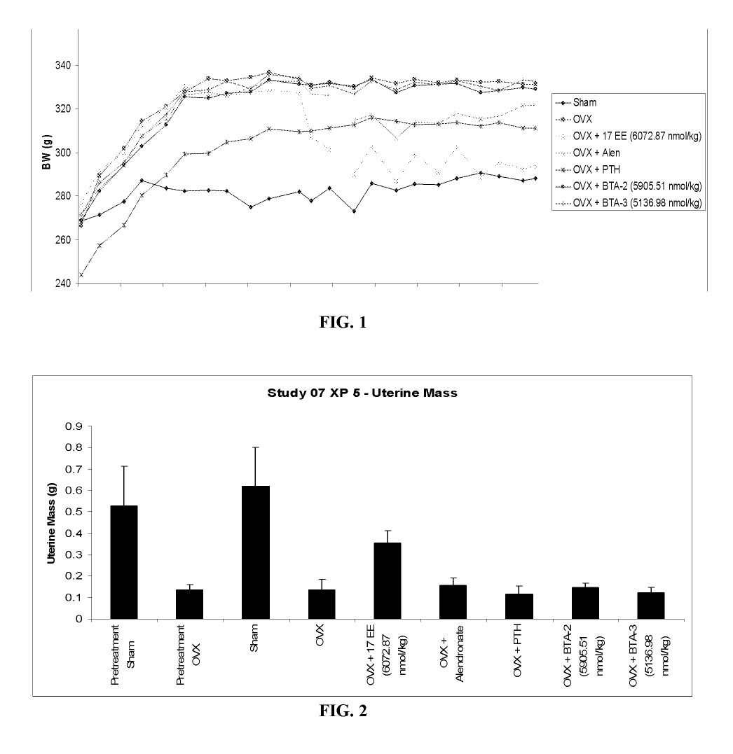

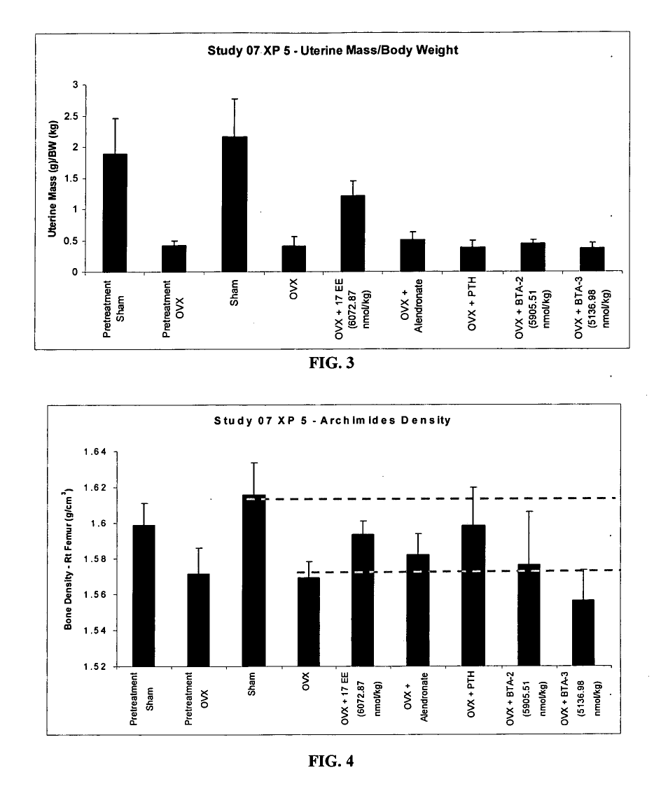

With reference to FIG. 1, the body weights of the animals were measured at regular time intervals and body weights were plotted as a function of time. Body weight can serve as an indirect measure of toxicity. The compounds of Formulas 187 (BTA-2) and 188 (BTA-3) are shown to have no effect on body weight. Turning now to FIGS. 2 and 3 the uterine mass of each animal was measured and expressed both independently, and as a ratio of uterine mass to body weight. The compounds of Formulas 187 (BTA-2) and 188 (BTA-3) are shown to have no effect on uterine mass, nor ration of uterine mass to body weight.

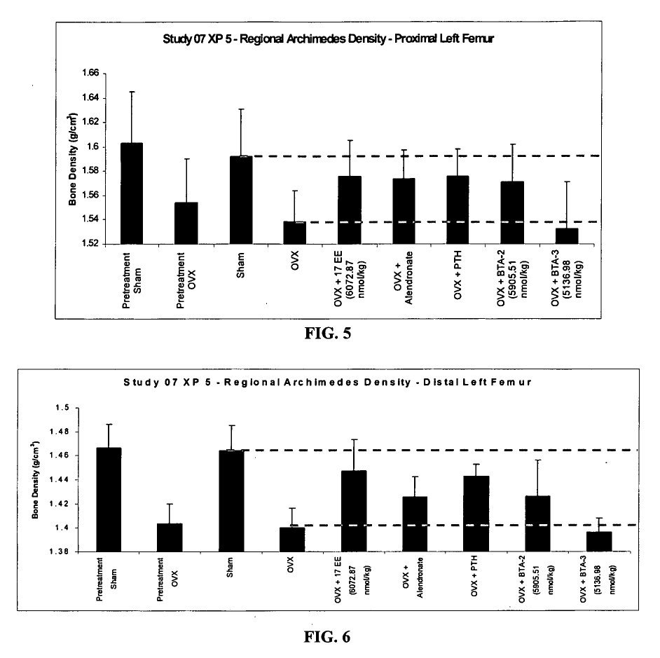

The right femora were used to assess bone density, as described above. With reference to FIG. 4, whole bone density was not significantly effected by compounds of Formulas 187 (BTA-2) or 188 (BTA-3). Regional bone density was also assessed, as described above. With reference to FIGS. 5 and 6, regional bone density was not significantly effected by compounds of Formulas 187 (BTA-2) or 188 (BTA-3). Compound including Bone Active Portion (R_{A}) derived from Estradiol.

Animals were treated with compounds including a bone active portion (R_{A}) derived from estradiol. The compounds of Formulas 66 (BTE2-D2-3-O-di-n-butyl ester) and 67 (BTE2-D2-3-O-dibenzyl ester) were orally administered to animals at doses of 25 or 250 μg/kg.

Body Weight and Uterine Mass.

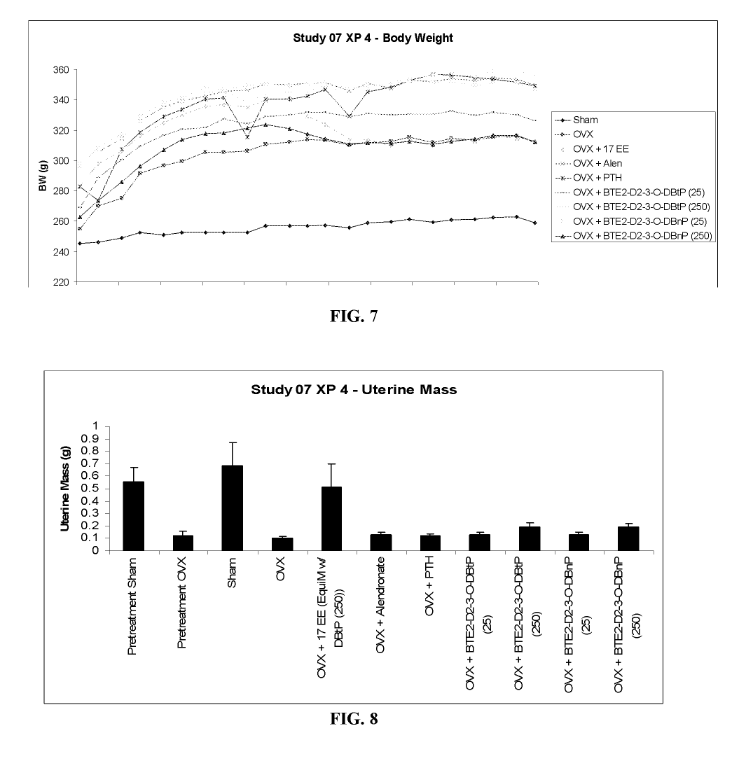

With reference to FIG. 7, the body weights of the animals were measured at regular time intervals and body weights were plotted as a function of time. Body weight can serve as an indirect measure of toxicity. The compound of Formulas 66 (BTE2-D2-3-O-di-n-butyl ester) and 67 (BTE2-D2-3-O-dibenzyl ester) had limited or no effect on body weight.

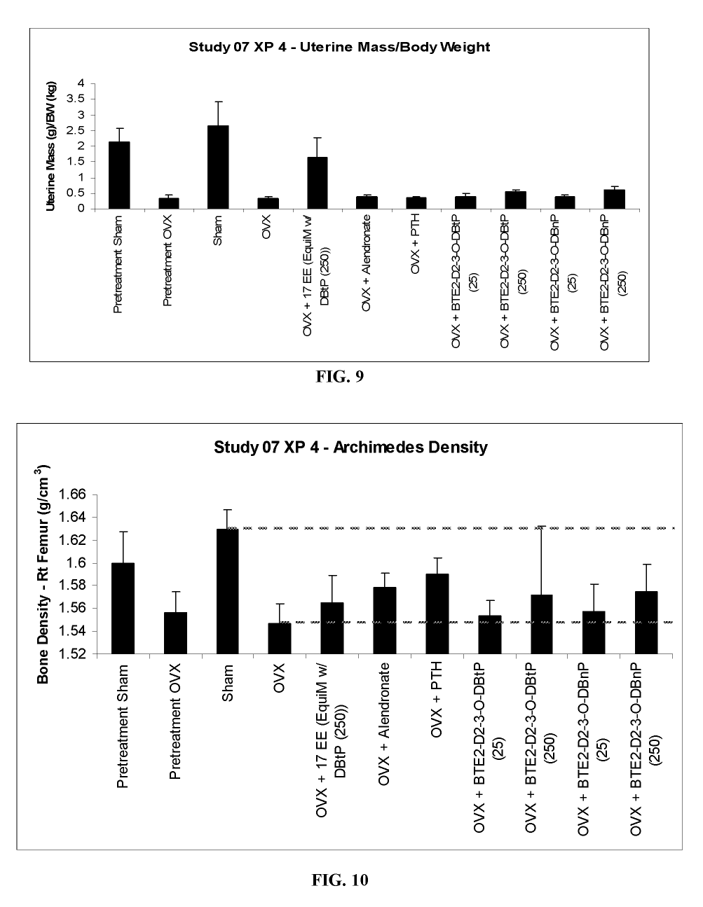

Turning now to FIGS. 8 and 9 the uterine mass of each animal was measured and expressed both independently, and as a ratio of uterine mass to body weight ratio. Animals treated with the compounds of Formulas 66 (BTE2-D2-3-O-di-n-butyl ester) and 67 (BTE2-D2-3-O-dibenzyl ester) had limited or no effect on uterine masses or uterine mass to body weight ratio, whereas the free steroidal estrogenic agent, 17-ethinyl estradiol, induced an increase in uterine mass and uterine mass to body weight ratio. This is a surprising result given that the compounds of Formulas 66 and 67 include a bone active portion derived from the steroidal estrogenic agent, estradiol. These results indicate that the compounds of the presently-disclosed subject matter including a bone active portion derived from a estrogenic agent do not act in the same manner as free estrogenic agents.

Lipid Metabolism.

Total cholesterol, high-density lipoprotein (HDL), and low-density lipoprotein (LDL) in collected serum samples are quantitatively determined. The compounds of Formulas 66 (BTE2-D2-3-O-di-n-butyl ester) and 67 (BTE2-D2-3-O-dibenzyl ester) have a limited or no effect on lipid metabolism.

Bone Density Effects.

Whole Bone Density.

The right femora were used to assess bone density, as described above. With reference to FIG. 10, whole bone density was affected by the compounds of Formulas 66 (BTE2-D2-3-O-di-n-butyl ester) and 67 (BTE2-D2-3-O-dibenzyl ester). Animals receiving the compounds of Formulas 66 and 67 were shown to have whole bone densities in the same ranges as those animals receiving 17-ethinyl estradiol (17 EE), Alendronate (Alen), or Parathyroid hormone 1-34 (PTH).

Regional Femoral Density.

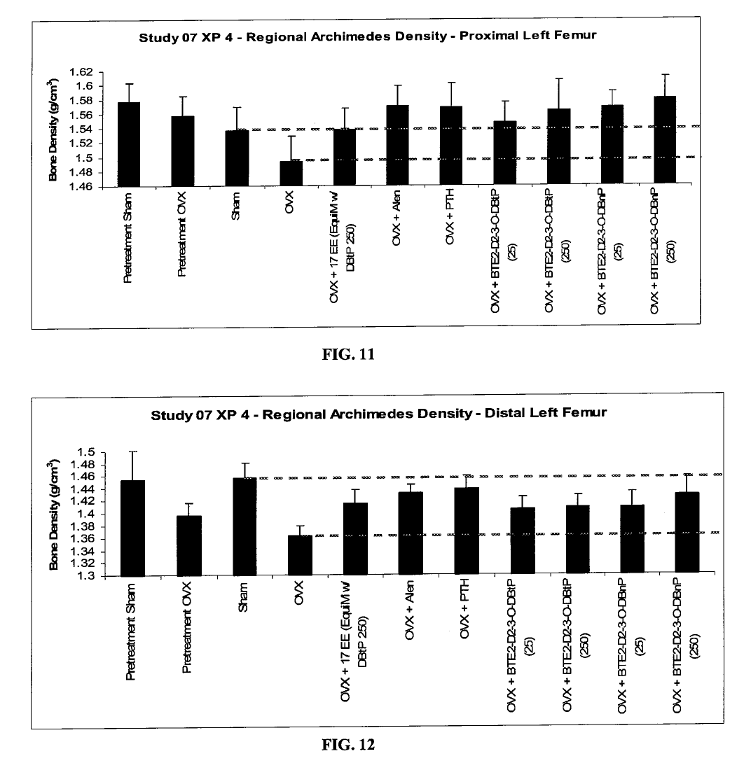

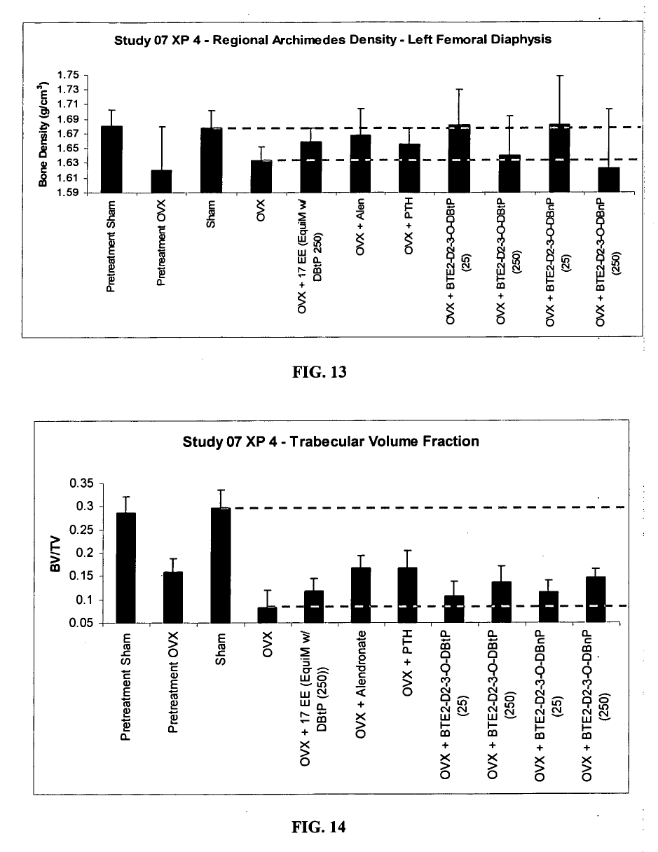

Regional bone density was also assessed, as described above, with data presented in FIGS. 11, 12, and 13. With reference to FIGS. 11, 12, and 13, treatment with the compounds of Formulas 66 (BTE2-D2-3-O-di-n-butyl ester) and 67 (BTE2-D2-3-O-dibenzyl ester) affected regional bone density. Animals receiving the compounds of Formulas 66 and 67 were shown to have increased regional bone densities.

Volume Fraction.

Bone density was further assessed by measuring trabecular volume fraction using ex vivo micro-computed tomography. FIG. 14 includes the trabecular volume fraction data for control animals and animals receiving various doses of the compounds of Formulas 66 (BTE2-D2-3-O-di-n-butyl ester) and 67 (BTE2-D2-3-O-dibenzyl ester). These data indicate that bone density is increased, as compared to OVX animals, in animals receiving the compounds of Formula 66 and 67.

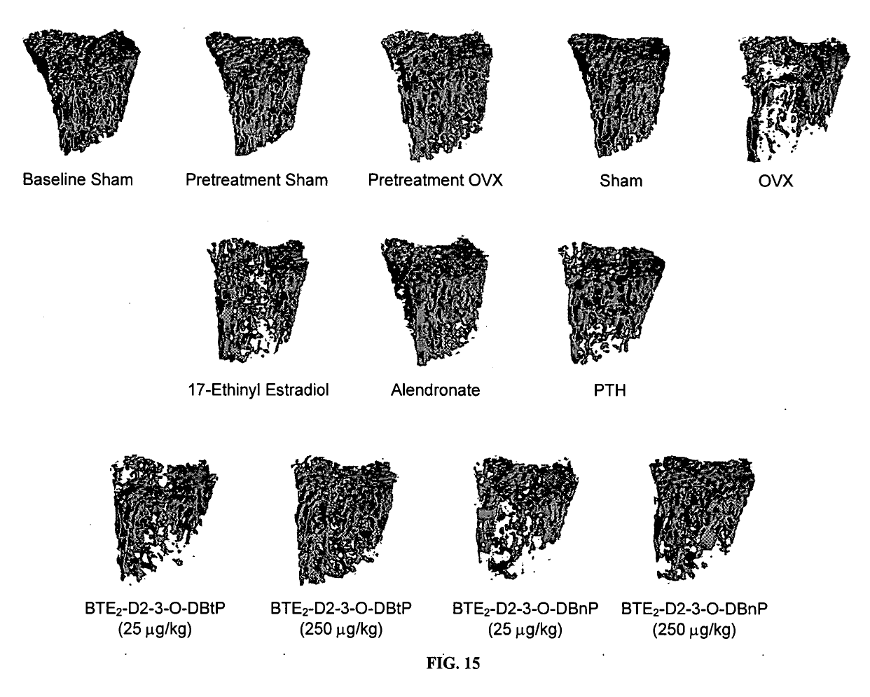

With reference to FIG. 15, three-dimensional images were reconstructed using high resolution image data collected using the customized micro-CT system described above. These data indicate that the compounds of Formulas 66 (BTE2-D2-3-O-di-n-butyl ester) and 67 (BTE2-D2-3-O-dibenzyl ester) affected an increase in bone density.

Bone Strength.

Mechanical Competence.

Samples are collected and an indentation test is performed as described above.

The compounds of Formulas 66 (BTE2-D2-3-O-di-n-butyl ester) and 67 (BTE2-D2-3-O-dibenzyl ester) are found to increase bone strength.

Bone Formation and Turnover.

Collagen Type I.

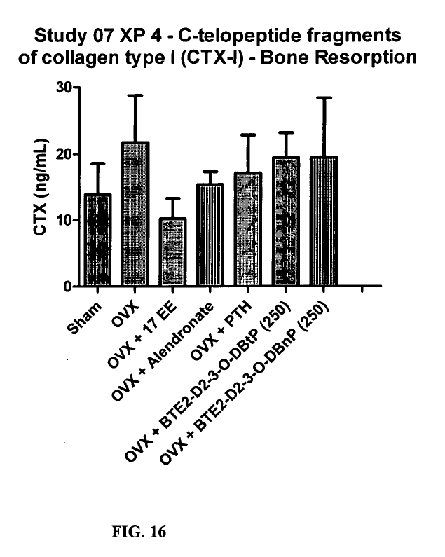

Bone resorption or osteoclast-mediated breakdown of collagen type I in bone was assessed by measuring the C-telopeptide fragment of collagen type I (CTX-I), as described above. With reference to FIG. 16, it is shown that treatment with 250 μg/kg doses of the compounds of Formulas 66 (BTE2-D2-3-O-di-n-butyl ester) and 67 (BTE2-D2-3-O-dibenzyl ester) do not inhibit production of CTX-I, indicating that bone resorption is not inhibited and that the compound acts in a similar manner to known anabolic agent, parathyroid hormone 1-34 (PTH). In contrast, 17-ethinyl estradiol (7-LEE) decreased the production of CTX-1 to below sham levels.

Osteocalcin.

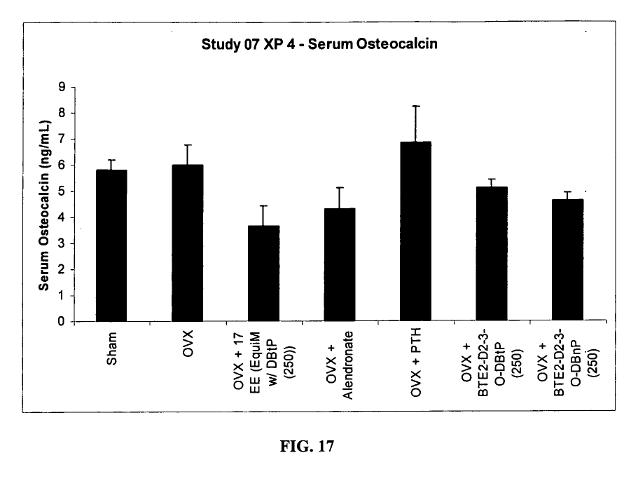

With reference to FIG. 17, serum osteocalcin levels were measured as described above. In the animals treated with the compounds of Formulas 66 (BTE2-D2-3-O-di-n-butyl ester) and 67 (BTE2-D2-3-O-dibenzyl ester) serum osteocalcin levels were lower than in OVX controls.

In vivo Bone Anabolic Measurements.

Animals are administered control agents and the compound, samples are collected, and data are obtained, as described above. The compounds of Formulas 66 (BTE2-D2-3-O-di-n-butyl ester) and 67 (BTE2-D2-3-O-dibenzyl ester) are found to increase new bone area.

Additional Compounds including Bone Active Portion (R_{A}) derived from Estradiol.

Animals are administered control agents and the compounds of Formulas 65, 68-71, and 133-139. Compounds wherein the bone active portion is derived from estradiol are selected as examples of compounds including a bone active portion derived from a steroidal estrogenic agent. Samples are collected and studied as described above.

Body Weight.

Body weight is measured. The compounds of Formulas 65, 68-71, and 133-139 have limited or no effect on body weight.

Uterine Mass.

Uterine mass is obtained. The compounds of Formulas 65, 68-71, and 133-139 have a limited or no effect on uterine mass.

Lipid Metabolism.

Total cholesterol, high-density lipoprotein (HDL), and low-density lipoprotein (LDL) in collected serum samples are quantitatively determined. The compounds of Formulas 65, 68-71, and 133-139 have limited or no effect on lipid metabolism.

Bone Density Effects.

Whole Bone Density and Regional Femoral Density.

Samples are collected as described above. Whole bone density and regional bone density are assessed. The compounds of Formulas 65, 68-71, and 133-139 increase bone density.

Volume Fraction—Trabecular, Cortical and Whole Bone.

Samples are collected as described above. Data revealing the volume fraction (BV/TV) occupied by trabecular (cancellous) bone tissue, cortical bone tissue, and whole bone tissue is obtained. The compounds of Formulas 65, 68-71, and 133-139 increase bone density.

Bone Strength.

Mechanical Competence.

Samples are collected and an indentation test is performed as described above.

The compounds of Formulas 65, 68-71, and 133-139 increase bone strength.

Bone Formation and Turnover.

In vivo Bone Anabolic Measurements.

Animals are administered control agents and the compound, samples are collected, and data are obtained, as described above. The compounds of Formulas 65, 68-71, and 133-139 increase new bone area.

Compound including Bone Active Portion (R_{A}) derived from other Estrogenic Agents.

Animals are administered control agents and the compound of the presently-disclosed subject matter. Compounds wherein the bone active portion is derived from genistein are selected as examples of compounds including a bone active portion derived from a non-steroidal estrogenic agent. Compounds wherein the bone active portion is derived from genistein can include the compounds of the exemplary synthesis set forth in these examples, wherein the Blocking Group can be derived from: phosphoric acid, di-n-butyl phosphate, dibenzyl phosphate, diisopropyl phosphate, di-tert-butyl phosphate, di-2-ethylhexyl phosphate, or didodecyl phosphate. Compounds wherein the bone active portion is derived from another estrogenic agent in accordance with the presently-disclosed subject matter are also selected. Samples are collected and studied as described above.

Body Weight.

Body weight is measured. The compounds of the presently-disclosed subject matter have limited or no effect on body weight.

Uterine Mass.

Uterine mass is obtained. The compounds of the presently-disclosed subject matter have limited or no effect on uterine mass.

Lipid Metabolism.

Total cholesterol, high-density lipoprotein (HDL), and low-density lipoprotein (LDL) in collected serum samples are quantitatively determined. The compounds of the presently-disclosed subject matter have limited or no effect on lipid metabolism.

Bone Density Effects.

Whole Bone Density and Regional Femoral Density.

Samples are collected as described above. Whole bone density and regional bone density are assessed. The compounds of the presently-disclosed subject matter increase bone density.

Volume Fraction—Trabecular, Cortical and Whole Bone.

Samples are collected as described above. Data revealing the volume fraction (BV/TV) occupied by trabecular (cancellous) bone tissue, cortical bone tissue, and whole bone tissue is obtained. The compounds of the presently-disclosed subject matter increase bone density.

Bone Strength.

Mechanical Competence.

Samples are collected and an indentation test is performed as described above.

The compounds of the presently-disclosed subject matter increase bone strength.

Bone Formation and Turnover.

In vivo Bone Anabolic Measurements.

Animals are administered control agents and the compound, samples are collected, and data are obtained, as described above. The compounds of the presently-disclosed subject matter increase new bone area.

### REFERENCES

Throughout this document, various references are mentioned. All such references are incorporated herein by reference, including the references set forth in the following list:
Delmas P D, et al. The Use of Biochemical Markers of Bone Turnover in Osteoporosis. Osteoporosis Int. suppl. 6 S2-17 (2000).
Garrett I R, Chen D, Gutierrez G, Rossini G, Zhao M, Escobedo A, Kim K B, Hu S, Crews C M, and Mundy G R (2003) Selective inhibitors of the osteoblast proteasome stimulate bone formation in vivo and in vitro. J Clin Invest 111: 1771-1782.
Keenan M J, Hegsted M, Jones K L, Delany J P, Kime J C, Melancon L E, Tulley R T, Hong K D. Comparison of bone density measurement techniques: DXA and Archimedes' principle. J Bone Miner Res 1997; 12:1903-7.
Mundy G R, Garrett R, Harris S E, Chan J, Chen D, Rossini G, Boyce B, Zhao M, and Gutierrez G (1999) Stimulation of bone formation in vitro and in rodents by statins. Science 286:1946-1949.
Riggs B L, and Parfitt A M, “Drugs Used to Treat Osteoporosis: The Critical Need for a Uniform Nomenclature Based on Their Action on Bone Remodeling,” J. Bone and Mineral Res. 20:2 (2005).
Seibel M J. Biochemical Markers of Bone Turnover. Clin Biochem Rev. 2005; 26:97-122. U.S. Pat. No. 7,196,220 to Pierce, et al., entitled “Bone Targeting Compounds for Delivering Agents to the Bone for Interaction Therewith.”
U.S. patent application Ser. No. 12/036,057 to Pierce, et al., entitled, “Methods and Compounds for the Targeted Delivery of Agents to Bone for Interaction Therewith.”
U.S. patent application Ser. No. 11/674,753 to Pierce, et al., entitled, “Bone Targeting Compounds for Delivering Agents to the Bone for Interaction Therewith.”
U.S. patent application Ser. No. 11/022,024 to Pierce, et al., entitled, “Compounds for diagnosis, treatment, and prevention of bone injury and metabolic disorders.”
PCT Patent Publication No. WO/0066613 of Pierce, et al., entitled, “Bone Targeting Agents for Osteoporosis.”

What is claimed is:

1. A compound of the formula: 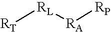
or pharmaceutically acceptable salts, solvate, ethers, or ester thereof wherein R_{T} is a Bone Targeting Portion, according the formula: 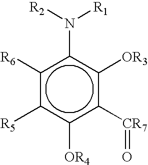
wherein R_{T} is connected at R₁, R₂, R₄, or R₇, to R_{L};
wherein
R₁ is hydrogen, lower alkyl, alkyl, aryl lower alkyl, or aryl, when R_{T} is not connected at R₁ to R_{L};
R₂ is hydrogen, lower alkyl, alkyl, aryl lower alkyl, or aryl, when R_{T} is not connected at R₂ to R_{L};
R₃ is hydrogen, lower alkyl, alkyl, aryl lower alkyl, aryl, or carbonyl-containing;
R₄ is hydrogen, lower alkyl, alkyl, aryl lower alkyl, aryl, or carbonyl-containing, when R_{T} is not connected at R₄ to R_{L};
R₅ and R₆ are independently hydrogen, lower alkyl, or alkyl, or R₅ and R₆, taken together with the carbon atoms to which they are bonded, form a ring containing about 6 to about 14 carbon atoms and up to a total of about 18 carbon atoms, which formed ring can be monocyclic, bicyclic, or tricyclic, wherein the ring can have substituents, including heteroatoms;
R₇ is hydroxy, lower alkoxy, or NR₈, R₉, when R_{T} is not connected at R₇ to R_{L};
R₈ and R₉ are independently hydrogen, or lower alkyl;
wherein R_{A} is a Bone Active Portion derived from an estrogenic agent;
wherein R_{L} is a Linking Portion that separates and connects the Bone Targeting Portion and the Bone Active Portion; and
wherein R_{P} is a blocking group that reduces or eliminates the estrogenic activity of the Bone Active Portion.

2. The compound of claim 1, wherein R₃ is hydrogen.

3. The compound of claim 1, wherein R₅ and R₆ are hydrogen.

4. The compound of claim 1, wherein R₇ is NR₈R₉.

5. The compound of claim 3, wherein R₈ and R₉ are both hydrogen.

6. The compound of claim 1, wherein
R₁ is hydrogen or aryl, when R_{T} is not connected at R₁ to R_{L};
R₂ is hydrogen or aryl, when R_{T} is not connected at R₂ to R_{L};
R₄ is lower alkyl, or hydrogen, when R_{T} is not connected at R₄ to R_{L};
R₃, R₅, and R₆ are each hydrogen; and
R₇ is NH₂, when R_{T} is not connected at R₇.

7. The compound of claim 1, according to the formula:

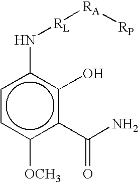

8. The compound of claim 1, according to the formula:

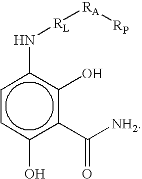

9. The compound of claim 1, according to the formula:

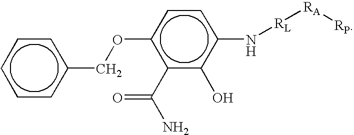

10. The compound of claim 1, according to the formula:

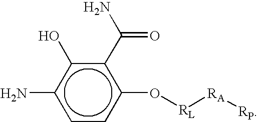

11. The compound of claim 1, wherein R₅ and R₆ taken together with the carbon atoms to which they are attached form a ring containing between 6 and 14 ring carbon atoms, the ring being monocyclic, bicyclic, or tricyclic.

12. The compound of claim 1, wherein
R₁ is hydrogen, lower alkyl, or aryl, when R_{T} is not connected at R₁ to R_{L};
R₂ is hydrogen or aryl, when R_{T} is not connected at R₂ to R_{L};
R₃ is hydrogen, or lower alkyl;
R₄ is hydrogen, when R_{T} is not connected at R₄;
R₅, and R₆ are each hydrogen; and
R₇ is NH₂, when R_{T} is not connected at R₇.

13. The compound of claim 12, wherein R₁ is hydrogen, and R₃ is hydrogen.

14. The compound of claim 1, wherein the Blocking Group is derived from: esters and ethers formed by condensation of lower alkyl, alkyl, or aryl; and sulfates, phosphates, phosphonates, bisphosphonates, substituted bisphosphonates, and salts, esters, or ethers thereof.

15. The compound of claim 14, wherein the Blocking Group is derived from phosphates, phosphonates, bisphosphonates, substituted bisphosphonates, and salts, esters, or ethers thereof

16. The compound of claim 1, wherein the blocking group is derived from phosphate, etidronate, clodronate, tiludronate, pamidronate, alendronate, neridronate, olpadronate, ibandronate, risedronate, zoledronate, minodronate, incadronate, or EB-1053.

17. The compound of claim 1, wherein the Bone Active Portion is derived from an estrogenic agent selected from: estradiol; estrone; estriol; an estrogen precursor;
an estrogen analogue; an estrogen metabolite; tibolone; 2-methoxyestradiol; genistein;
resveratrol; daidzein; glycitein; formononetin; biochanin A; diethylstilbestrol; enterodiol;
enterolactone; hexestrol; xenoestrogens; phytoestrogens; mycoestrogens; coumestrol; a coumestan; isoflavonoids; ipriflavone; secoisolariciresinol diglycoside; and lignan phytoestrogens.

18. The compound of claim 1, according to the formula:

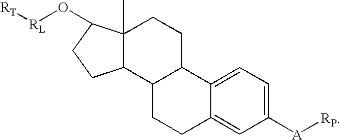

19. The compound of claim 18, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

20. The compound of claim 1, according to the formula:

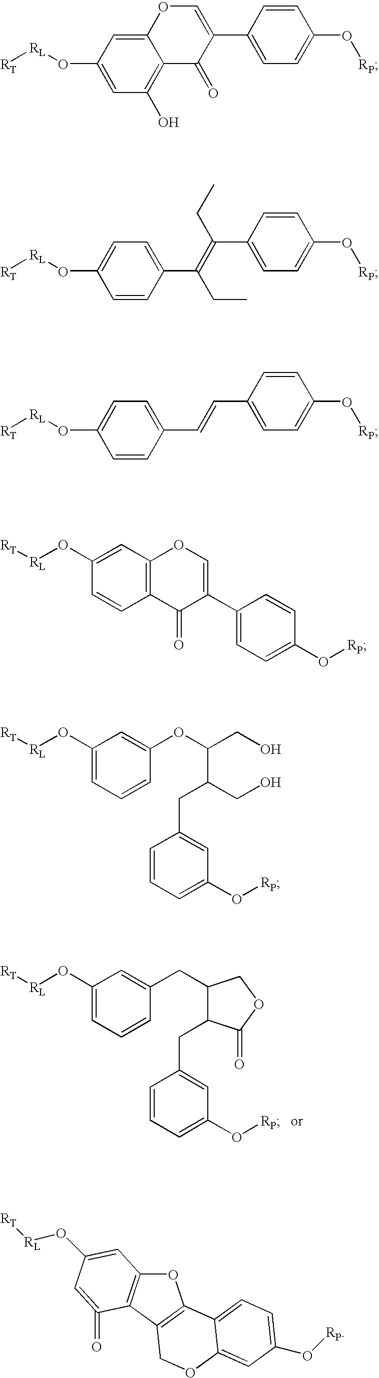

21. The compound of claim 20, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

22. The compound of claim 1, according to the formula: 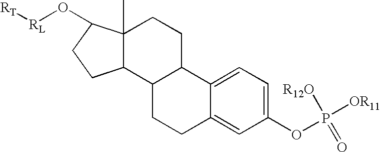
wherein R₁₁ and R₁₂ are independently hydrogen, lower alkyl, alkyl, or aryl.

23. The compound of claim 22, wherein R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

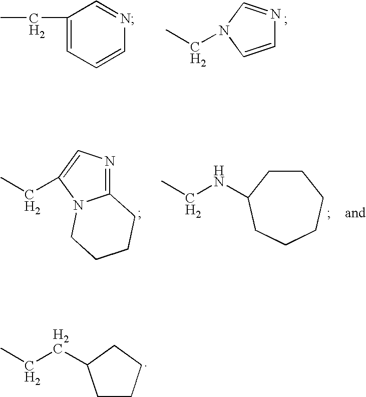

24. The compound of claim 1, according to the formula: 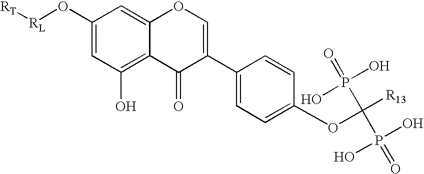
wherein R₁₃ is hydrogen, lower alkyl, alkyl, or aryl.

25. The compound of claim 24, wherein R₁₃ is selected from: —H; —CH₃; —Cl; 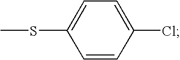
—(CH₂)₂NH₂; —(CH₂)₃NH₂; —(CH₂)₄NH₂; —(CH₂)₅NH₂; —(CH₂)₂N(CH₃)₂;

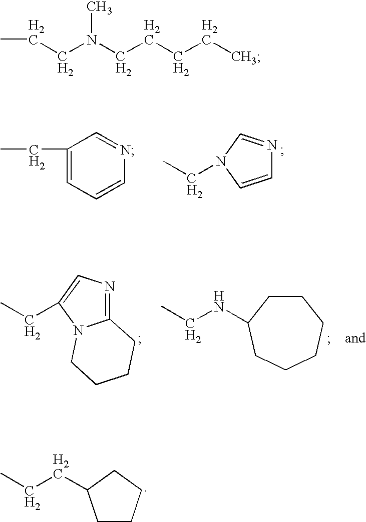

26. The compound of claim 1, according to the formula: 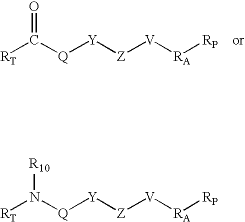
wherein
R₁₀ is independently hydrogen or lower alkyl;
Q is a straight or branched alkylene group, containing 1 to about 10 carbon atoms on a main chain, and up to a total of about 20 carbon atoms; 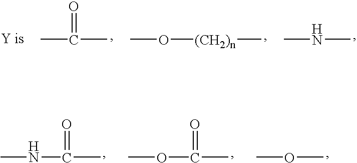
or a chemical bond;
Z is 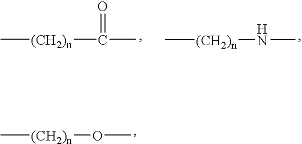
or a chemical bond;
V is 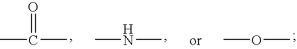
provided Y-E-V is 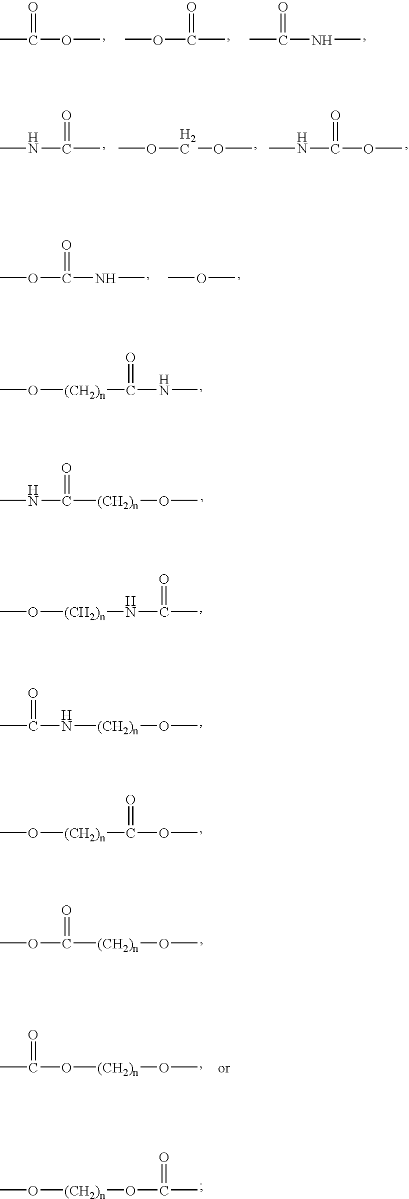
and n is an integer from 0 to 6.

27. The compound of claim 26, according to the formula:

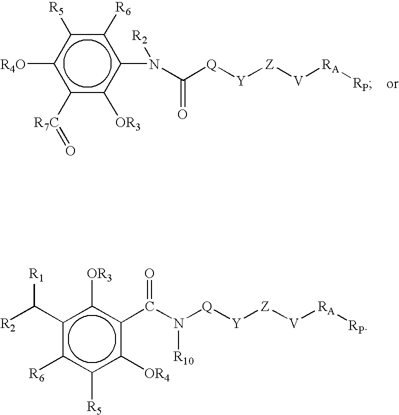

28. The compound of claim 27, according to the formula: 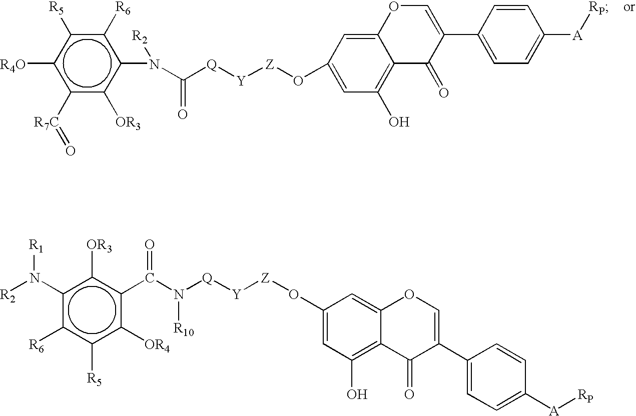
wherein A is a heteroatom.

29. The compound of claim 27, according to the formula: 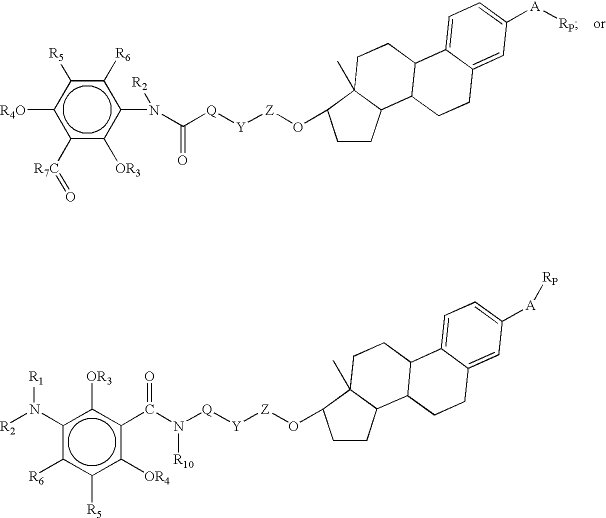
wherein A is a heteroatom.

30. The compound of claim 29, according to the formula:

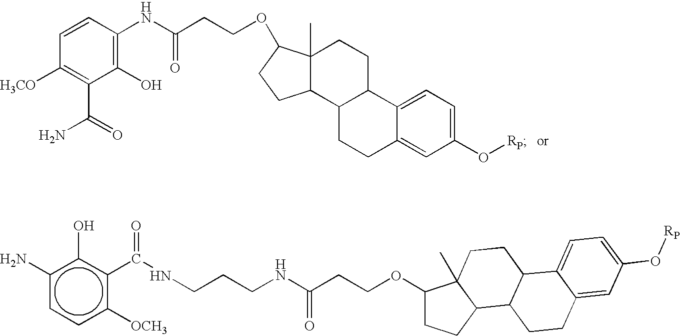

31. The compound of claim 30, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

32. The compound of claim 30, according to the formula:

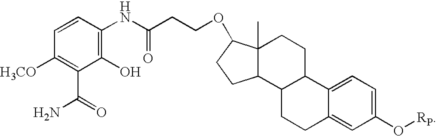

33. The compound of claim 32, according to the formula: 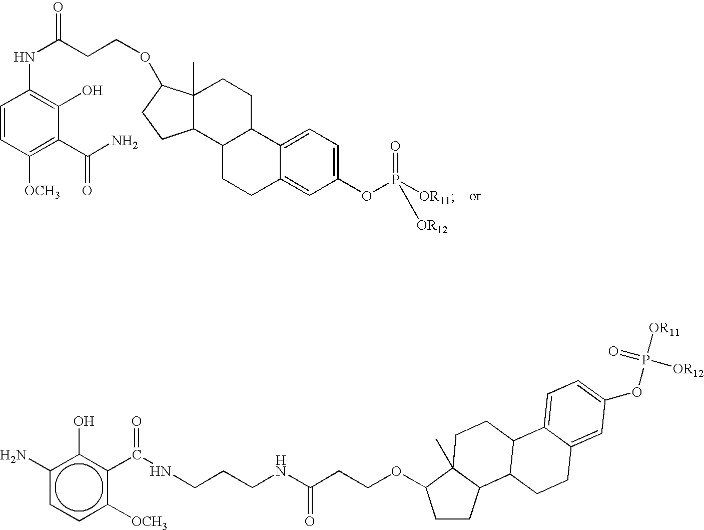
wherein R₁₁ and R₁₂ are independently hydrogen, lower alkyl, alkyl, or aryl.

34. The compound of claim 33, wherein R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

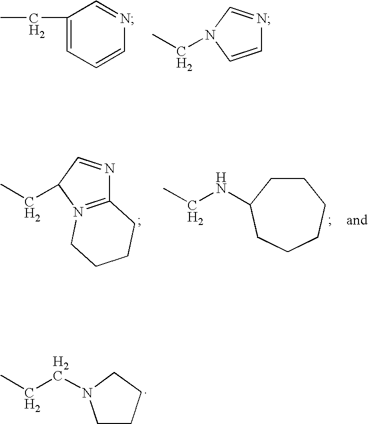

35. The compound of claim 33, according to the formula:

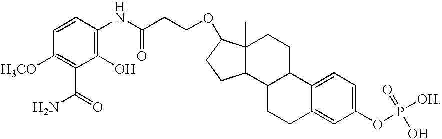

36. The compound of claim 33, according to the formula:

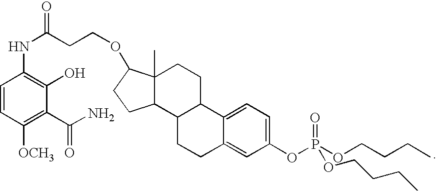

37. The compound of claim 33, according to the formula:

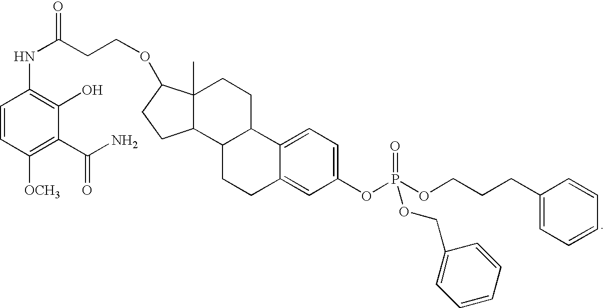

38. The compound of claim 33, according to the formula:

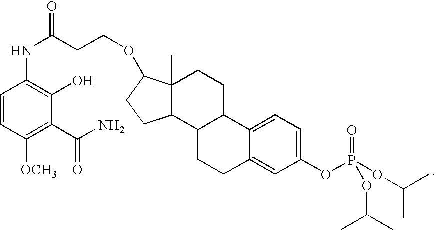

39. The compound of claim 33, according to the formula:

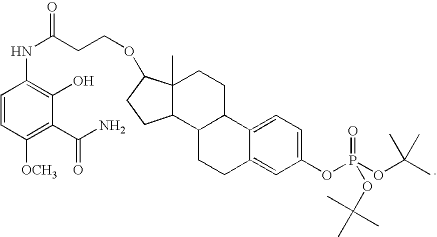

40. The compound of claim 33, according to the formula:

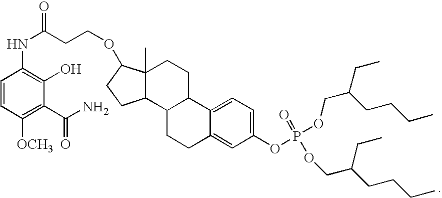

41. The compound of claim 33, according to the formula:

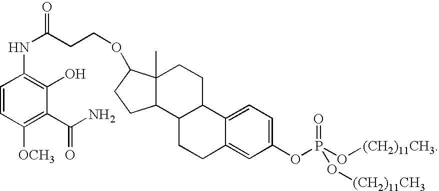

42. The compound of claim 1, according to the formula: 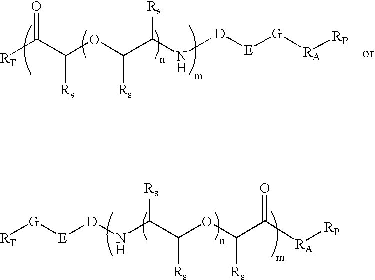
wherein m is 1-3, n is 1-4, and when m>1, each n is independently 1-4;
wherein each R_{S} is independently hydrogen, lower alkyl, or lower alkyl with heteroatoms;
wherein D and G are independently covalent bond, carbonyl, epoxy, or anhydride; and
wherein E is
covalent bond,
(CT2)ᵣ, where T is hydrogen, hydroxy, or lower alkyl, and where r is 0-8, or
(C)ᵣ, where r is 2-8, and where the carbons are unsaturated or partially saturated with hydrogen.

43. The compound of claim 42, according to the formula:

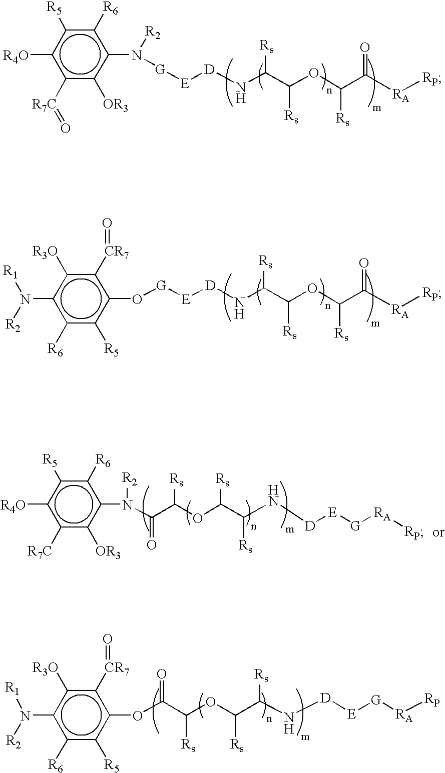

44. The compound of claim 43, according to the formula:

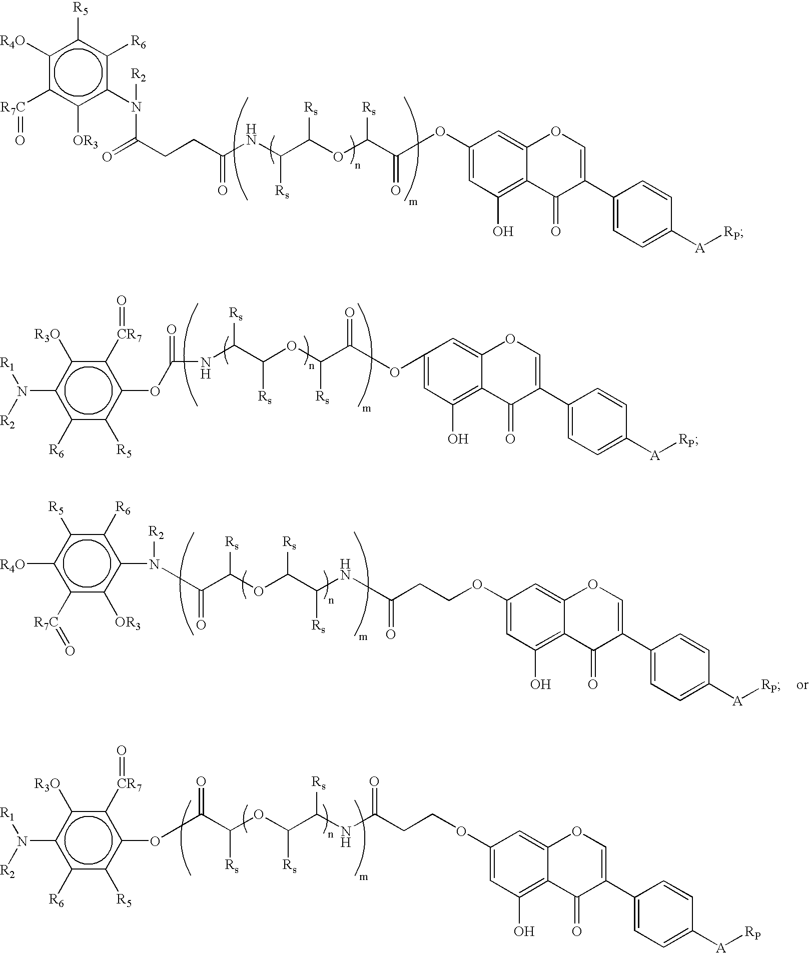

45. The compound of claim 43, according to the formula:

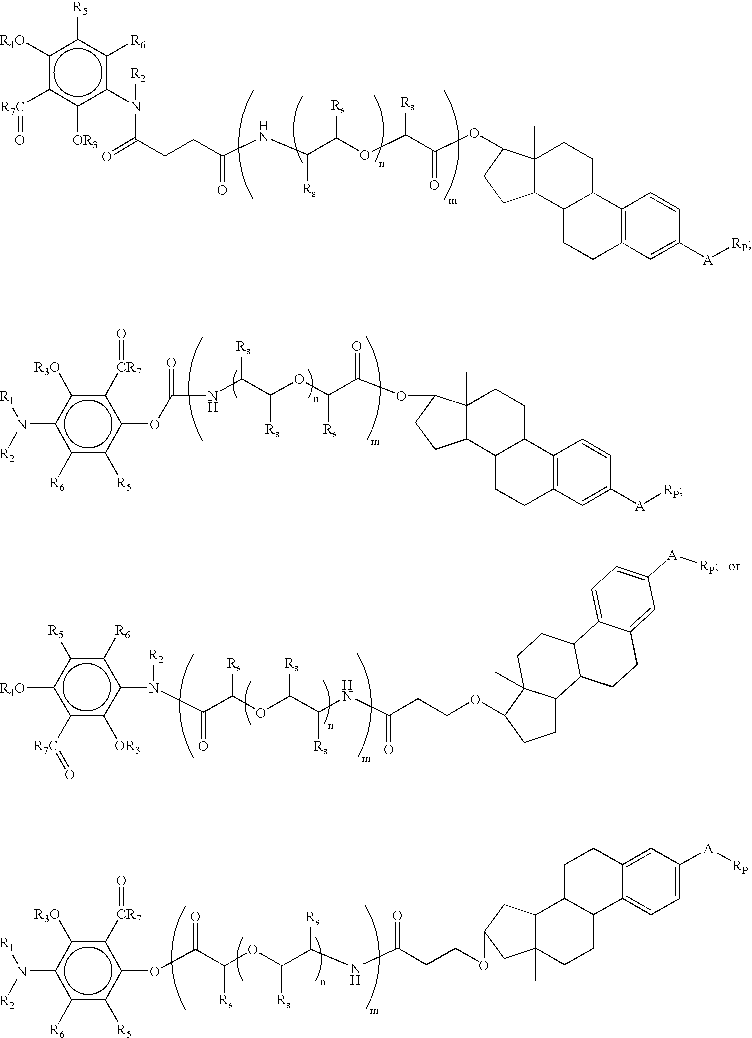

46. The compound of claim 45, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

47. The compound of claim 45, according to the formula:

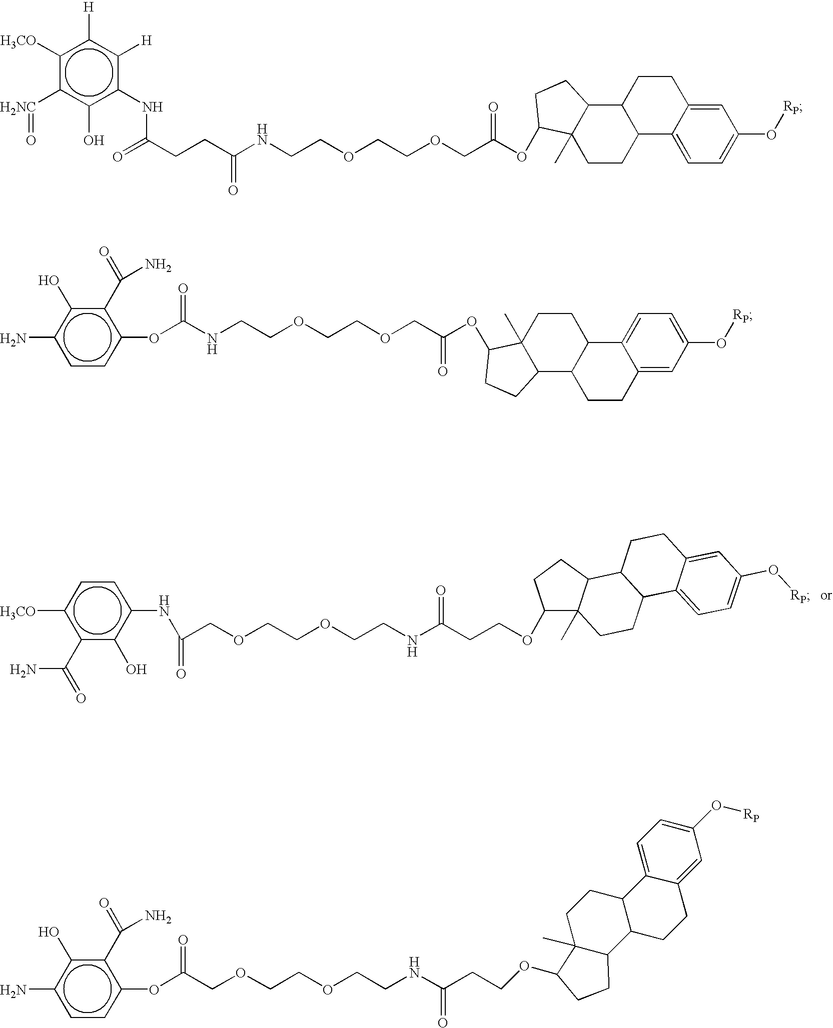

48. The compound of claim 47, according to the formula:

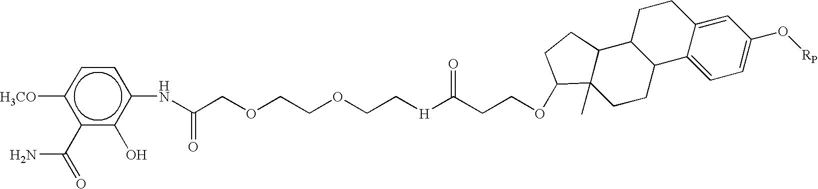

49. The compound of claim 48, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

50. The compound of claim 45, according to the formula: 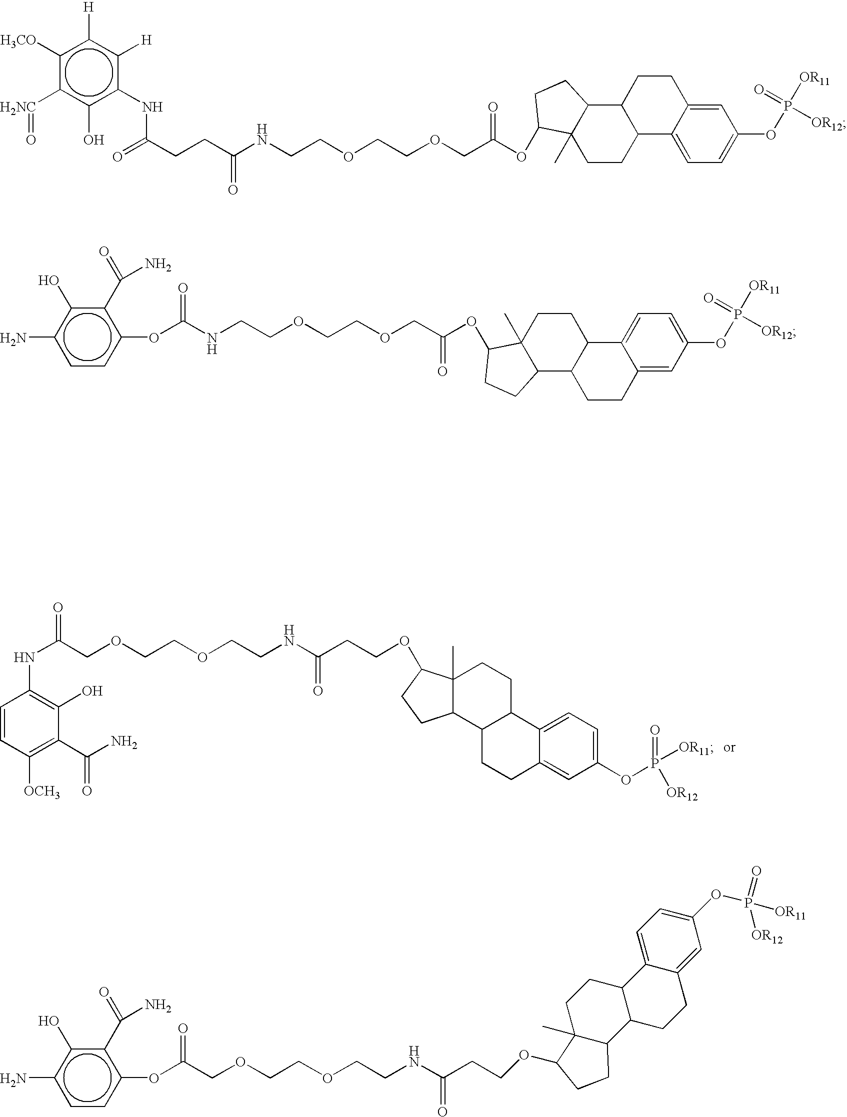
wherein R₁₁ and R₁₂ are independently hydrogen, lower alkyl, alkyl, or aryl.

51. The compound of claim 50, wherein R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

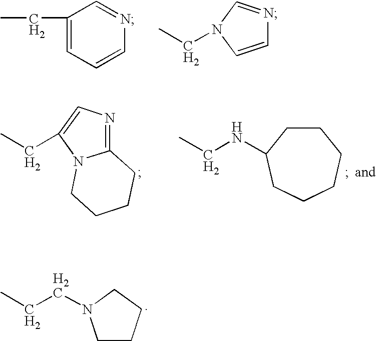

52. The compound of claim 50, according to the formula:

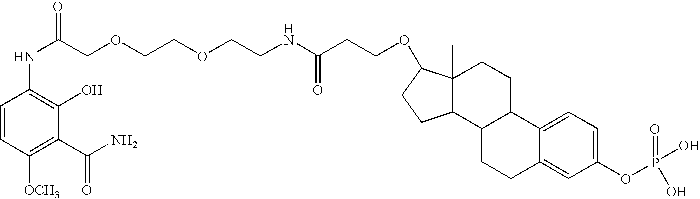

53. The compound of claim 50, according to the formula:

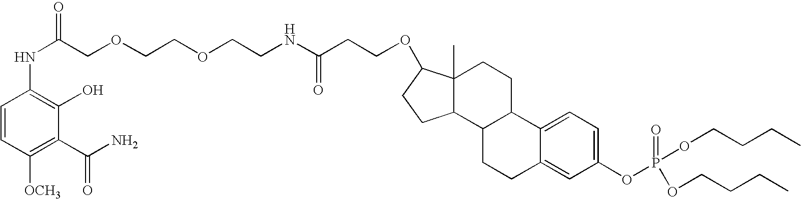

54. The compound of claim 50, according to the formula:

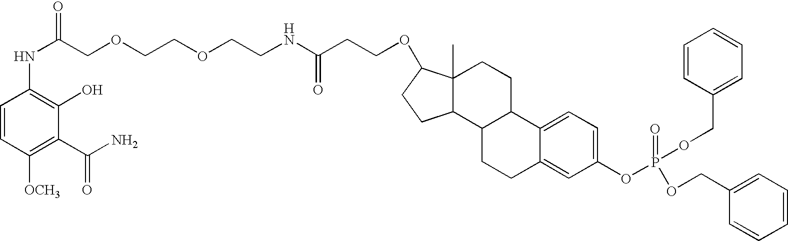

55. The compound of claim 50, according to the formula:

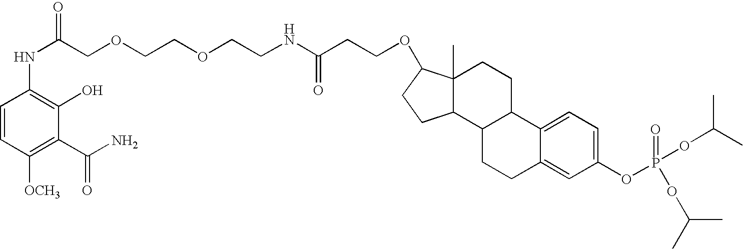

56. The compound of claim 50, according to the formula:

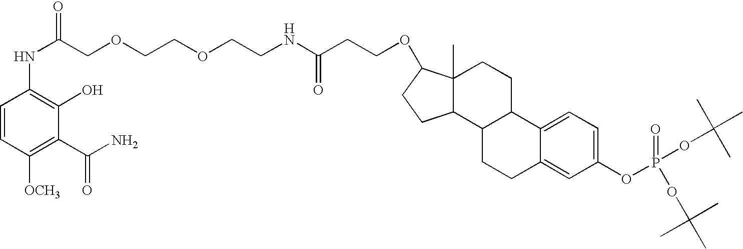

57. The compound of claim 50, according to the formula:

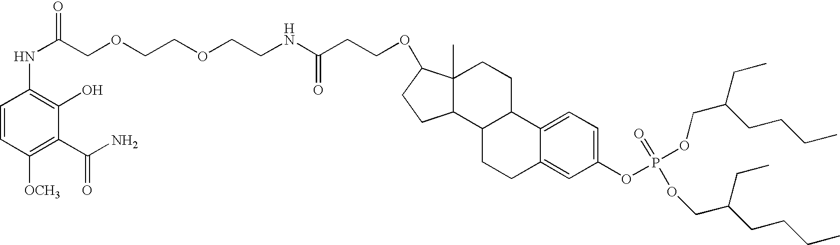

58. The compound of claim 50, according to the formula:

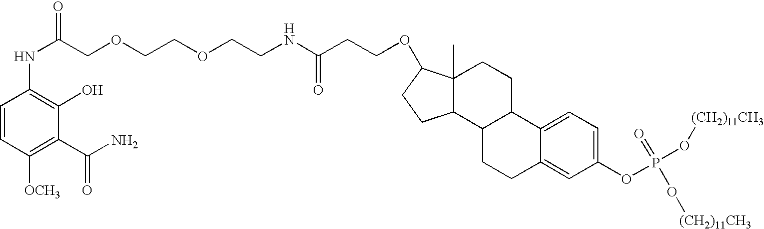

59. The compound of claim 1, according to the formula: 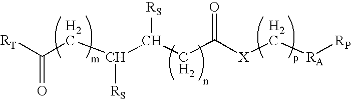
wherein m is 0-3, n is 0-3, and p is 0-4;
wherein each R_{S} is independently hydrogen or hydroxy; and
wherein X is O, NH, S, or covalent bond.

60. The compound of claim 59, according to the formula:

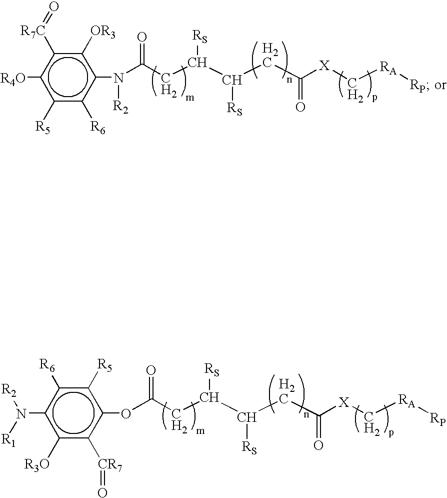

61. The compound of claim 60, according to the formula:

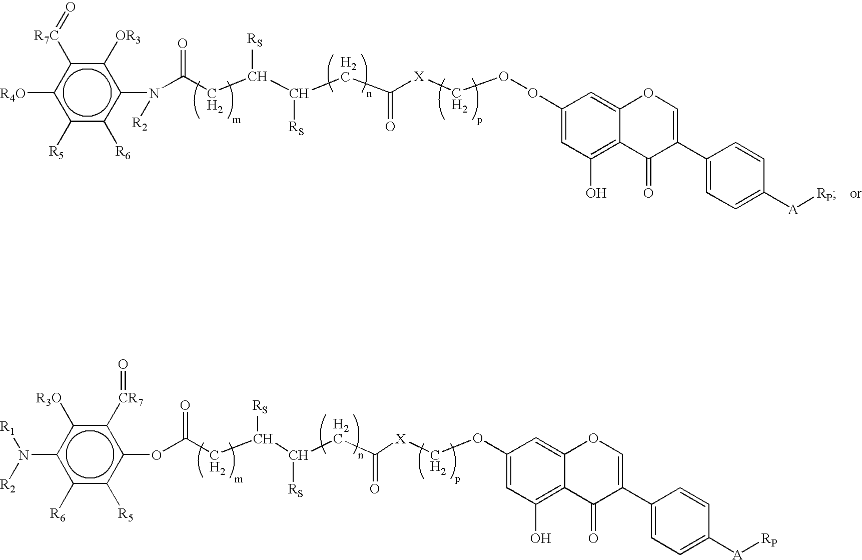

62. The compound of claim 60, according to the formula:

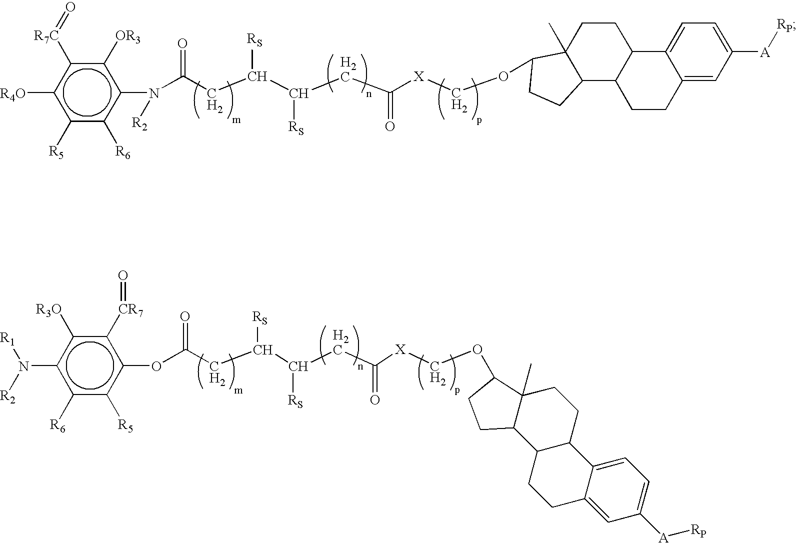

63. The compound of claim 62, wherein R_{P} is selected from phosphoric acid, di-n-butyl phosphate, dibenzyl phosphate, diisopropyl phosphate, di-tert-butyl phosphate, di-2-ethylhexyl phosphate, or didodecyl phosphate.

64. The compound of claim 62, according to the formula:

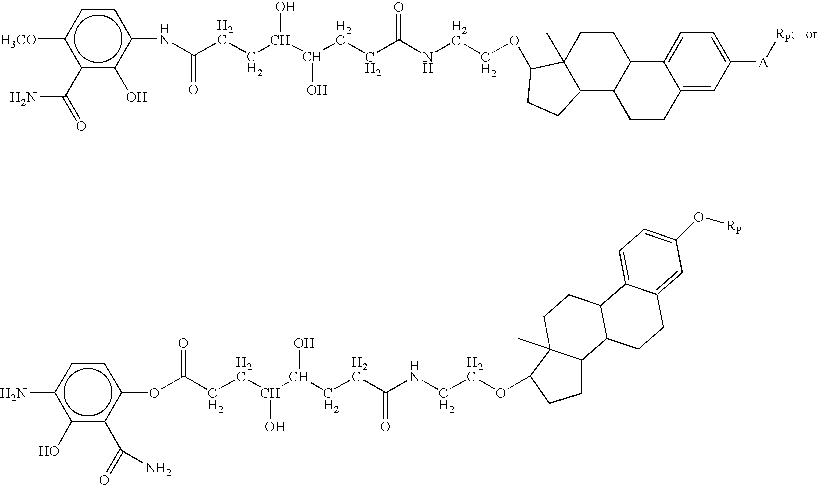

65. The compound of claim 64, according to the formula: 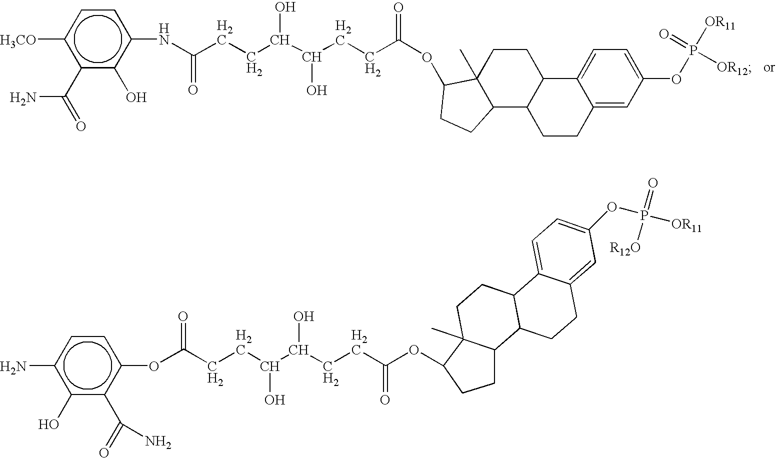
wherein R₁₁ and R₁₂ are independently hydrogen, lower alkyl, alkyl, or aryl.

66. The compound of claim 65, wherein R₁₁ and R₁₂ are independently selected from: hydrogen; methyl; n-butyl; benzyl; isopropyl; tert-butyl; 2-ethylhexyl; dodecyl; N-methyl-N-propylpentan-1-amine; —(CH₂)₂NH₂; —(CH₂)₃NH₂; —(CH₂)₄NH₂; —(CH₂)₅NH₂; —(CH₂)₂N(CH₃)₂;

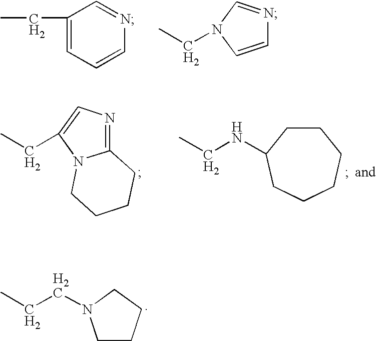

67. A method for treating a bone condition in an subject, comprising:
administering to the subject an effective amount of the compound of claim 1.

68. The method of claim 67, wherein the bone condition is a metabolic bone disorder.

69. The method of claim 70, wherein the metabolic bone disorder is osteoporosis.

70. The method of claim 67, wherein the bone condition is a fracture.

71. The method of claim 67, wherein administering the compound has an anti-catabolic effect and/or an anabolic effect on the bone of the subject.

72. The method of claim 71, wherein administering the compound has an anabolic effect on the bone of the subject.
